(19) United States
(12) Patent Application Publication
(10) Pub. No.: US 2014/0050778 A1
(43) Pub. Date: Feb. 20, 2014

(54) NUCLEIC ACID BINDING COMPOUNDS, METHODS OF MAKING, AND USE THEREOF

(71) Applicants: Benjamin L. Miller, Penfield, NY (US); Leslie O. Ofori, Rochester, NY (US); Anna V. Gromova, Rochester, NY (US)

(72) Inventors: Benjamin L. Miller, Penfield, NY (US); Leslie O. Ofori, Rochester, NY (US); Anna V. Gromova, Rochester, NY (US)

(73) Assignee: UNIVERSITY OF ROCHESTER, Rochester, NY (US)

(21) Appl. No.: 13/976,852

(22) PCT Filed: Dec. 28, 2011
(86) PCT No.: PCT/US11/67576

§ 371 Date: Oct. 24, 2013

Related U.S. Application Data
(60) Provisional application No. 61/427,752, filed on Dec. 28, 2010.

(51) Int. Cl. C07K 5/087; C07K 5/09

(52) U.S. Cl. CPC C07K 5/0812; C07K 5/0815
USPC 424/450; 530/300; 530/323; 435/235.1; 514/3.8

(57) ABSTRACT

The present invention relates to oligomer compounds, including dimers and trimers, formed by a disulfide, sulfinyl thio, olefin or hydrocarbon bond, or a hydrazone exchange bond between two or more monomers. Methods of making the monomers and the oligomers is also disclosed. Use of the compounds for inhibiting the activity of target RNA molecules, particularly those having a secondary structure that include a stem or stem-loop formation. Dimer compounds capable of inhibiting the activity of an HIV-1 RNA frameshifting stem-loop and a (CUG)n expanded repeat stem-loop are disclosed, as are methods of treating diseases associated with these target RNA molecules.

This application claims the benefit of U.S. Provisional Patent Application Ser. No. 61/427,752, filed Dec. 28, 2010, which is hereby incorporated by reference in its entirety.

This invention was made with government support under grant numbers P30A1078498, 5R21NS071023, AR049077, and U54NS48843 awarded by the National Institutes of Health. The government has certain rights in the invention.

### FIELD OF THE INVENTION

The present invention relates to nucleic acid binding compounds, including monomeric compounds and homo- and hetero-dimeric and oligomeric compounds formed by covalent binding of the monomeric compounds. The present invention is also directed to methods of making and using these compounds.

### BACKGROUND OF THE INVENTION

High affinity, sequence-selective recognition of RNA by synthetic molecules is increasingly recognized as a key strategic goal for the production of novel therapeutics and biochemical probes (Thomas et al., “Targeting RNA with Small Molecules,” Chem. Rev. 108:1171-1224 (2008)). The importance of this area is accentuated by the ever-increasing pace of discovery of new RNA sequences with biochemically important (and, therefore, potentially biomedically important) functions. Among the many recent advances in this area is the recognition that many noncoding RNAs (“ncRNA”) present in the eukaryotic RNAome play a direct role in controlling cellular processes and disease (Wahlestedt, “Natural Antisense and Noncoding RNA Transcripts as Potential Drug Targets,” Drug Discovery Today 11:503-508 (2006)). Human diseases believed to have an ncRNA origin include spinocerebellar ataxia, fragile X-syndrome, diabetes mellitus, myoclonus epilepsy, and the myotonic dystrophies (“DM”) (Gatchel et al., “Diseases of Unstable Repeat Expansion: Mechanisms and Common Principles,” Nat. Rev. Genet. 6:743-755 (2005)). However, to date only a relatively small number of compounds have been reported that bind specific RNA sequences and elicit a desired target RNA-dependent biological response. For these reasons, expanding the pool of sequence-selective RNA-targeted synthetic molecules presents a critically important but under-examined challenge in chemical biology.

The myotonic dystrophies are central examples of a growing family of RNA-mediated diseases (Todd et al., “RNA-mediated Neurodegeneration in Repeat Expansion Disorders,” Ann. Neurol. 67:291-300 (2010); Osborne et al., “RNA-dominant Diseases,” Hum. Mol. Genet. 15:162-169 (2006); Gatchel et al., “Diseases of Unstable Repeat Expansion Mechanisms and Common Principles,” Nat. Rev. Genet. 6:743-755 (2005)). Myotonic dystrophy type 1 (“DM1”) is the most common form of adult-onset muscular dystrophy, affecting about 1 in 8000 people (Brook et al., “Molecular Basis of Myotonic Dystrophy Expansion of a Trinucleotide (CTG) Repeat at the 3′ End of a Transcript Encoding a Protein Kinase Family Member,” Cell 68:799-808 (1992)). An autosomal dominant inherited disease, DM1 results from a CTG repeat expansion (CTG^{exp}) in the 3′-untranslated region of the DM protein kinase gene (DMPK) on chromosome 19q. The expanded CTG is transcribed into long CUG^{exp} repeat mRNA. These RNA repeats sequester RNA-binding proteins such as the MBNL (muscleblind-like) family of splicing regulators, retaining them in the nucleus as foci. This in turn leads to misregulated alternative splicing, or spliceopathy. Myotonic dystrophy type 2 (“DM2”) is caused by an unstable expansion of a CCTG repeat in intron 1 of the zinc finger protein 9 gene (ZNF9) on chromosome 3q. Transcription produces toxic mRNA containing hundreds to thousands of CCUG^{exp}. Like CUG^{exp}, these are also sequestered into foci, and deplete MBNL1 protein from the affected cell (Machuca-Tzili et al., “Clinical and Molecular Aspects of the Myotonic Dystrophies: A Review,” Muscle Nerve 32:1-18 (2005); Warf et al., “Pentamidine Reverses the Splicing Defects Associated with Myotonic Dystrophy,” Proc. Natl. Acad. Sci. U.S.A. 106:18551-18556 (2009)). Currently, there is no pharmaceutical therapy for either DM1 or DM2. However, the molecular understanding of these diseases suggests that displacement of MBNL 1 from its CUG^{exp} or CCUG^{exp} binding sites constitutes an attractive strategy for developing therapies targeting DM (Warf et al., “Pentamidine Reverses the Splicing Defects Associated with Myotonic Dystrophy,” Proc. Natl. Acad. Sci. U.S.A. 106:18551-18556 (2009); Wheeler, “Myotonic Dystrophy: Therapeutic Strategies for the Future,” Neurotherapeutics 5:592-600 (2008)), as was shown using morpholino antisense oligonucleotides (Wheeler et al., “Reversal of RNA Dominance by Displacement of Protein Sequestered on Triplet Repeat RNA,” Science 325:336-339 (2009)). The high cost and challenging pharmacological properties of oligonucleotide-based drugs suggest, however, that alternative approaches to targeting CUG^{exp} RNA are needed.

The first non-nucleic acid-based compounds (compound 1 in FIG. 1 is a representative structure) capable of binding CUG^{exp} RNA and competitively inhibiting CUG^{exp}-MBNL1 binding in vitro were only recently reported (Gareiss et al., “Dynamic Combinatorial Selection of Molecules Capable of Inhibiting the (CUG) Repeat RNA-MBNL1 Interaction In Vitro: Discovery of Lead Compounds Targeting Myotonic Dystrophy (DM1),” J. Am. Chem. Soc. 130:16254-16261 (2008); PCT Publ. No. WO2009015384 to Miller et al.). Several groups have subsequently demonstrated elegant and structurally varied approaches to binding CUG^{exp} and CCUG^{exp} RNA (Warf et al., “Pentamidine Reverses the Splicing Defects Associated with Myotonic Dystrophy,” Proc. Natl. Acad. Sci. U.S.A. 106:18551-18556 (2009); Garcia-Lopez et al., “In Vivo Discovery of a Peptide that Prevents CUG-RNA Hairpin Formation and Reverses RNA Toxicity in Myotonic Dystrophy Models,” Proc. Natl. Acad. Sci. U.S.A. 108:11866-11871 (2011); Arambula et al., “A Simple Ligand that Selectively Targets CUG Trinucleotide Repeats and Inhibits MBNL Protein Binding,” Proc. Natl. Acad. Sci. U.S.A. 106:16068-16073 (2009); Wong et al., “Selective Inhibition of MBNL1-CCUG Interaction by Small Molecules Toward Potential Therapeutic Agents for Myotonic Dystrophy Type 2 (DM2),” Nucl. Acids Res. 39:8881-8890 (2011); Lee et al., “Rational and Modular Design of Potent Ligands Targeting the RNA That Causes Myotonic Dystrophy 2,” ACS Chem. Biol. 4:345-355 (2009); Lee et al., “Controlling the Specificity of Modularly Assembled Small Molecules for RNA via Ligand Module Spacing: Targeting the RNAs That Cause Myotonic Muscular Dystrophy. J. Am. Chem. Soc. 131:17464-17472 (2009)). While this recent upsurge of interest highlights the fact that DM1 and DM2 RNAs are important therapeutic targets, the restoration of MBNL1 activity in vivo by cell-permeable, highly selective CUG^{exp} RNA binders remains an important unmet goal.

More than a quarter century after its identification, the HIV virus continues to be a widespread threat to human health. The success of Highly Active Anti Retroviral Therapy and other interventions in reducing AIDS-related mortality is balanced by the complexity of the therapeutic regimen, and by the known ability of the virus to evolve resistance. Therefore, new therapeutic targets and compounds addressing those targets are of substantial importance. One promising and relatively unexplored therapeutic target is a highly conserved RNA sequence responsible for regulating the production of the Gag-Pol polyprotein via a −1 ribosomal frameshift, which occurs along a “slippery sequence” in gag-pol mRNA. This “frameshift stimulatory sequence” (or “HIV-1 FSS”) includes an upper stemloop and a lower stem, separated by a purine bulge in the most common types of HIV. While compounds that shift the Gag:Gag-Pol ratio by interfering with frameshifting have been shown (see PCT Publ. No. WO2009015384 to Miller et al.), there remains a need to identify cell permeable, selective HIV-1 FSS-targeting compounds having improved affinity.

The present invention is directed to overcoming these and other deficiencies in the art.

### SUMMARY OF THE INVENTION

A first aspect of the present invention relates to a compound having a structure

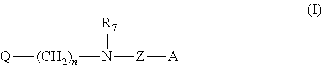

where:

Q is selected from H, NH₂,

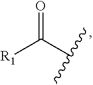

and an inert substrate, where R₁ is selected from a straight or branched chain C₁ to C₆ hydrocarbon, NH₂, and an aromatic or heteroaromatic group;

n is an integer from 0 to about 5;

R₇ is selected from hydrogen and an aromatic or heteroaromatic group;

Z is a peptide containing at least two and up to about ten amino acids, where one of the amino acids is capable of forming a bond by hydrazone exchange, a disulfide bond, sulfinyl thio linkage, olefin bond, or hydrocarbon bond; and

A is selected from a substituted or unsubstituted benzo[g]quinoline, benzo[b][1,8]naphthyridine, and anthyridine connected to Z via a carbonyl linkage or A is selected from

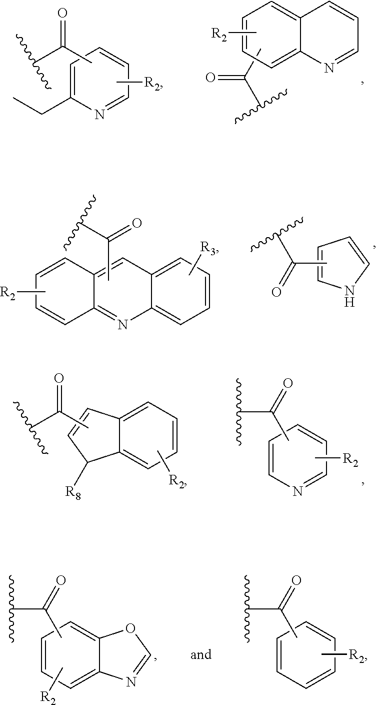

connected to Z via a carbonyl linkage, where R₂ and R₃ are independently selected from the group consisting of hydrogen, methyl, ethyl, propyl, amino, methylamine, ethylamine, dimethylamine, diethylamine, methoxy, ethoxy, propoxy, hydroxyl, cyano, thiocyanato, and aroylhydrazonoalkyl and R₈ is H or C₁-C₃ alkyl.

A second aspect of the present invention relates to a homo- or hetero-dimer compound formed by a disulfide, sulfinyl thio, olefin, or hydrocarbon bond between two monomers having a structure

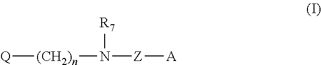

where, for each monomer (I)

Q is independently selected from H, NH₂,

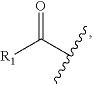

and an inert substrate, where R₁ is selected from a straight or branched chain C₁ to C₆ hydrocarbon and an aromatic or heteroaromatic group;

n is independently an integer from 0 to about 5;

R₇ is selected from hydrogen and an aromatic or heteroaromatic group;

Z is a peptide containing at least two and up to about ten amino acids, where one of the amino acids is capable of forming a disulfide bond, sulfinyl thio linkage, olefin bond, or hydrocarbon bond; and

A is independently hydrogen or an aromatic or heteroaromatic group connected to Z via a carbonyl linkage, provided that in at least one of the monomers A is not hydrogen and is selected from a substituted or unsubstituted benzo[g]quinoline, benzo[b][1,8]naphthyridine, and anthyridine.

A third aspect of the present invention relates to an oligomeric compound comprising two or more monomers selected from the compounds according to the first aspect of the invention.

In one embodiment, the oligomeric compound comprises a first monomer and a second monomer linked by a hydrazone exchange bond, a disulfide bond, sulfinyl thio linkage, olefin bond, or hydrocarbon bond.

In a further embodiment, the oligomeric compound further comprises a third monomer linked to the first monomer or the second monomer by a hydrazone exchange bond, a disulfide bond, sulfinyl thio linkage, olefin bond, or hydrocarbon bond, wherein the bond by which the third monomer is linked to the first or second monomer is different than the bond that links the first and second monomers.

In an alternative embodiment, the oligomeric compound further comprises a third monomer linked to the first monomer or the second monomer by a hydrazone exchange bond, a disulfide bond, sulfinyl thio linkage, olefin bond, or hydrocarbon bond, wherein the bond by which the third monomer is linked to the first or second monomer is the same as the bond that links the first and second monomers.

A fourth aspect of the present invention is directed to a composition comprising a carrier and either (i) a homo- or hetero-dimer compound according to the second aspect of the present invention, or (ii) an oligomeric compound according to the third aspect of the present invention.

A fifth aspect of the present invention relates to a method of inhibiting HIV-1 proliferation. This method involves providing a dimer compound according to the second aspect of the present invention or an oligomeric compound according to the third aspect of the present invention, and contacting an HIV-1 mRNA that encodes Pol polyprotein with the dimer compound or oligomeric compound under conditions effective to alter normal expression of the Pol polyprotein and thereby inhibit HIV-1 proliferation.

A sixth aspect of the present invention relates to a method of treating HIV-1 in a human patient. This method involves administering to a human patient a dimer compound according to the second aspect of the present invention, an oligomeric compound according to the third aspect of the present invention, or a composition according to the fourth aspect of the present invention under conditions effective to alter normal expression of HIV-1 Pol polyprotein, thereby disrupting HIV-1 proliferation to treat the human patient for HIV-1.

A seventh aspect of the present invention is directed to a method of detecting presence of an HIV-1 virus in a sample. This method involves providing a homo- or hetero-dimer compound according to the second aspect of the present invention or an oligomeric compound according to the third aspect of the present invention, where the dimer compound or oligomeric is immobilized on a surface. The immobilized dimer compound or oligomer compound is contacted with a sample under conditions effective to permit an mRNA frameshift regulatory molecule of the HIV-1 virus to bind specifically to the immobilized homo- or hetero-dimer compound or oligomer compound. Presence of the mRNA frameshift regulatory molecule in the sample is detected based on said binding, where detection of the mRNA frameshift regulatory molecule indicates presence of the HIV-1 virus in the sample.

An eighth aspect of the present invention relates to a method of selecting homo- and/or hetero-dimer compounds or oligomer compounds capable of selectively binding an mRNA regulatory sequence comprising a stem or stem/loop formation. This method involves providing a heterogeneous mixture of solution phase monomers each having a structure

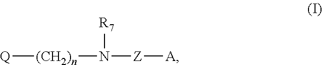

where, for each solution phase monomer,

Q is independently selected from H, NH₂,

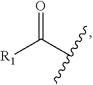

and an inert substrate, where R₁ is selected from a straight or branched chain C₁ to C₆ hydrocarbon and an aromatic or heteroaromatic group;

n is independently an integer from 0 to about 5;

R₇ is selected from hydrogen and an aromatic or heteroaromatic group;

Z is independently a peptide, N-alkylated peptide, or a reduced peptide containing at least two and up to about ten amino acids, where one of the amino acids is capable of forming a disulfide bond, sulfinyl thio linkage, olefin bond, or hydrocarbon bond; and

A is independently hydrogen or an aromatic or heteroaromatic group connected to Z via a carbonyl linkage.

The heterogeneous mixture of solution phase monomers is equilibrated with a labeled mRNA regulatory sequence and a heterogeneous mixture of inert substrate-bound monomers each having a structure

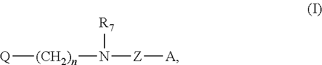

where, for each substrate-bound monomer, Q is an inert substrate and n, R₇, Z, and A are independently selected from the groups defined above, provided that in at least one of the solution phase or substrate-bound monomers A is not hydrogen and is selected from a substituted or unsubstituted benzo[g]quinoline, benzo[b][1,8]naphthyridine, and anthyridine. The equilibrating step is carried out under conditions effective to form homo- and/or hetero-dimers comprising one solution phase monomer and one substrate-bound monomer, or oligomer molecules comprising more than one solution phase monomer and one substrate-bound monomer. The labeled mRNA regulatory sequence is detected, and the homo- and/or hetero-dimer compounds or oligomer compounds capable of selectively binding the mRNA regulatory sequence are selected based on said detecting.

A ninth aspect of the present invention is directed to a method of altering the activity of a target RNA molecule. This method involves contacting the RNA molecule with a dimer compound according to the second aspect of the present invention that selectively binds to the target RNA molecule or an oligomer compound according to the third aspect of the present invention that selectively binds to the target RNA molecule. The contacting of the compound with the RNA molecule is effective to alter activity of the RNA molecule.

A tenth aspect of the present invention is directed to a method of treating a subject for type I myotonic dystrophy. This method involves administering to a subject a dimer compound according to the second aspect of the present invention, an oligomeric compound according to the third aspect of the present invention, or a composition according to the fourth aspect of the present invention under conditions effective to inhibit (CUG)ₙ repeat RNA-MBNL1 binding in the subject, thereby treating the subject for type I myotonic dystrophy.

An eleventh aspect of the present invention is directed to a method of disrupting the interaction of (CUG)ₙ repeat RNA with MBNL1. This method involves providing a homo- or hetero-dimer compound according to the second aspect of the present invention or an oligomeric compound according to the third aspect of the present invention, and contacting a (CUG)ₙ repeat RNA under conditions effective to inhibit binding of the (CUG)ₙ repeat RNA to MBNL1, thereby disrupting the interaction of (CUG)ₙ repeat RNA with MBNL1.

A twelfth aspect of the present invention is directed to a method of making a homo- or hetero-dimer compound. This method involves providing a first and second monomer having a structure

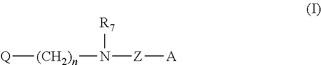

where:
Q is independently selected from H, NH₂,

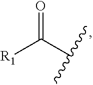

and an inert substrate, where R₁ is selected from a straight or branched chain C₁ to C₆ hydrocarbon and an aromatic or heteroaromatic group;

n is independently an integer from 0 to about 5;

R₇ is selected from hydrogen and an aromatic or heteroaromatic group;

Z is independently a peptide, N-alkylated peptide, or a reduced peptide containing at least two and up to about ten amino acids, where one of the amino acids is capable of forming a disulfide bond, sulfinyl thio linkage, olefin bond, or hydrocarbon bond; and

A is independently hydrogen or an aromatic or heteroaromatic group connected to Z via a carbonyl linkage, provided that in at least one of the first or second monomers A is not hydrogen and is selected from a substituted or unsubstituted benzo[g]quinoline, benzo[b][1,8]naphthyridine, and anthyridine. The first and second monomers are reacted under conditions effective to form a homo- or hetero-dimer compound according to the second aspect of the present invention.

A thirteenth aspect of the present invention is directed to a method of making an oligomeric compound. This method involves providing a first and second monomer having a structure

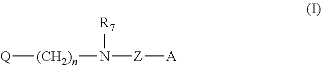

wherein:

Q is selected from H, NH₂,

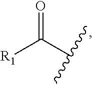

and an inert substrate, wherein R₁ is selected from a straight or branched chain C₁ to C₆ hydrocarbon, NH₂, and an aromatic or heteroaromatic group;

n is an integer from 0 to about 5;

R₇ is selected from hydrogen and an aromatic or heteroaromatic group;

Z is a peptide containing at least two and up to about ten amino acids, where one of the amino acids is capable of forming a bond by hydrazone exchange, a disulfide bond, sulfinyl thio linkage, olefin bond, or hydrocarbon bond; and

A is selected from a substituted or unsubstituted benzo[g]quinoline, benzo[b][1,8]naphthyridine, and anthyridine connected to Z via a carbonyl linkage or A is selected from

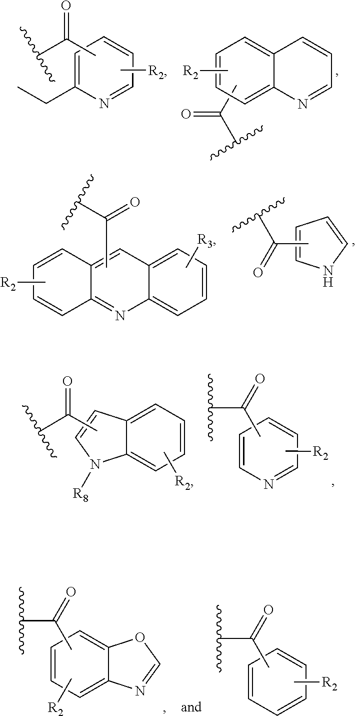

connected to Z via a carbonyl linkage, wherein

R₂ and R₃ are independently selected from the group consisting of hydrogen, methyl, ethyl, propyl, amino, methylamine, ethylamine, dimethylamine, diethylamine, methoxy, ethoxy, propoxy, hydroxyl, cyano, thiocyanato, and aroylhydrazonoalkyl; and

R₈ is H or C₁-C₃ alkyl.

The first and second monomers are reacted under conditions effective to form a homo- or hetero-dimer compound linked by a hydrazone exchange bond, a disulfide bond, sulfinyl thio linkage, olefin bond, or hydrocarbon bond; and then the homo- or hetero-dimer compound is reacted with a third compound having the monomeric structure under conditions effective to form the oligomeric compound, wherein the third compound is linked to the first or second monomer by a hydrazone exchange bond, a disulfide bond, sulfinyl thio linkage, olefin bond, or hydrocarbon bond to form an oligomer of the third aspect of the present invention.

Compounds of the present invention have the ability to bind at least two different RNA targets: the first being an HIV-1 RNA stem/loop frameshift site associated with Gag/Pol expression and the second being an RNA CUG₍ₙ₎ repeat associated with a form of muscular dystrophy (and representative of RNA repeats generally). Relative to the best binding compounds disclosed in PCT Publ. No. WO 2009/015384 to Miller et al., several compounds disclosed herein demonstrate improved binding affinity and selectivity for the target RNA molecules. Because these compounds have demonstrated success in inhibiting the activity of the target nucleic acid molecules, including in an in vivo mouse model of myotonic dystrophy, these compounds and their derivatives should provide effective therapy of the diseases associated with these RNA molecules. These compounds can be used in combination with other known or hereafter developed therapies for these same diseases. Moreover, the compounds encompassed by the present invention should provide a rich resource to identify other compounds capable of binding other target nucleic acid molecules that are associated with particular disease states. For example, ribosomal frameshifting RNA elements are found in a variety of diseases including SARS-CoV (Brierley et al., “Programmed Ribosomal Frameshifting in HIV-1 and SARS-CoV,” Virus Research 119:29-42 (2006), which is hereby incorporated by reference in its entirety), Hepatitis (Xu et al., “Synthesis of a Novel Hepatitis C Virus Protein by Ribosomal Frameshift,” EMBO 20:3840-3848 (2001), which is hereby incorporated by reference in its entirety), Rous Sarcoma Virus (Jacks et al., “Signals for the Ribosomal Frameshifting in the Rous Sarcoma Virus Gag-Pol Region,” Cell 55:447-458 (1998), which is hereby incorporated by reference in its entirety), Human T-Cell Leukemia Virus Type II (Kollmus et al., “The Sequences of and Distance Between Two Cis-Acting Signals Determine the Efficiency of Ribosomal Frameshifting in Human Immunodeficiency Virus Type I and Human T-cell Leukemia Virus Type II in vivo,” J. Virol. 68:6087-6091 (1994), which is hereby incorporated by reference in its entirety), and Coronavirus (Brierley et al., “Characterization of an Efficient Coronavirus Ribosomal Frameshifting Signal: Requirement for an RNA Psuedoknot,” Cell 57:537-547 (1989), which is hereby incorporated by reference in its entirety).

### BRIEF DESCRIPTION OF THE DRAWINGS

FIG. 1 is a schematic illustration of compound 1 (Prior Art; see PCT Publ. No. WO 2009/015384 to Miller et al., which is hereby incorporated by reference in its entirety) identified via RBDCC, and compounds 2-11 of the present invention.

FIGS. 2A-B are sensorgram graphs showing immobilization procedure. FIG. 2A is a representative sensorgram for streptavidin immobilization on a CM5 sensor chip. FIG. 2B is a sensorgram for non-covalent biotin-RNA capture on a streptavidin coated CM5 sensor surface.

FIG. 3 illustrates thermodynamic affinities (apparent K_{D}, nM) and selected stoichiometries (n) as measured by SPR against three CUG repeats for DM1, one CUG repeat for DM2, and three control RNAs. The DM1 CUG repeats are designated “(CUG)₂” (SEQ ID NO: 1), “(CUG)₄” (SEQ ID NO: 2), and “(CUG)₁₀” (SEQ ID NO: 3); the DM2 CUG repeat is designated “(CCUG)₁₀” (SEQ ID NO: 4). The three control sequences are “(CAG)₁₀” (SEQ ID NO: 5), which assess the U versus A differences in the target affinity; mismatched RNA vs. a CUG-CAG duplex (“Duplex”) (SEQ ID NO: 6); and an RNA sequence derived from the gag/pol frameshift-stimulating region of HIV-1 (“HIV-1 FSS”) (SEQ ID NO: 7). Binding constants are reported as an average of two complete titrations±standard deviation. “ND”=Not Determined.

FIG. 4 is a graph showing that an excess of compound 5 provides roughly double the SPR response for a (CUG)₁₀ chip of an equivalent concentration of compound 10, consistent with the two-fold difference in stoichiometry deduced from Scatchard analysis. Note: The Response Units (RU) has been normalized to 100.

FIGS. 5A-F are graphs showing determination of fluorescence quantum yield of 2-ethylbenzo[g]quinoline carboxylic acid. FIG. 5A shows absorption spectrum of compound (2) in methanol. FIG. 5B shows excitation and emission spectra of benzo[g]quinoline in methanol. FIG. 5C shows emission spectrum of compound (2) in methanol. FIG. 5D shows emission spectrum of quinine sulfate in 0.1 N H₂SO₄. FIGS. 5E and 5F are plots of integrated fluorescence intensity versus absorbance for benzo[g]quinoline and quinine sulfate, respectively.

FIGS. 6A-C are representative titration curves as well as emission/excitation spectra for compounds 4 and 5 (FIG. 1). FIG. 6A is a graph showing excitation and emission spectra for compound 4 in HBS-N buffer. FIG. 6B is a set of representative fluorescence emission intensity spectra for titration of unlabeled CUG10 RNA into 1 μM solution of compound 4 in HBS-N buffer. The fluorescence intensity of the compound showed quenching upon addition of the target RNA. FIG. 6C shows plots of change in fluorescence intensity units (ΔFU=Fₒ−Fₙ) against concentration for titration of compounds 3, 4, and 5, fitted with an onsite-binding fit to obtain apparent K_{D} (table at the bottom right). Error-bars are standard deviations for at least two separate titrations.

FIGS. 7A-C are graphs of binding analysis of extended compounds 10 and 11 to (CUG)₁₀ RNA measured by direct monitoring of benzo[g]quinoline fluorescence. FIG. 7A shows excitation and emission spectra for compound 10 in HBS-N buffer pH=7.4. FIG. 7B is a representative emission spectrum showing the quenching of ligand fluorescence with increasing RNA concentration. FIG. 7C shows plots of change in fluorescence intensity against concentration for the titration of compounds 10 and 11. The data were fitted to one-site binding equations to obtain the K_{D} shown in the table at the bottom left.

FIG. 8 is a series of photographs showing that compounds readily penetrate cell membranes and localize in the nucleus. Representative bright field (lower row) and fluorescence (upper row) images are shown for compound 5 in mouse myoblasts.

FIGS. 9A-B are graphs showing binding analysis of compound 11 to (CCUG)₁₀ RNA measured by direct monitoring of benzo[g]quinoline fluorescence. FIG. 9A is a graph of representative emission spectra showing fluorescence quenching with increasing RNA concentration. FIG. 9B shows plots of change in fluorescence intensity against concentration (black squares), fitted to a one-site binding equation to obtain an apparent K_{D} of 73.62±0.75 nM. The error represents an average of two separate titrations.

FIG. 10 shows two sets of photographs showing only a modest ability of benzo[g]quinoline compound 2 to enter cells, and “scrambled” compound 9 being significantly less able to enter cells than compounds 4, 5, or 11. In the top two photographs, mouse myoblasts (C2-12) were incubated 12 h with 125 μM of compound 4. (Exposure=1.5 s) In the bottom two photographs, human fibroblasts were incubated with a mixture of compounds 4 and 5. Localization is shown, especially in the nucleus of cells.

FIG. 11 is a pair of photographs showing mouse myoblast cells incubated 12 h with 125 μM of compound 9. (Exposure=1.5 s)

FIG. 12 is a pair of photographs showing mouse myoblast cells incubated 12 h with 125 μM of compound 11. (Exposure=1.5 s)

FIGS. 13A-B are graphs showing MTT assays that indicate low toxicity of compounds 4 and 5 as a mixture in human fibroblasts (FIG. 13A) or individually in mouse myoblasts (FIG. 13B). The significantly higher toxicities of daunorubicin and mitomycin C are shown for comparison. Error bars indicate standard deviations of 3 replicates of each concentration.

FIGS. 14A-B show that benzo[g]quinoline compound 2 was found to have no toxicity in fibroblasts at concentrations up to 500 μM. FIG. 14A is a pair of photographs showing mouse myoblast cells incubated 12 h with 125 μM of compound 2. (Exposure=1.5 s) Compound 2 was not internalized by cells. FIG. 14B is a graph showing an MTT cell viability assay of 2-ethyl benzo[g]quinoline carboxylic acid (compound 2) using human fibroblast cells.

FIG. 15 is a graph showing compounds that are able to promote expression of a (CUG)₈₀₀-containing luciferase construct in mouse myoblasts. Firefly luciferase activity is plotted as a ratio of the normalized luminescence from cells containing (CUG)₈₀₀ in the 3′-UTR of the luciferase mRNA (C5-14) to normalized luminescence of cells containing no CUG repeats (C1-S). Error bars indicate standard deviations of luminescence from 3 replicate wells.

FIG. 16 is a bar graphs showing total protein content of C1-S (mouse myoblasts incorporating a luciferase construct containing zero CUG repeat RNA) cells treated with various concentrations of compound 4.

FIG. 17 is a bar graph showing total protein content of C₅₋₁₄ (mouse myoblasts incorporating a firefly luciferase with 800 CUG repeats at the 3′-UTR) cells treated with various concentrations of compound 4.

FIGS. 18A-B are a series of graphs showing compounds that improve MBNL1-dependent splicing in vivo. Asterisks (*) indicate statistical significance and the dashed line is provided as a reference to splicing levels for HSA^{LR} mice (n=3) in the absence of compound (control).

FIG. 19 is a schematic illustration showing the HIV-1 Frameshift Stimulating (“FSS”) RNA containing SEQ ID NO: 7.

FIGS. 20A-C show benzo[g]quinoline-containing disulfide dimer compound 17 (FIG. 20A), its visible fluorescence in aqueous solution (FIG. 20B, compared to water alone), and fluorescence emission spectrum (FIG. 20C).

FIGS. 21A-C show that HIV-1 FSS binding compounds penetrate cells and are non-toxic at useful concentrations. FIG. 21A shows the structure of the olefin-linked dimer compounds 18 and 19. FIG. 21B shows false-color fluorescence image of compound 18 in human fibroblasts. Penetration of compound 18 throughout cells is readily apparent. FIG. 21C shows results of MTT assay with compound 18, using mitomycin C as control. Increasing concentrations of mitomycin C result in greater cell death, while cells tolerate compound 18 well.

FIG. 22 is a bar graph showing viral inhibition results (single dose experiments). Compounds 18 and 14 (designated as dimer 5-5 Z in PCT Publ. No. WO 2009/015384 to Miller et al., which is hereby incorporated by reference in its entirety) decrease viral titer by 24-29% at a concentration of 500 nM relative to null control, while 2-ethyl benzo[g]quinoline carboxylic acid (compound 2, FIG. 1) has no effect at this concentration.

FIG. 23 shows compound 18 inhibits pseudo-typed HIV virus formation in a concentration-dependent manner in HEK293T cells. Error bars represent one standard deviation on the mean (n=3).

FIGS. 24A-B are schematic illustrations showing resin-bound dynamic combinatorial library (FIG. 24A) and ternary resin-bound dynamic combinatorial library generation (FIG. 24B).

FIG. 25 is a schematic illustration of combinatorial library members. For solution phase experiments, R═(CH₂)₃NH₂; for resin-bound experiments, R═(CH₂)₅CONH₂.

FIG. 26 is a graph showing a solution phase library constructed from monomer compounds A1, A2, B1, B2, and thiopropanol (green trace); B1, B2, C1, C2, and aniline (red trace); A1, A2, B1, B2, C1, C2, thiopropanol, and aniline (blue trace) after 3 days of equilibration in ammonium acetate buffer (pH 7.4).

FIG. 27 shows resin bound library constructed from A1 and C1 in solution and resin bound B1 after 6 days of equilibration in the presence of aniline at pH 7.4.

FIG. 28 shows resin bound library constructed from A1, A2, C1, C2 in solution and resin bound B1 and B2, after 1 week of equilibration in the presence of aniline at pH 7.4.

FIG. 29 illustrates Scheme 1, which shows the synthesis of intermediate compound 2,2-ethyl benzo[g]quinoline carboxylic acid.

FIG. 30 illustrates Scheme 2, which shows the synthesis of dimers 4 (Z isomer) and 5 (E isomer) using resin-bound monomer compound 3.

FIG. 31 illustrates Scheme 3, which shows the synthesis of dimer control compounds 6 (Z isomer) and 7 (E isomer). Control compounds 6 and 7 lack an effective intercalating group in each of the monomers (i.e., A group in formula I is H).

FIG. 32 illustrates Scheme 4, which shows the synthesis of dimer control compounds 8 and 9 having a scrambled peptide sequence (i.e., Z group in formula I).

FIG. 33 illustrates Scheme 5, which shows the synthesis of the intermediate L-Fmoc-pentenylglycine (c) by asymmetric alkylation of pseudoephedrine glycinamide hydrate. This results in a peptide bearing an alkenyl substituent available for olefin bond formation.

FIG. 34 illustrates Scheme 6, which shows the synthesis of compounds 10 (Z isomer) and 11 (E isomer).

FIG. 35 illustrates Scheme 7, which shows a protocol that can be used to prepare monomers containing N-methyl amino acids in their peptide (Z) group as defined in formula I.

FIG. 36 illustrates Scheme 8, which shows a protocol for the formation of pseudopeptide variants of the monomers.

### DETAILED DESCRIPTION OF THE INVENTION

The present invention relates to a class of monomer and related homo- and hetero-dimer compounds, which can be synthesized according to a number of approaches including as a self-assembled combinatorial library.

The homo- or hetero-dimer compounds of the invention are formed by a disulfide, sulfinyl thio, hydrocarbon or olefin bond, or a bond by hydrazone exchange between two monomers having a structure

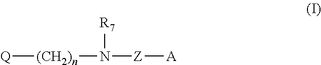

where, for each monomer (I)

Q is independently selected from H, NH₂,

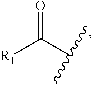

and an inert substrate, where R₁ is selected from a straight or branched chain C₁ to C₆ hydrocarbon and an aromatic or heteroaromatic group;

n is independently an integer from 0 to about 5;

R₇ is selected from hydrogen and an aromatic or heteroaromatic group;

Z is independently a peptide, N-alkylated peptide, or a reduced peptide (pseudopeptide) containing at least two and up to about ten amino acids, where one of the amino acids is capable of forming a disulfide bond, sulfinyl thio linkage, olefin or hydrocarbon bond, or a bond by hydrazone exchange; and

A is independently hydrogen or an aromatic or heteroaromatic group connected to Z via a carbonyl linkage. In certain embodiments, particularly where the bond between peptides groups Z of linked monomers is an olefin, sulfide, or sulfinyl thio, it is provided that in at least one of the monomers A is not hydrogen and is selected from a substituted or unsubstituted benzo[g]quinoline, benzo [b][1,8]naphthyridine, and anthyridine.

Aromatic or heteroaromatic groups A, R₁, and R₇ can be any single, multiple, or fused ring structures, but preferably those that function as intercalator moieties capable of binding to a nucleic acid by inserting itself in between base pairs of adjacent nucleotides with or without unwinding and with or without extension of the nucleic acid helix. Aromatic or heteroaromatic intercalator compounds typically have a flat configuration, and are preferably polycyclic having at least two rings and typically not more than about six rings, more usually not more than about five rings, where at least two of the rings are fused. The rings may be substituted by a wide variety of substituents including, without limitation, alkyl groups of from one to four carbon atoms; oxy groups, which includes hydroxy, alkoxy and carboxy ester, generally of from one to four carbon atoms; amino groups, including mono- and di-substituted amino groups, particularly mono- and dialkyl amino, of from zero to eight, usually zero to six carbon atoms; thio groups, particularly alkylthio from one to four, usually one or two carbon atoms; cyano groups; non-oxo-carbonyl groups, such as carboxy and derivatives thereof, particularly carboxamide or carboxyalkyl, of from one to eight or one to six carbon atoms, usually two to six carbon atoms and more usually two to four carbon atoms; oxo-carbonyl or acyl, generally from one to four carbon atoms; halo groups, particularly of atomic number 9 to 35 (e.g., F, Cl, or Br); or cyclic or heterocyclic compounds (e.g., linked by a zero-carbon bridge).

Specific aromatic or heteroaromatic groups A, R₁, and R₇ (associated carbonyl is shown) include, without limitation:

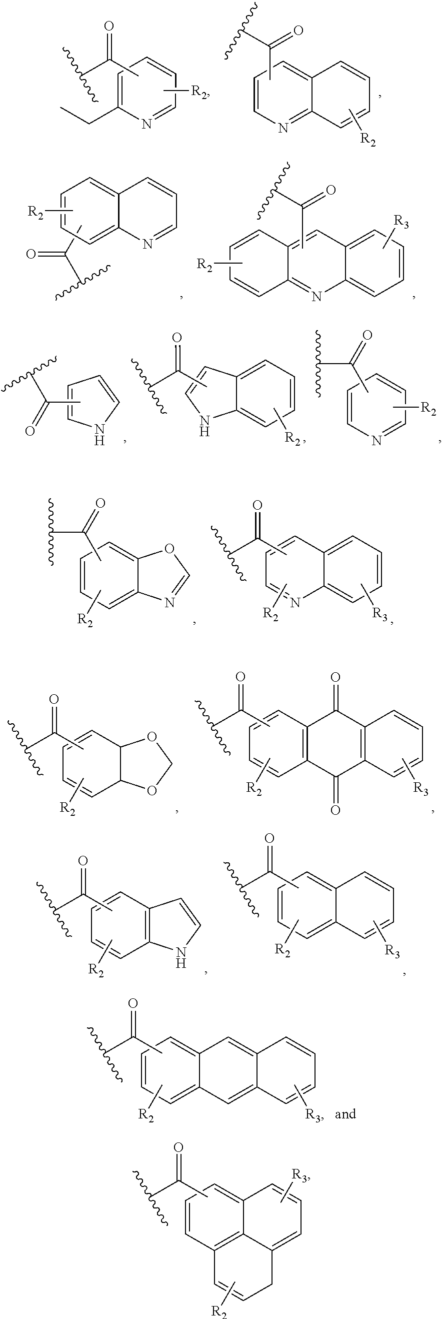

where R₂ and R₃ are optional, and can be any of the substituents identified in the preceding paragraph. Preferably, R₂ and R₃ are independently selected from the group consisting of hydrogen, methyl, ethyl, propyl, amino, methylamine, ethylamine, dimethylamine, diethylamine, methoxy, ethoxy, propoxy, hydroxyl, cyano, thiocyanato, and cyclic or heterocyclic compounds (e.g., linked by a zero-carbon bridge).

As used herein, substituted and unsubstituted benzo[g]quinoline compounds have the structure (associated carbonyl is shown)

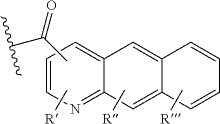

where R′, R″, and R′″ are independently selected from the group consisting of hydrogen, methyl, ethyl, propyl, amino, methylamine, ethylamine, dimethylamine, diethylamine, methoxy, ethoxy, propoxy, hydroxyl, cyano, thiocyanato, and cyclic or heterocyclic compounds (e.g., linked by a zero-carbon bridge).

As used herein, substituted and unsubstituted benzo[b][1,8]naphthyridine compounds have the structure (associated carbonyl is shown)

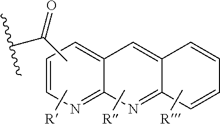

where R′, R″, and R′″ are as defined above.

As used herein, substituted and unsubstituted anthyridine compounds have the structure (associated carbonyl is shown)

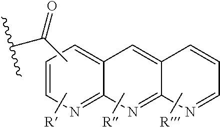

where R′, R″, and R′″ are as defined above.

In peptide Z, the amino acid that is capable of forming a disulfide bond, sulfinyl linkage, hydrocarbon or olefin bond, or a bond by hydrazone exchange, is present at any position in the 2-10 amino acid peptide sequence. Formation of disulfide bonds, sulfinyl linkages, hydrocarbon and olefin bonds is well known in the art. Disulfide bonds are formed by a covalent coupling of thiol groups from a cysteine or cysteine derivative. Sulfinyl linkages can be formed by well-known procedures, either by oxidation of a disulfide bond with mCPBA (Chayajarus et al., “Efficient Synthesis of Carbohydrate Thionolactones,” Tetrahedron Lett. 47:3517-3520 (2006), which is hereby incorporated by reference in its entirety) or by oxidation with dimethyl dioxirane (Bourles et al., “Direct Synthesis of a Thiolato-S and Sulfinato-S Co^{III} Complex Related to the Active Site of Nitrile Hydratase: A Pathway to the Post-Translational Oxidation of the Protein,” Angew. Chem. Int. Ed. 44:6162-6165 (2005), which is hereby incorporated by reference in its entirety). Olefin bonds can be formed by α-amino acids having an unsaturated hydrocarbon sidechain using known procedures, such as those disclosed in PCT Patent Application Publication No. WO 2004/101476, which is hereby incorporated by reference in its entirety.

One method for the formation of a bond by acylhydrazone exchange involves placing an acylhydrazone in a solution in the presence of one or more hydrazides, under conditions favorable for the exchange to occur. Such conditions may involve an acidic medium, or alternatively may be in an ammonium acetate buffer in the presence of an aniline catalyst, as described in Bhat et al., “Nucleophilic Catalysis of Acylhydrazone Equilibration for Protein-directed Dynamic Covalent Chemistry,” Nature Chemistry 2:490-497 (2010), which is hereby incorporated by reference in its entirety. If dual disulfide—hydrazone exchange is desired, this reaction is preferably carried out in ammonium acetate buffer at a pH of approximately 7.4.

In a preferred embodiment, peptide Z is a dipeptide, tripeptide, or tetrapeptide. When peptide Z is a tripeptide, the tripeptide preferably has the structure —R₄—R₅—R₆—; —R₅—R₄—R₆—; —R₅—R₆—R₄—; —R₄—R₆—R₅—; —R₆—R₄—R₅—; or —R₆—R₅—R₄—, where R₄, R₅, and R₆ are amino acids and the amino acid capable of forming a disulfide bond, sulfinyl thio linkage, olefin or hydrocarbon bond, or a bond by hydrazone exchange is R₆.

Any combination of amino acids can be used in the dimer compounds of the present invention including, without limitation, L-amino acids, D-amino acids, and N-methyl amino acids. Preferred amino acids for use in the dimer compound of the present invention include Cys, His, Lys, Phe, Ala, Ser, Asp, Asn, Val, Pro, Thr, Met, Gly, and their derivatives (e.g., allyl-glycine, pentenyl-glycine), as well as their D-amino acids and N-methyl amino acids. N-methylation of amino acids can be achieved with dimethyl sulfate in the presence of sodium hydride and a catalytic amount of water (Prasad et al., “An Efficient and Practical N-Methylation of Amino Acid Derivatives,” Org. Lett. 5(2):125-128 (2003); Holladay et al., “Tetrapeptide CCK-a Agonists: Effect of Backbone N-Methylations on in-vitro and in-vivo CCK Activity,” J. Med. Chem. 37:630-635 (1994); Chatterjee et al., “N-methylation of Peptides: A New Perspective in Medicinal Chemistry,” Acc. Chem. Res. 41:113-1342 (2008), which are hereby incorporated by reference in their entirety).

Inert substrates include, without limitation, resins, glass, thermoplastics, polymer materials, semiconductor materials, and metals. Suitable resins include, without limitation, polystyrene, polystyrene-co-divinylbenzene, and polyethylene glycol/polystyrene-co-divinylbenzene graft polymers. Suitable metals include, without limitation, gold, silver, and platinum. Suitable semiconductor materials include, without limitation, silicon, germanium, doped-silicon alloys, and compound materials such as gallium arsenide and indium phosphide.

In a preferred embodiment, the inert substrate is a resin bead having a diameter of between about 150 μm to about 250 μm.

The present invention also relates to a monomeric compound having a structure

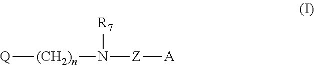

where:

Q is selected from H, NH₂,

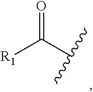

and an inert substrate, where R₁ is selected from a straight or branched chain C₁ to C₆ hydrocarbon, NH₂, and an aromatic or heteroaromatic group;

n is an integer from 0 to about 5;

R₇ is selected from hydrogen and an aromatic or heteroaromatic group;

Z is a peptide containing at least two and up to about ten amino acids, where one of the amino acids is capable of forming a bond by hydrazone exchange, a disulfide bond, sulfinyl thio linkage, olefin bond, or hydrocarbon bond; and

A is selected from a substituted or unsubstituted benzo[g]quinoline, benzo[b][1,8]naphthyridine, and anthyridine connected to Z via a carbonyl linkage or A is selected from

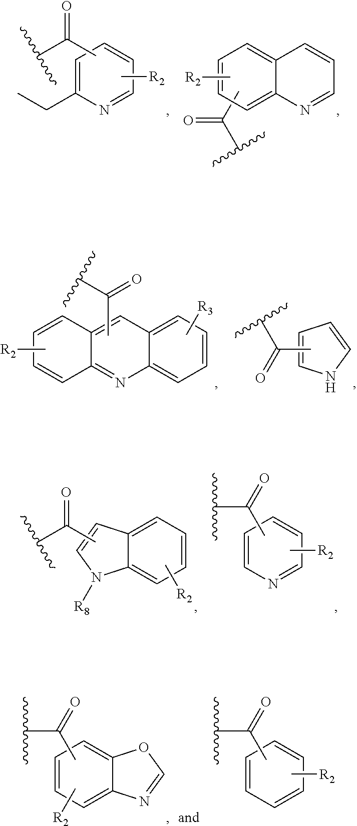

connected to Z via a carbonyl linkage, where R₂ and R₃ are independently selected from the group consisting of hydrogen, methyl, ethyl, propyl, amino, methylamine, ethylamine, dimethylamine, diethylamine, methoxy, ethoxy, propoxy, hydroxyl, cyano, thiocyanato, aroylhydrozonoalkyl, and cyclic or heterocyclic compounds (e.g., linked by a zero-carbon bridge) and R₈ is H or C₁-C₃ alkyl.

The present invention also relates to a method of selecting homo- and/or hetero-dimer compounds capable of selectively binding a target mRNA molecule. The target mRNA molecule can be the full length RNA product that exists in nature, or merely a fragment thereof that possesses the region of interest. In the latter approach, molecular modeling using appropriate software (e.g., RNAStructure) is preferable for determining whether the RNA molecule fragment will retain its shape when part of a minimal structure. Regardless, the target RNA preferably includes a structural- or sequence-specific configuration (i.e., secondary structure) that is targeted by the dimer compounds of the present invention. Preferably, the target mRNA molecule is characterized by a unique stem or stem-loop configuration, and the dimer or oligomer compounds of the present invention specifically target the unique stem or stem-loop structure.

By way of example, the HIV-1 gal-pol mRNA possesses a regulatory sequence containing a stem/loop formation that can be targeted. Another suitable target is the −1 ribosomal frameshifting of SARS coronavirus (Su et al., “An Atypical RNA Pseudoknot Stimulator and an Upstream Attenuation Signal for −1 Ribosomal Frameshifting of SARS Coronavirus,” Nucleic Acids Research 33:4265-4275 (2005); Dos Ramos et al., “Programmed −1 Ribosomal Frameshifting in the SARS Coronavirus,” Biochemical Society Transactions 32:1081-1083 (2004), each of which is hereby incorporated by reference in its entirety). It is expected that any mRNA having a similar frameshift site can be targeted in this manner. Moreover, applicants have demonstrated that the compounds of the present invention can also be used to target other regulatory sequences, such as repeat sequences associated with one or more disease states (e.g., inherited neuropathies, muscular dystrophies, Friedreich ataxia, lysosomal storage diseases, mitochondrial disorders, Huntington's disease, spinocerebellar ataxis (Machado-Joseph disease), dentatorubral pallidoluysian atrophy, and spinobulbar muscular atrophy (Kennedy's disease), fragile X syndrome, Jacobsen syndrome, diabetes mellitus, and myoclonus epilepsy).

Screening for target RNA binding involves providing a heterogeneous mixture of solution phase monomers according to formula (I) each having a structure where Q is independently selected from H, NH₂, and

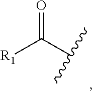

R₁ is selected from a straight or branched chain C₁ to C₆ hydrocarbon and an aromatic or heteroaromatic group, and then equilibrating the heterogeneous mixture of solution phase monomers with a labeled target RNA molecule and a heterogeneous mixture of inert substrate-bound monomers each having a structure of formula (I) except where Q is an inert substrate. At least one of the solution phase or substrate bound monomers A is not hydrogen and is selected from a substituted or unsubstituted benzo[g]quinoline, benzo[b][1,8]naphthyridine, and anthyridine. Inert substrate-bound monomers are preferably covalently attached to the solid support (Q). Methods of attaching compounds to solid supports are well-known in the art and are discussed in PCT Publication No. WO/2009/015384 to Miller et al., which is hereby incorporated by reference in its entirety.

The equilibrating step is carried out under conditions effective to form homo- and/or hetero-dimers containing one solution phase monomer and one substrate-bound monomer. The target mRNA molecule is detected via the label, which becomes bound to the substrate after binding of the RNA molecule by the dimer. Homo- and/or hetero-dimer compounds capable of selectively binding the target RNA molecule are selected (and identified) based on detection of the label in this manner.

Preferably, the RNA target molecule is fluorescently labeled with fluorescent dyes, proteins, or nanocrystalline particles as is well known in the art. Detection of fluorescently labeled compounds can be carried out by methods well-known in the art.

After identifying a particular compound for its binding properties (i.e., for targeting a particular RNA molecule), screening for non-binding to non-target RNA molecules can be carried out using, e.g., total cellular RNA from mammalian cells, yeast, bacteria, plants, etc. This can be used to confirm specificity for the target RNA molecule.

Having thus screened a library of compounds for their ability to bind the target RNA molecule, the dimer compounds of the present invention can be synthesized in substantially pure form. Basically, the synthesis involves providing first and second monomers according to formula (I), which can be the same or different, and then reacting the monomers together under conditions effective to form a homo- or hetero-dimer compound as described above.

In one embodiment, peptide Z of each monomer contains a cysteine residue and the homo- or hetero-dimer compound is formed by a disulfide bond. In yet another embodiment, a dimer compound having a disulfide bond is treated under conditions effective to convert the disulfide bond into a sulfinyl thio linkage.

In an alternative embodiment, peptide Z of each monomer contains an α-amino acid containing an unsaturated hydrocarbon sidechain, such as allylglycine (Al-Gly), and the dimer is formed by an olefin bond. The α-amino acid containing an unsaturated hydrocarbon sidechain may optionally be N-methylated.

According to one embodiment, the dimer compound of the present invention is a homo- or hetero-dimer formed by an olefin bond between two monomers having a structure

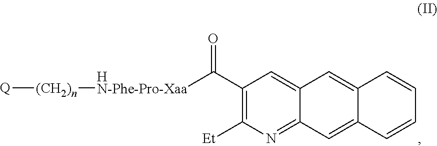

where, for each monomer (II)

Phe is optionally N-methylated;

Xaa is an α-amino acid comprising an unsaturated hydrocarbon sidechain, and is optionally N-methylated;

Q is independently selected from H, NH₂, and an inert substrate; and

n is independently an integer from 0 to about 5.

In another embodiment, the dimer compound of the present invention is a homo- or hetero-dimer formed by an olefin bond between two monomers having a structure

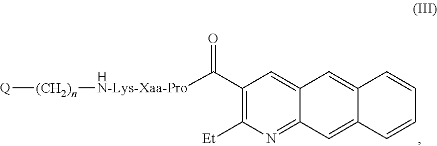

where, for each monomer (III)

Lys is optionally N-methylated;

Xaa is an α-amino acid comprising an unsaturated hydrocarbon sidechain, and is optionally N-methylated;

Q is independently selected from H, NH₂, and an inert substrate; and

n is independently an integer from 0 to about 5.

In yet another embodiment, the dimer compound of the present invention is a homo- or hetero-dimer formed by a disulfide bond between two monomers having a structure

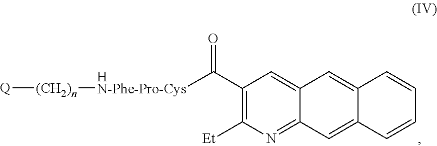

where, for each monomer (IV)

Phe and Cys are optionally N-methylated in one or both of the monomers;

Q is independently selected from H, NH₂, and an inert substrate and

n is independently an integer from 0 to about 5.

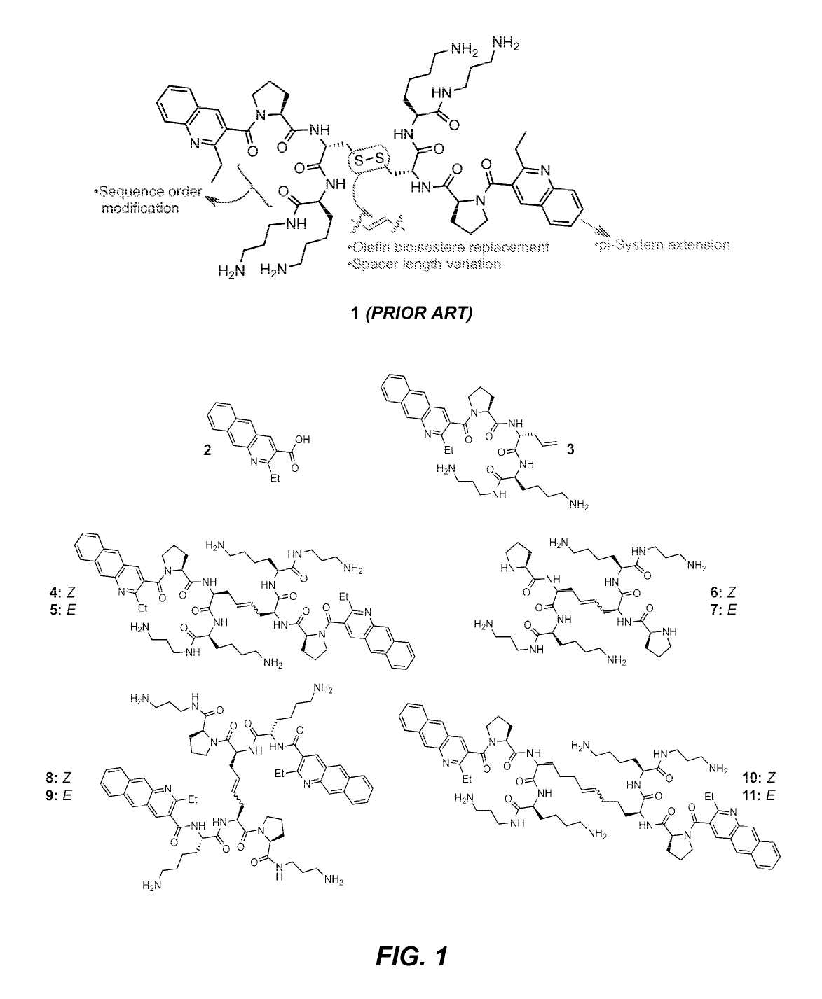

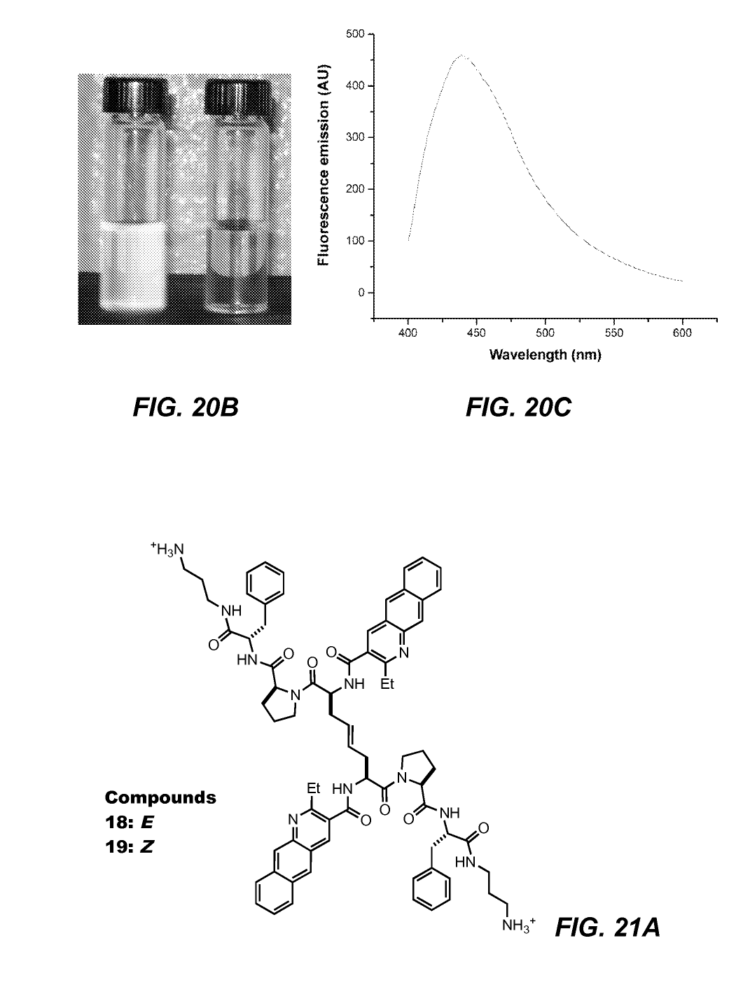

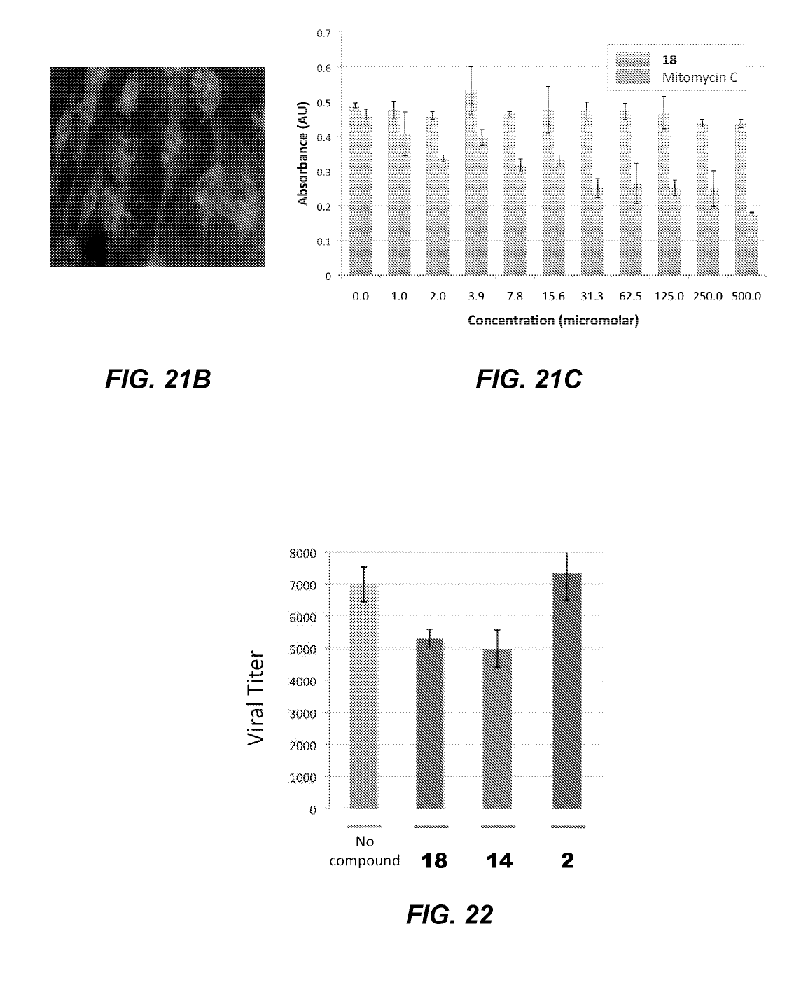

According to another embodiment, dimer compounds of the present invention include compounds 4, 5, 10, and 11 of FIG. 1, compound 17 of FIG. 20A, compounds 18 and 19 of FIG. 21A, and the following N-methyl compounds 20-22:

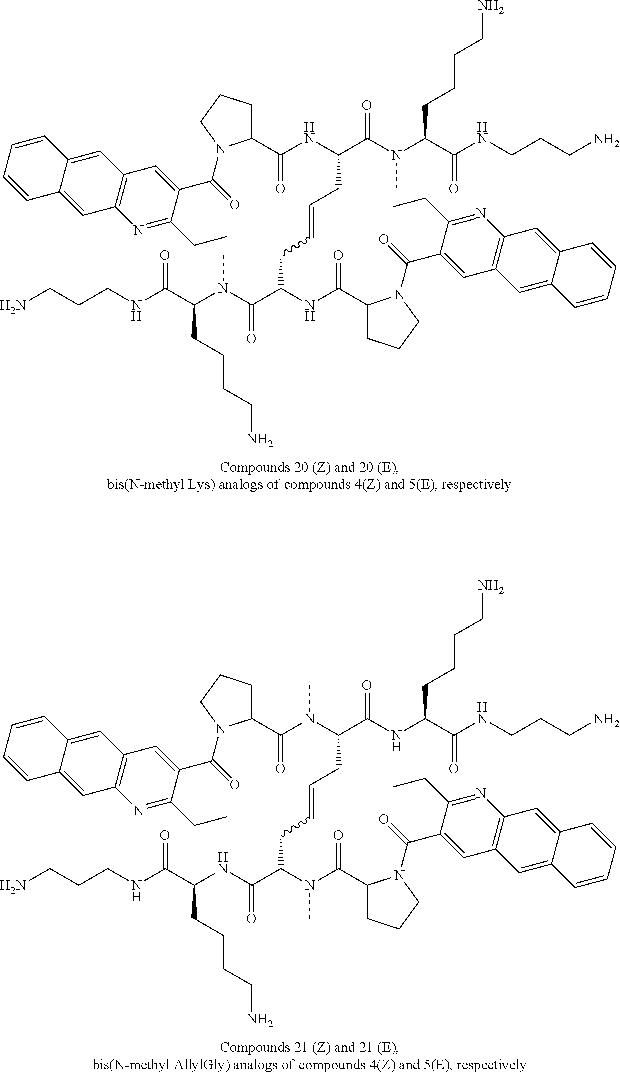

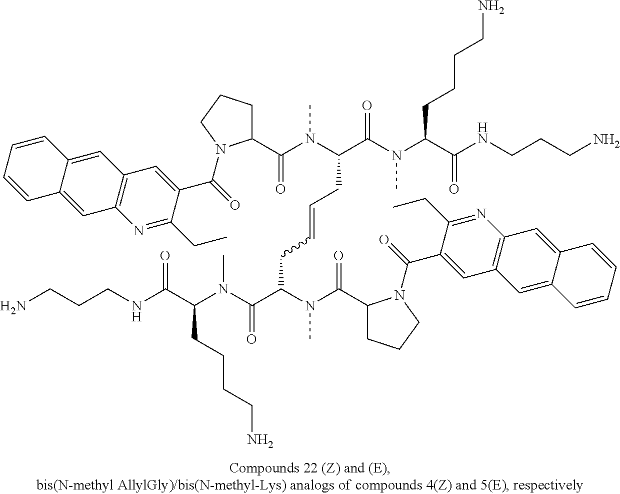

The homo- and hetero-dimers of the present invention can also be linked together to form longer oligomer chains. Oligomerization can be carried out by synthesizing an analog of the cysteine containing peptide in which an allylglycine is incorporated immediately after the diamine linker, followed by gamma-aminobutyric acid or other amino-alkanoic acid. Olefin cross-metathesis following cleavage of the compound from the bead provides the desired oligomer. Oligomerization can also be carried out by synthesizing one analog of the cysteine containing peptide bearing a hydrazone moiety, and another one that contains an aldehyde or its derivative. Hydrazone exchange alone or in combination with disulfide exchange following cleavage of the compound from the bead provides the desired oligomer.

The dimer and oligomer compounds of the present invention can also be in the form of a salt, preferably a pharmaceutically acceptable salt. The term “pharmaceutically acceptable salt” refers to those salts that retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxylic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. Other salts are known to those of skill in the art and can readily be adapted for use in accordance with the present invention.

The dimer and oligomer compounds of the present invention can also be present in the form of a composition that comprises a carrier, preferably a pharmaceutically acceptable carrier. The compositions of the present invention can be in solid or liquid form, such as tablets, capsules, powders, solutions, suspensions, or emulsions.

The dimer and oligomer compounds may be orally administered, for example, with an inert diluent, or with an assimilable edible carrier, or may be enclosed in hard or soft shell capsules, or may be compressed into tablets, or may be incorporated directly with food. For oral therapeutic administration, the compounds of the present invention may be incorporated with excipients and used in the form of tablets, capsules, elixirs, suspensions, syrups, and the like. Such compositions and preparations should contain at least 0.1% of the agent. The percentage of the agent in these compositions may, of course, be varied and may conveniently be between about 2% to about 60% of the weight of the unit. The amount of agent in such therapeutically useful compositions is such that a suitable dosage will be obtained.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar, or both. A syrup may contain, in addition to an active ingredient, sucrose as a sweetening agent, methyl and propylparabens as preservatives, a dye, and flavoring such as cherry or orange flavor.

Compounds of the present invention may also be administered parenterally. Solutions or suspensions of the compounds can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The dimer and oligomer compounds of the present invention may also be administered directly to the airways in the form of an aerosol or other inhalable formulation. For use as aerosols, the agent of the present invention in solution or suspension may be packaged in a pressurized aerosol container together with suitable propellants, for example, hydrocarbon propellants like propane, butane, or isobutane with conventional adjuvants. The agent of the present invention also may be administered in a non-pressurized form such as in a nebulizer or atomizer. An inhalable formulation typically is in the form of an inhalable powder, which may include a pharmaceutically acceptable carrier. Suitable pharmaceutically acceptable carriers for inhalable powders may be composed of any pharmacologically inert material or combination of materials which is acceptable for inhalation. Advantageously, the carrier particles are composed of one or more crystalline sugars; the carrier particles may be composed of one or more sugar alcohols or polyols. In one embodiment, the carrier particles are particles of dextrose or lactose. Conventional dry powder inhalers include the Rotohaler, Diskhaler, and Turbohaler. The particle size of the carrier particles may range from about 10 microns to about 1000 microns. Alternatively, the particle size of the carrier particles may range from about 20 microns to about 120 microns. In certain embodiments, the size of at least 90% by weight of the carrier particles is less than 1000 microns and preferably lies between 60 microns and 1000 microns. The relatively large size of these carrier particles gives good flow and entrainment characteristics. Where present, the amount of carrier particles will generally be up to 95%, for example, up to 90%, advantageously up to 80% and preferably up to 50% by weight based on the total weight of the powder. The amount of any fine excipient material, if present, may be up to 50% and advantageously up to 30%, especially up to 20%, by weight, based on the total weight of the powder.

Sustained release formulations include implantable devices that include a slow-dissolving polymeric matrix and one or more homo- or hetero-dimer compounds retained within the polymeric matrix. The matrix can be designed to deliver substantially the entire payload of the vehicle over a predetermined period of time, such as about one to two weeks up to about one to three months.

Although the formulations and compositions can also be delivered topically, it is also contemplated that the compositions can be delivered by various transdermal drug delivery systems, such as transdermal patches as known in the art.

In addition, the compounds of the present invention can be administered in using a delivery vehicle for passive or targeted delivery to particular cells that are known to possess the target RNA molecule. Any suitable passive or targeted delivery vehicle can be employed, including liposomes, polymeric nanoparticles, polyethylene glycol conjugates, and cell uptake peptides.

Targeting to the delivery vehicle to a cell of interest is typically achieved through the use of antibodies, binding fragments thereof, or nucleic acid aptamers that are bound or suspended to the surface of the delivery vehicle.

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner where the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Wang et al., “pH-sensitive Immunoliposomes Mediate Target-cell-specific Delivery and Controlled Expression of a Foreign Gene in Mouse,” Proc. Natl. Acad. Sci. USA 84:7851 (1987), which is hereby incorporated by reference in its entirety). When liposomes are endocytosed by a target cell, for example, they can be routed to acidic endosomes which will destabilize the liposome and result in drug release.

The liposome delivery system can also be made to accumulate at a target organ, tissue, or cell via active targeting (e.g., by incorporating an antibody or hormone on the surface of the liposomal vehicle). This can be achieved according to known methods.

Different types of liposomes can be prepared according to Bangham et al., “Diffusion of Univalent Ions Across the Lamellae of Swollen Phospholipids,” J. Mol. Biol. 13:238-252 (1965); U.S. Pat. No. 5,653,996 to Hsu et al.; U.S. Pat. No. 5,643,599 to Lee et al.; U.S. Pat. No. 5,885,613 to Holland et al.; U.S. Pat. No. 5,631,237 to Dzau et al.; and U.S. Pat. No. 5,059,421 to Loughrey et al., each of which is hereby incorporated by reference in its entirety.

Polymeric nanoparticles can be targeted to cell-surface markers using aptamers designed using the SELEX procedure (Farokhzad et al., “Targeted Nanoparticle-aptamer Bioconjugates for Cancer Chemotherapy In Vivo,” Proc. Natl. Acad. Sci. USA 103(16):6315-6320 (2006), which is hereby incorporated by reference in its entirety). Nanoparticles and microparticles may comprise a concentrated core of drug that is surrounded by a polymeric shell (nanocapsules) or as a solid or a liquid dispersed throughout a polymer matrix (nanospheres). General methods of preparing nanoparticles and microparticles are described by Soppimath et al., “Biodegradable Polymeric Nanoparticles as Drug Delivery Devices,” J. Control Release 70(1-2):1-20 (2001), which is hereby incorporated by reference in its entirety. Other polymeric delivery vehicles that may be used include block copolymer micelles that comprise a drug containing a hydrophobic core surrounded by a hydrophilic shell; they are generally utilized as carriers for hydrophobic drugs and can be prepared as found in Allen et al., “Colloids and Surfaces,” Biointerfaces 16(1-4):3-27 (1999), which is hereby incorporated by reference in its entirety. Polymer-lipid hybrid systems consist of a polymer nanoparticle surrounded by a lipid monolayer. The polymer particle serves as a cargo space for the incorporation of hydrophobic drugs while the lipid monolayer provides a stabilizing interference between the hydrophobic core and the external aqueous environment. Polymers such as polycaprolactone and poly(D,L-lactide) may be used while the lipid monolayer is typically composed of a mixture of lipids. Suitable methods of preparation are similar to those referenced above for polymer nanoparticles. Derivatized single chain polymers are polymers adapted for covalent linkage of a biologically active agent to form a polymer-drug conjugate. Numerous polymers have been proposed for synthesis of polymer-drug conjugates including polyaminoacids, polysaccharides such as dextrin or dextran, and synthetic polymers such as N-(2-hydroxypropyl)methacrylamide (HPMA) copolymer. Suitable methods of preparation are detailed in Veronese and Morpurgo, “Bioconjugation in Pharmaceutical Chemistry,” IL Farmaco 54(8):497-516 (1999), which is hereby incorporated by reference in its entirety.

By modifying the dimer compounds, the compounds can be administered as a conjugate with a pharmaceutically acceptable water-soluble polymer moiety. By way of example, a polyethylene glycol conjugate is useful to increase the circulating half-life of the dimer compound, and to reduce the immunogenicity of the molecule. Specific PEG conjugates are described in U.S. Patent Application Publ. No. 20060074200 to Daugs et al., which is hereby incorporated by reference in its entirety. Liquid forms, including liposome-encapsulated formulations, are illustrated by injectable solutions and oral suspensions. Exemplary solid forms include capsules, tablets, and controlled-release forms, such as a miniosmotic pump or an implant. Other dosage forms can be devised by those skilled in the art, as shown, for example, by Ansel and Popovich, Pharmaceutical Dosage Forms and Drug Delivery Systems, 5^{th} Edition (Lea & Febiger 1990), Gennaro (ed.), Remington's Pharmaceutical Sciences, 19^{th} Edition (Mack Publishing Company 1995), and by Ranade and Hollinger, Drug Delivery Systems (CRC Press 1996), each of which is hereby incorporated by reference in its entirety.

The dimer and oligomer compounds can be further modified to enhance cellular uptake of the compounds. For example, the dimers and oligomers can be modified with a cell uptake peptide, such as HIV-1 TAT polypeptide or derivative thereof, oligoarginine polypeptide, or Mycobacterium tuberculosis Mce1A polypeptide (22-amino acid sequence termed Inv3), linked to the carboxy-terminal end of the peptide chain (de Coupade et al., “Novel Human-derived Cell-penetrating Peptides for Specific Subcellular Delivery of Therapeutic Biomolecules,” Biochem. J. 390(2):407-418 (2005); U.S. Patent Application Publ. No. 20030032593 to Wender et al.; Wender et al., “The Design, Synthesis, and Evaluation of Molecules that Enable or Enhance Cellular Uptake: Peptoid Molecular Transporters,” Proc. Natl. Acad. Sci. U.S.A. 97:13003-13008 (2000); Brunner et al., “Targeting DNA Mismatches with Rhodium Intercalators Functionalized with a Cell-penetrating Peptide,” Biochemistry 45:12295-12302 (2006); Turner et al., “Synthesis, Cellular Uptake and HIV-1 Tat-dependent Trans-activation Inhibition Activity of Oligonucleotide Analogues Disulphide-conjugated to Cell-penetrating Peptides,” Nucl. Acids Res. 33(1):27-42 (2005); Lu et al., “A Cell-penetrating Peptide Derived from Mammalian Cell Uptake Protein of Mycobacterium tuberculosis,” Anal. Biochem. 353(1):7-14 (2006), each of which is hereby incorporated by reference in its entirety). Thus, in one or more of monomers (I)-(VI) that form the dimer or oligomer, the Q group linked to the carboxy terminus of the peptide chain is replaced by a cell uptake peptide.

As discussed below, and by way of example, several dimer compounds of the present invention are effective in inhibiting the expression of HIV-1 Gag-pol, thereby affording a therapeutic treatment for HIV-1 through the targeting of the Gag-pol mRNA frameshift site. Because HIV-1 infection implicates CD4⁺ T helper cells, macrophages, and dendritic cells, targeted delivery to one or more of these cell types is desirable though not required.

It has been estimated that 39.5 million people are infected with the Human Immunodeficiency Virus (“HIV”) worldwide, 4.3 million of these becoming infected in 2006 alone. Expression and proteolysis of the polyprotein Pol is required for the production of three proteins vital to viral proliferation (HIV-integrase, -protease, and -reverse transcriptase). Pol is produced only as a Gag-Pol fusion protein, which is translated 5-10% with respect to Gag (depending on the technique used for measurement) via a tightly regulated −1 nucleotide ribosomal frameshift (Park et al., “Overexpression of the gag-pol Precursor from Human Immunodeficiency Virus Type 1 Proviral Genomes Results in Efficient Proteolytic Processing in the Absence of Virion Production,” J. Virol. 65:5111-5117 (1991); Jacks et al., “Characterization of Ribosomal Frameshifting in HIV-1 gag-pol Expression,” Nature 331:280-283 (1988); Parkin et al., “Human Immunodeficiency Virus Type 1 gag-pol Frameshifting is Dependent on Downstream mRNA Secondary Structure: Demonstration by Expression in vivo” J. Virol. 66:5147-5151 (1992), each of which is hereby incorporated by reference in its entirety). Two principal factors responsible for this frameshift are (i) a UUUUUUA “slippery sequence” where the frameshift occurs and (ii) a highly conserved downstream stem-loop which has been shown to play a vital role in frameshifting (Telenti et al., “Analysis of Natural Variants of the Human Immunodeficiency Virus Type 1 gag-pol Frameshift Stem-Loop Structure,” J. Virol. 76:7868-7873 (2002), which is hereby incorporated by reference in its entirety). Precise control of frameshifting is essential to viral proliferation, as small changes in Gag-Pol expression levels drastically inhibit virus production (Karacostas et al., “Overexpression of the HIV-1 Gag-Pol Polyprotein Results in Intracellular Activation of HIV-1 Protease and Inhibition of Assembly and Budding of Virus-like Particles,” Virology 193:661-671 (1993); Hung et al., “Importance of Ribosomal Frameshifting for Human Immunodeficiency Virus Type 1 Particle Assembly and Replication,” J. Virol. 72:4819-4824 (1998); Shehu-Xhilaga et al., “Maintenance of the Gag/Gag-Pol Ratio Is Important for Human Immunodeficiency Virus Type 1 RNA Dimerization and Viral Infectivity,” J. Virol. 75:1834-1841 (2001), each of which is hereby incorporated by reference in its entirety).

Another aspect of the present invention is directed to a method of altering the activity of a target RNA molecule. This method involves contacting the RNA molecule with a dimer compound according to the first aspect of the present invention that selectively binds to the target RNA molecule, said contacting being effective to alter activity of the RNA molecule.

Compounds of the present invention have demonstrated affinity for binding selectively to the HIV-1 frameshift regulatory sequence. These compounds are believed to inhibit HIV-1 replication. Thus, a further aspect of the present invention relates to a method of inhibiting HIV-1 proliferation. This method involves providing a dimer or oligomer compound according to the present invention and contacting an HIV-1 mRNA that encodes Pol polypeptide with the dimer or oligomer compound under conditions effective to alter normal expression of the Pol polyprotein and thereby inhibit HIV-1 proliferation.

According to this aspect of the present invention, contacting an HIV-1 mRNA that encodes Pol polypeptide with the dimer or oligomer compound of the present invention may involve contacting an HIV-1 infected cell or (prior to infection) contacting a cell that is targeted by HIV-1 such that the dimer or oligomer compound of the invention is internalized into the cell. Internalization will allow the dimer or oligomer compound to bind an HIV-1 frameshift regulatory sequence which is important to frameshifting of the Gag-pol RNA, which in turn is essential to viral proliferation. For example, small changes in Gag-Pol expression levels in HIV-1 drastically inhibit virus production. Contacting an HIV-1 mRNA that encodes Pol polypeptide with a dimer or oligomer compound of the present invention may be carried out in vitro, such as in a sample, or in vivo in an animal or patient. Thus, this aspect of the present invention can be used to treat blood samples obtained from HIV-1 infected patients.

Another aspect of the present invention relates to a method of treating HIV-1 in a human patient. This method involves administering to a human patient a dimer or oligomer compound according to the first aspect of the present invention under conditions effective to alter normal expression of HIV-1 Pol polyprotein, thereby disrupting HIV-1 proliferation to treat the human patient for HIV-1.

In practicing the methods of treating HIV-1 in a patient of the present invention, the administering step is carried out by administering an agent (i.e., the homo- or hetero-dimer or oligomer compound, or a composition containing the dimer or oligomer compound) orally, intradermally, intramuscularly, intraperitoneally, intravenously, subcutaneously, or intranasally. The agent of the present invention may be administered alone or with suitable pharmaceutical carriers, and can be in solid or liquid form, such as tablets, capsules, powders, solutions, suspensions, or emulsions.

In treating an HIV-1 infected patient, it is intended that the dimer compounds can be used effectively to reduce viral load in a patient or, under certain circumstances, completely eradicate the virus.

The dimer compounds, by virtue of their affinity for binding to the HIV-1 Gag-pol RNA, can also be used for diagnostic screening to detect the presence of HIV-1 virus in a sample. This method involves providing a homo- or hetero-dimer compound according to the first aspect of the present invention, where the dimer compound is immobilized on a surface. The immobilized dimer compound is contacted with a sample under conditions effective to permit the HIV-1 Gag-pol (containing the mRNA frameshift regulatory molecule of HIV-1) to bind specifically to the immobilized homo- or hetero-dimer compound. Presence of the mRNA molecule is detected in the sample based on the binding (i.e., a detectable event). Detection can be achieved using label-free detection schemes like those reported in U.S. Patent Application Publ. No. 2003/0112446 to Miller and Rothberg, which is hereby incorporated by reference in its entirety. Detection can also be achieved using secondary detection labels, such as aptamers or antibodies.

In one embodiment of this aspect of the present invention, the sample is from a blood sample, preferably a human blood sample. Thus, this method of the present invention can be used to detect the presence of HIV-1 in human blood samples.

The surface on which the homo- or hetero-dimer compound of the present invention is immobilized on may be made from a variety of materials and/or types of devices including, without limitation, a silicon-containing chip or a dipstick-like surface which can be inserted into a liquid sample for testing.

Other diseases and/or disorders are also amenable to treatment using the compounds of the present invention.

For example, there are many lines of evidence supporting a toxic RNA mechanism in myotonic dystrophy. Myotonic dystrophy type 1 (MD1) is the most common form of muscular dystrophy in adults, affecting 1 in 8000 people (Machuca-Tzili et al., “Clinical and Molecular Aspects of the Myotonic Dystrophies: A Review,” Muscle Nerve 32:1-18 (2005), which is hereby incorporated by reference in its entirety). DM1 is characterized by multisystemic symptoms, including myotonia, wasting of the muscle, testicular atrophy, cataracts, and cardiac defects. Unlike typical genetic diseases, which follow the traditional central dogma (a mutated gene is transcribed and translated to an altered encoded protein which affects cellular function), DM1 is governed by an RNA mediated mechanism (Wheeler et al., “Myotonic Dystrophy: RNA-mediated Muscle Disease,” Curr. Opin. Neurology 20:572-576 (2007), which is hereby incorporated by reference in its entirety). Specifically, DM1 is caused by expansion of CTG repeats located in the 3′ untranslated region of the DMPK (DM protein kinase) gene on chromosome 19q (Brook et al., “Molecular Basis of Myotonic Dystrophy: Expansion of a Trinucleotide (CTG) Repeat at the 3′ End of a Transcript Encoding a Protein Kinase Family Member,” Cell 68:799-808 (1992), which is hereby incorporated by reference in its entirety). Transcription produces toxic mRNA containing hundreds to thousands of (CUG) repeats, which form long and stable hairpin structures (Michalowski et al., “Visualization of Double-stranded RNAs from the Myotonic Dystrophy Protein Kinase Gene and Interactions with CUG-binding Protein,” J. Nucleic Acids Res. 27:3534-42 (1999), which is hereby incorporated by reference in its entirety). The (CUG) repeat RNA accumulates in nuclear foci, and sequesters RNA binding proteins such as the MBNL (muscleblind) family of splicing regulators (Lin et al., “Failure of MBNL1-dependent Post-natal Splicing Transitions in Myotonic Dystrophy,” Hum. Mol. Genet. 15:2087-2097 (2006), which is hereby incorporated by reference in its entirety). (CUG) repeat sequestration of these splicing regulators causes misregulated and aberrant splicing of a variety of gene products, including the chloride channel 1, which is a major cause of myotonia in DM (Mankodi et al., “Expanded CUG Repeats Trigger Aberrant Splicing of C1C-1 Chloride Channel Pre-mRNA and Hyperexcitability of Skeletal Muscle in Myotonic Dystrophy,” Molecular Cell 10:35-44 (2002); Wheeler et al., “Correction of C1C-1 Splicing Eliminates Chloride Channelopathy and Myotonia in Mouse Models of Myotonic Dystrophy,” J. Clin. Invest. 117:3952-3957 (2007), each of which is hereby incorporated by reference in its entirety). As such, an RNA mediated model of DM1 pathogenesis has been established.

The expanded (CUG)ₙ or (CCUG)ₙ repeat RNA of DM1 and DM2 function in pathogenesis by causing misregulated and aberrant splicing. The (CUG)ₙ repeat RNA accumulates in the nucleus and interacts with CUG binding proteins. These CUG binding proteins such as CELFs (CUG binding proteins and ETR3 like factors) and MBNLs (muscleblind) are regulators of splicing. CELF proteins, in particular CUGBP1 (CUG binding protein 1) show activity increase in myotonic dystrophy (Timchenko et al., “Identification of a (CUG)ₙ Triplet Repeat RNA-binding Protein and Its Expression in Myotonic Dystrophy,” Nucleic Acids Research 24:4407-4414 (1996); Timchenko et al., “RNA CUG Repeats Sequester CUGBP1 and Alter Protein Levels and Activity of CUGBP1,” Journal of Biological Chemistry 276:7820-7826 (2001), each of which is hereby incorporated by reference in its entirety). The (CUG)ₙ RNA forms nuclear foci, or inclusions, in muscle cells (Taneja et al., “Foci of Trinucleotide Repeat Transcripts in Nuclei of Myotonic Dystrophy Cells and Tissues,” Journal of Cell Biology 128:995-1002 (1995), which is hereby incorporated by reference in its entirety) and muscleblind (MBNL) proteins such as MBNL1 are sequestered to these foci (Jiang et al., “Myotonic Dystrophy Type 1 Associated with Nuclear Foci of Mutant RNA, Sequestration of Muscleblind Proteins, and Deregulated Alternative Splicing in Human Neurons,” Human Molecular Genetics 12:3079-3088 (2004); Mankodi et al., “Muscleblind Localizes to Nuclear Foci of Aberrant RNA in Myotonic Dystrophy Types 1 and 2,” Human Molecular Genetics 10:2165-2170 (2001), each of which is hereby incorporated by reference in its entirety). The muscleblind and CELF proteins control developmentally programmed mRNA processing. In the regulation of splicing, MBNL proteins antagonize CELF proteins activities. When CELF protein activity dominates, splicing follows an embryonic pattern. Alternatively, when MBNL activity dominates, splicing follows an adult pattern. Specifically, MBNL 1 promotes and regulates alternative exon inclusion in muscle differentiation (Pascual et al., “The Muscleblind Family of Proteins: An Emerging Class of Regulators of Developmentally Programmed Alternative Splicing,” Differentiation 74:65-80 (2006), which is hereby incorporated by reference in its entirety). Thus, the sequestration of MBNL proteins in DM1 leads to an imbalance in the MBNL/CELF activity ratio, thereby causing misregulation of mRNA processing. Importantly, it has been shown in mouse models that (CUG)ₙ repeat expression or MBNL1 ablation both result in similar splicing defects as seen in human DM1 (Lin et al., “Failure of MBNL1-dependent Postnatal Splicing Transitions in Myotonic Dystrophy,” Human Molecular Genetics 15:2087-2097 (2006), which is hereby incorporated by reference in its entirety). Additionally, the altered spliceopathy seen in long (CUG). RNA repeat expressing muscle cells can be reversed by MBNL1 overexpression (Kanadia et al., “Reversal of RNA Missplicing and Myotonia After Muscleblind Overexpression in a Mouse Poly(CUG) Model for Myotonic Dystrophy,” PNAS U.S.A. 103:11748-11753 (2006), which is hereby incorporated by reference in its entirety). As such, small molecules capable of binding (CUG) repeat RNA and disrupting its interaction with splicing proteins are highly desirable as potential therapeutic agents to restore normal splicing in DM1.

The altered spliceopothy associated with DM1 affects many gene products (Table 1) (Ranum et al., “RNA-mediated Neuromuscular Disorders,” Annual Review of Neuroscience 29:259-277 (2006), which is hereby incorporated by reference in its entirety). Some of the most prevalent include the altered splice product of the insulin receptor which causes insulin resistance in the DM patients. Also, it has been shown that defects in the splicing of chloride channel 1 (C1C1) are responsible for myotonia in DM1 (Mankodi et al., “Expanded CUG Repeats Trigger Aberrant Splicing of C1C-1 Chloride Channel Pre-mRNA and Hyperexcitability of Skeletal Muscle in Myotonic Dystrophy,” Molecular Cell 20:35-44 (2002); Wheeler et al., “Correction of C1C-1 Splicing Eliminates Chloride Channelopathy and Myotonia in Mouse Models of Myotonic Dystrophy,” Journal of Clinical Investigation 117:3952-3957 (2007), each of which is hereby incorporated by reference in its entirety). In addition, altered splicing of cardiac troponin T leads to cardiac abnormalities. In the brain, Tau, APP (amyloid precursor protein), and NMDAR-1 (N-methyl-D-aspartate receptor) are alternatively spliced and this process has been hypothesized to lead to mental retardation commonly associated with DM. Finally, a variety of altered splice products including MTMR1 (myotubularin-related protein 1) and RyR (ryanodine receptor) are associated with muscle wasting (Ranum et al., “RNA-mediated Neuromuscular Disorders,” Annual Review of Neuroscience 29:259-277 (2006); Osborne et al., “RNA-dominant Diseases,” Human Molecular Genetics 15(Review 2):R162-R169 (2006), each of which is hereby incorporated by reference in its entirety).

TABLE 1
Genes Identified to be Alternatively Spliced in DM1,
their location, and the exons or introns affected

| Tissue | Gene | Target |
|---|---|---|
| Skeletal Muscle | ALP | Ex 5a, 5b |
| | CAPN3 | Ex 16 |
| | CLCN1 | Int 2, ex 7a, 8a |
| | FHOS | Ex 11a |
| | GFATI | Ex 10 |
| | IR | Ex 11 |
| | MBNL1 | Ex 7 |
| | MBNL2 | Ex 7 |
| | NRAP | Ex 12 |
| | MTMR1 | Ex 2.1, 2.2 |
| | RYR1 | Ex 70 |
| | z-titin | Ex Zr4, Zr5 |
| | m-titin | M-line ex 5 |
| | ZASP | Ex 11 |
| | SERCA1 | Ex 22 |
| | SERCA2 | Int 19 |
| Heart | ALP | Ex 5 |
| | TNNT2 | Ex 5 |
| | ZASP | Ex 11 |
| | m-titin | M-line ex 5 |
| | KCNAB1 | Ex 2 |
| Brain | TAU | Ex 2, 10 |
| | APP | Ex 7 |
| | NMDAR1 | Ex 5 |

To date, alternative splicing of these genes has been attributed to myotonia, muscle wasting, insulin resistance, cardiac defects and mental problems.

Thus, another aspect of the present invention is directed to a method of treating a subject for type I myotonic dystrophy. This method involves administering to a subject a homo- or hetero-dimer compound according to the first aspect of the present invention under conditions effective to inhibit (CUG)ₙ repeat RNA-MBNL1 binding in the subject, thereby treating the subject for type I myotonic dystrophy.

Another aspect of the present invention is directed to a method of disrupting the interaction of (CUG)ₙ repeat RNA with MBNL1. This method involves providing a homo- or hetero-dimer compound according to the first aspect of the present invention and contacting a (CUG)ₙ repeat RNA under conditions effective to inhibit binding of the (CUG)ₙ repeat RNA to MBNL1, thereby disrupting the interaction of (CUG)ₙ repeat RNA with MBNL1.

In addition to muscular dystrophy, other unstable noncoding expanded repeats are commonly associated with neurological and muscular disease (Table 2) (Machuca-Tzili et al., “Clinical and Molecular Aspects of the Myotonic Dystrophies: A Review,” Muscle Nerve 32:1-18 (2005), which is hereby incorporated by reference in its entirety).

TABLE 2
Common Diseases Associated with Noncoding Expanded Repeat
Sequences

| Disease | Repeat | Normal (n) | Disease (n) |
|---|---|---|---|
| Myotonic Dystrophy 1 | (CTG)ₙ | 5-37 | 50 -> 2000 |
| Friedreichs Ataxia | (GAA)ₙ | 6-32 | 200-1700 |
| Spinocerebellar Ataxia 8 | (CTG)ₙ | 16-92 | >100 |
| Spinocerebellar Ataxia 12 | (CAG)ₙ | 7-45 | 55-80 |
| Fragile X Syndrome | (CGG)ₙ | 4-50 | >200 |
| Jacobsen Syndrome | (CCG)ₙ | 11 | >100 |
| Myotonic Dystrophy 2 | (CCTG)ₙ | 104-176 | 75-11000 |
| Diabetes Mellitus | (ACAGGGGT(G/C))ₙ | 110-150 | 30-44 |
| | SEQ ID NO: 12 | | |
| Myoclonus Epilepsy | (CCCCGCCCCGCG)ₙ | 2-3 | 30-75 |
| | SEQ ID NO: 13 | | |
| Spinocerebellar Ataxia 10 | (ATTCT)ₙ | 10-22 | 800-4500 |
| Familial ALS* | (GGGGCC)ₙ | | 9 -> 30 |

*See Renton et al., “Expanded GGGGCC Hexanucleotide Repeat in Noncoding Region of C9ORF72 Causes Chromosome 9p-Linked FTD and ALS,” Neuron 72: 257-268 (2011) and Dejesus-Hernandez et al., “A Hexanucleotide Repeat Expansion in C9ORF72 Is the Cause of Chromosome 9p21-Linked ALS-FTD,” Neuron 72: 245-256 (2011), which are hereby incorporated by reference in their entirety.

Based on the results demonstrated in the following Examples, compounds of the present invention have demonstrated affinity for binding selectively to expanded repeat RNA sequences. These compounds can be used to interfere with the sequestration of splicing proteins by these expanded repeat RNA sequences. Thus, a further aspect of the present invention relates to a method of interfering with the interaction between an expanded repeat RNA sequence and a splicing protein. This method is carried out by contacting the expanded repeat RNA sequence with a dimer or oligomer compound of the present invention under conditions effective to prevent splicing protein sequestration by the expanded repeat RNA sequence. It is intended that compounds of the present invention can reduce the total amount of splicing protein that is sequestered by these repeat sequences, and thereby inhibit formation of dangerous foci.

Another aspect of the present invention relates to a method of treating a disease or disorder associated with expanded repeat RNA sequences. This method involves providing a dimer or oligomer compound according to the present invention, and administering the compound to a patient, preferably a mammal such as a human, under conditions effective to alter function of an expanded repeat RNA sequence, thereby disrupting interaction between the RNA repeat sequence and splicing proteins to treat the subject for the disorder. As used herein, treatment can include stopping or reversing progression of the disease or disorder, or controlling symptoms thereof.

The appropriate dose regimen, the amount of each dose administered, and specific intervals between doses of the active compound will depend upon the particular active compound employed, the conditions of the patient being treated, and the nature and severity of the disorder or conditions being treated. Preferably, the active compound is administered in an amount and at an interval that results in the desired treatment of or improvement in the disorder or condition being treated.

As one skilled in the art will readily appreciate, the compounds of the present invention can be used alone or in combination with other treatments of expanded repeat disorders as a combination therapy.

Compounds of the present invention, particularly those containing a benzo[g]quinoline structure, may be used as diagnostic tools or fluorescence imaging tools. In one embodiment, the compounds are useful to visualize (CUG) foci in cells based on their fluorescent properties.

### EXAMPLES

The following examples are provided to illustrate embodiments of the present invention but are by no means intended to limit its scope.

RBDCC hit compound 1 (FIG. 1) and related molecules identified in initial work (PCT Publ. No. WO 2009/015384, which is hereby incorporated by reference in its entirety) provided a useful demonstration of feasibility, and set the stage for building a compound that possessed higher affinity and would be suitable for further evaluation in the biological context. To accomplish that goal, it was believed that replacing the disulfide bridge with an olefin bioisostere would not have a dramatic impact on affinity, based on results from parallel efforts in targeting an RNA sequence involved in regulating −1 ribosomal frameshifting in HIV (Palde et al., “Strategies for Recognition of Stem-loop RNA Structures by Synthetic Ligands: Application to the HIV-1 Frameshift Stimulatory Sequence,” J. Med. Chem. 53:6018-6027 (2010), which is hereby incorporated by reference in its entirety). Since disulfides are easily reduced in the cytoplasm, replacing the disulfide with an olefin or alkane would facilitate cellular studies. Second, molecules containing hydrocarbon bridges of varied length would allow for examination of the effect of linker length and configuration on binding ability and selectivity. Third, it was desired to explicitly examine the importance of the amino acid sequence order for specific RNA targets. Finally, as quinolines are known intercalators, at least in the DNA-binding context (Varvaresou et al., “Molecular Modeling Study of Intercalation Complexes of Tricyclic Carboxamides with d(CCGGCGCCGG)₂ and d(CGCGAATTCGCG)₂,” J. Mol. Model. 17:2041-2050 (2011), which is hereby incorporated by reference in its entirety), it was hypothesized that increasing the pi surface area of this group would enhance affinity. In this regard, it was surprising to discover that despite the vast amount of research conducted into the nucleic acid recognition properties and biological activity of acridine derivatives, including the use of several acridines in humans as antimicrobials (Wainwright, “Acridine-a Neglected Antibacterial Chromophore,” J. Antimicrob. Chemother. 47:1-13 (2001), which is hereby incorporated by reference in its entirety) and chemotherapeutic agents (Horstmann et al., “Amsacrine Combined with Etoposide and High-dose Methylprednisolone as Salvage Therapy in Acute Lymphoblastic Leukemia in Children,” Haematologica 90:1701-1703 (2005), which is hereby incorporated by reference in its entirety), there is only one mention of the closely related benzo[g]quinoline heterocycle (i.e., compound 2, FIG. 1) in the nucleic acid recognition literature (Petrov et al., “In Vitro Interaction of Chick Erythrocyte DNA with Substituted 4-amino Benzo[g]quinolines,” Khim.-Farmat. Zhurnal 16:1304-1306 (1982), which is hereby incorporated by reference in its entirety). Thus, synthesizing and testing derivatives incorporating this moiety would constitute the first examination of this heterocycle in the RNA binding context.

In the synthesis schemes described below, commercially available reagents were obtained from Sigma Aldrich Chemical Co. (St. Louis, Mo.), TCI America (Portland, Oreg.), Fisher Scientific, EMD Chemicals (Gibbstown, N.J.), Advanced ChemTech (Louisville, Ky.), and Alfa Aesar (Ward Hill, Mass.) and were used without further purification unless otherwise noted. Water used for reactions and aqueous workup was glass-distilled from a deionized water feed. Reagent grade solvents were used for all non-aqueous extractions. Reaction progress was monitored by analytical thin-layer chromatography (“TLC”) using EM silica gel 60 F-254 precoated glass plates (0.25 mm). Compounds were visualized on the TLC plates with a UV lamp (dual wavelength; λ=254 nm, λ=360 nm). Synthesized compounds were purified using flash column chromatography on EM silica gel 60 (230-400) mesh or alternatively via preparative reversed phase HPLC. Cells were cultured in Dulbecco's modified Eagle's medium (DMEM (GIBCO Cat #11995)), supplemented with 10% FBS and 1% pen-strep. MTT used for viability studies was purchased from CHEMICON, Inc.

Compounds were analyzed by NMR, FT-IR, and low- or high-resolution mass spectra (“HRMS”). ¹H NMR spectra were recorded at 25° C. on either a Bruker Avance 400 (400 MHz) or Bruker Avance 500 (500 MHz) instrument and processed using MestReNova NMR processing software. Chemical shifts (δ) are reported in parts per million (ppm) downfield from tetramethylsilane and referenced to the residual protium signal in the NMR solvents (D₂O, δ=4.79). Data are reported as follows: chemical shift, multiplicity (s=singlet, d=doublet, t=triplet, m=multiplet and q=quartet), coupling constant (J) in Hertz (Hz) and integration. ¹³C spectra were recorded at 25° C. on a Bruker Avance 500 instrument operating at 126 MHz. Chemical shifts (δ) are reported in ppm downfield from tetramethylsilane and referenced (except in D₂O) to the primary carbon resonance in the NMR solvent. FT-IR spectra were recorded on Shimadzu FT-IR spectrophotometer. HRMS were acquired at the university of Buffalo chemistry department mass spectrometry facility, Buffalo, N.Y. or at the mass spectrometry facility of the University of California, Riverside. Low Resolution mass spectra were recorded on Shimadzu LC/MS 2010 with APCI or Electrospray ionization.

### Example 1

### Synthesis of 2-Ethyl Benzo[g]quinoline Carboxylic Acid (Compound 2) from Commercially Available Acrolein

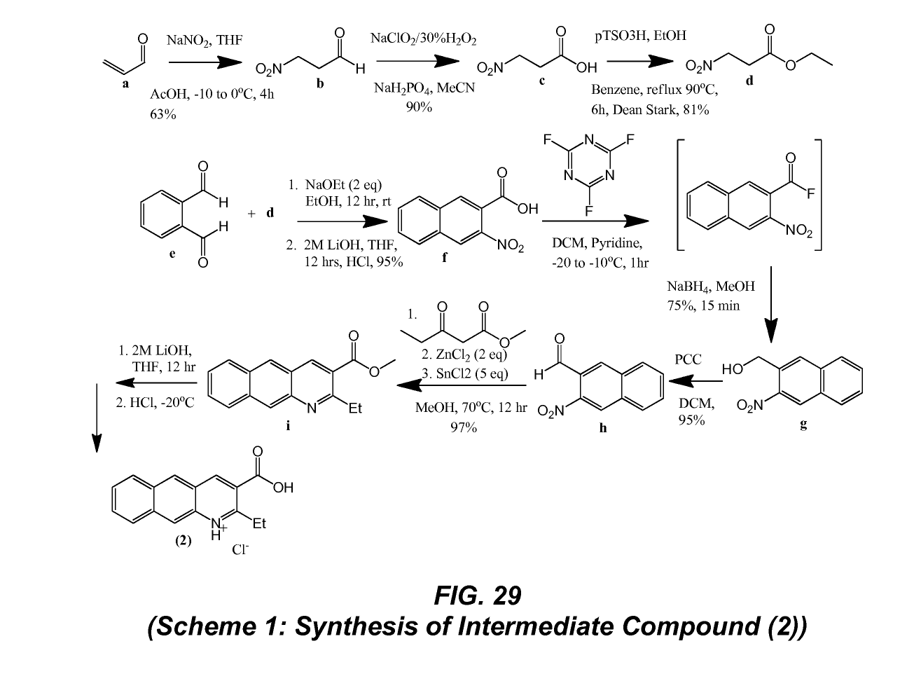

Scheme 1, shown in FIG. 29, was used to synthesize intermediate compound (2), 2-ethyl benzo[g]quinoline carboxylic acid.

Ethyl-3-nitropropanoate (Scheme 1, d) was prepared by following literature procedure (Silva et al., “An Expeditious Synthesis of 3-Nitropropionic Acid and its Ethyl and Methyl Esters,” Synthetic Communications 31:595-600 (2001), which is hereby incorporated by reference in its entirety) starting from commercially available acrolein (Scheme 1, a). Spectral data were comparable to that reported in the literature. Ethyl-3-nitropropanoate (Scheme 1, d): ¹H NMR (400 MHz, CDCl₃) δ: 4.62-4.39 (m, 2H), 4.03 (q, J=7.1 Hz, 2H), 2.92-2.69 (m, 2H), 1.12 (t, J=7.1 Hz, 3H). ¹³C NMR (126 MHz, CDCl₃) δ: 169.64, 69.72, 61.22, 30.84, and 13.82.

3-Nitro-2-naphthoic acid (Scheme 1, f) was synthesized by reacting O-phthaldialdehyde (Scheme 1, e) with ethyl-3-nitropropionate using a method reported by Kienzle, “The Reaction of Phthalaldehydes with 3-nitropropionates. A Simple Route to 3-nitro-2-Naphthoic Acids,” Helv. Chim. Acta 63:2364-2369 (1980), which is hereby incorporated by reference in its entirety. Spectral data were comparable to that reported in the literature. ¹H NMR (400 MHz, CD₃OD) δ: 8.38 (s, 1H), 8.30 (s, 1H), 8.01 (dd, J=8.1, 4.6 Hz, 2H), 7.74-7.64 (m, 3H). ¹³C NMR (126 MHz, CD₃OD) δ: 167.80, 146.75, 134.17, 133.47, 131.89, 130.36, 130.12, 129.61, 129.29, 124.94, 124.87. LRMS (ES−) calculated for C₁₁H₆NO₄ (M−H)^{˜}216 found 216.

(3-Nitronaphthalen-2-yl) methanol (Scheme 1, g) was prepared from (Scheme 1, f) via a one-pot procedure for the conversion of carboxylic acids into alcohols (Kokotos and Noula, “Selective One-Pot Conversion of Carboxylic Acids into Alcohols,” J. Org. Chem. 61:6994-6996 (1996), which is hereby incorporated by reference in its entirety). Briefly, an oven dried two-neck round bottom flask equipped with a mechanical stirrer and a thermometer was cooled under N₂ gas. Pyridine (1.113 mL, 13.8 mmol) was added to a stirred solution of 3-nitro-2-naphthoic acid (3 g, 13.81 mmol) in dry CH₂Cl₂. The flask was cooled to −10 to −20° C. in an acetone/dry ice bath, and then cyanuric fluoride (3.73 g, 27.63 mmol) was added in one portion. The mixture was then stirred vigorously at −10 to −20° C. for 1 hour, diluted with CH₂Cl₂ and then 50 mL of ice-cold water was added. The aqueous phase was extracted (1×100 mL) with CH₂Cl₂ and dried over MgSO₄. The solvent was concentrated under vacuum to a small volume (40 mL) in CH₂Cl₂, and then solid NaBH₄ (1.0451 g, 27.63 mmol) was added in one portion. Methanol (20 mL) was added in drops for over 15 minutes at ambient temperature. The reaction was quenched by addition of 50 mL 1 N aqueous H₂SO₄. The organic solvent was removed under vacuum and the aqueous phase diluted and extracted (2×40 mL) with ethyl acetate. The product was purified by flash column chromatography (silica gel) to yield 2.2 g (81%) of (Scheme 1, g) as a bright yellow compound. ¹H NMR (400 MHz, CD₃OD) δ: 8.56 (s, 1H), 8.14 (s, 1H), 7.94 (dd, J=24.5, 8.1 Hz, 2H), 7.61 (dt, J=28.6, 7.1 Hz, 2H), 5.01 (s, 2H). ¹³C NMR (126 MHz, CD₃OD) δ: 147.14, 136.41, 134.23, 132.55, 130.67, 130.22, 129.17, 128.87, 128.70, 126.50, 62.41. LRMS (ES+) calculated for C₁₁H₁₀NO₄ [M+H]⁺: 204 found: 204.

3-nitro-2-naphthaldehyde (Scheme 1, h): (3-nitronaphthalen-2-yl) methanol (3 g, 14.76 mmol) dissolved in 40 mL of dry CH₂Cl₂ was added slowly to a stirred solution of pyridinium cholorochromate (4.77 g, 22.15 mmol) and celite (4.77 g) in CH₂Cl₂ under N₂ atmosphere at room temperature. The reaction was monitored by TLC until none of the starting material remained, and then diluted with 50 mL of ethyl acetate and filtered through a pad of Florisil. The filtrate was concentrated under vacuum and the product was purified by column chromatography using 20:80 ethyl acetate:hexane to yield 2.8 g (99%) of a yellow crystalline compound. ¹H NMR (400 MHz, CDCl₃) δ: 10.46 (s, 1H), 8.62 (s, 1H), 8.41 (s, 1H), 8.05 (dd, J=8.5, 5.0 Hz, 2H), 7.76 (dd, J=6.2, 3.2 Hz, 2H). ¹³C NMR (126 MHz, CDCl₃) δ: 188.19, 145.77, 134.18, 133.59, 132.09, 130.62, 130.47, 129.78, 129.62, 127.88, 125.97. LRMS (ES+) calculated for C₁₁H₇NO₃ [M+H]⁺: 202 found: 202.

2-ethylbenzo[g]quinoline-3-carboxylic acid (Scheme 1, 2): A flame dried 100 mL 3-neck round bottom flask was equipped with a teflon-coated stir bar, thermometer, and a reflux condenser with N₂ inlet. The flask was charged with 1 g of 3 A molecular sieves, 3-nitro-2-naphthaldehyde (1.5 g, 7.45 mmol), methyl propionylacetate (0.97 g, 7.45 mmol) and ZnCl₂ (2.03 g, 14.9 mmol). Anhydrous methanol (50 mL) was added, flushed with a stream of N₂ and heated while stirring to an internal temperature of 70° C. for 1 hour. Next, SnCl₂ (7.1 g, 37.25 mmol) was added slowly in 3 portions, after which the reaction was stirred at 70° C. and allowed to reflux for 12 hours. After cooling to room temperature, the reaction was made alkaline by the addition of 50 mL K₂CO₃ (10 g dissolved in 50 mL of water) solution. Diethyl ether (50 mL) was added, and the mixture was then filtered through a pad of celite. The celite was washed (3×30 mL) with ether and the combined organics washed (3×50 mL) with brine and concentrated under vacuum to yield a red oily residue. This crude product was dissolved in 10 mL of THF and 40 mL of 2 M aqueous LiOH was added then stirred overnight at room temperature. After removal of the organic solvent under vacuum, the aqueous layer was chilled at −20° C. for 4 hours and then acidified to pH=1 with concentrated HCl. The precipitate formed was filtered, washed with 50 mL of water and dried under vacuum overnight to yield 1.7 g (94%) of 2-ethylbenzo[g]quinoline-3-carboxylic acid as a yellow solid. FT-IR (neat): 3375.2, 3358.8, 3341.44, 3319.75, 3052.62, 2936.42, 2539.11, 2521.27, 2447.5, 2159.16, 2097.93, 2026.56, 1974.01, 1880.95, 1876.13, 1690.97, 1686.15, 1670.72, 1661.56, 1638.42, 1634.56, 1583.45, 1536.2, 1458.08, 1405.53, 1278.72, 1254.61, 1232.43, 1183.25, 1141.78, 1084.4, 1053.06, 1053.06, 966.27, 898.28, 784.97, 751.22, 686.61, 619.59 cm⁻¹. ¹H NMR (500 MHz, CD₃OD) δ: 9.89 (s, 1H), 9.13 (s, 1H), 8.83 (s, 1H), 8.32 (dd, J=11.0, 8.7 Hz, 2H), 7.85 (dddd, J=9.5, 8.0, 6.7, 1.1 Hz, 2H), 3.72 (q, J=7.6 Hz, 2H), 1.55 (t, J=7.6 Hz, 3H). ¹³C NMR (126 MHz, CD₃OD) δ: 167.20, 165.48, 152.46, 138.34, 134.92, 134.57, 133.00, 132.10, 130.46, 129.78, 129.44, 125.22, 124.31, 118.99, 28.97, and 14.45. HRMS m/z calculated for C₁₆K₄NO₂ [M+H]⁺: 252.1019; found: 252.1012.

### Example 2

### Synthesis of Monomer (3) and Dimers (4)-(11)

Compounds 3-9 (FIG. 1) were synthesized on solid phase by analogy to methods previously reported (PCT Publ. No. WO 2009/015384; Palde et al., “Strategies for Recognition of Stem-loop RNA Structures by Synthetic Ligands: Application to the HIV-1 Frameshift Stimulatory Sequence,” J. Med. Chem. 53:6018-6027 (2010), which is hereby incorporated by reference in its entirety). For compounds 10 and 11 (FIG. 1), L-pentenyl glycine was synthesized via asymmetric alkylation of pseudoephedrine glycinamide (Myers et al., “Highly Practical Methodology for the Synthesis of d- and l-α-Amino Acids, N-Protected α-Amino Acids, and N-Methyl-α-amino Acids,” J. Am. Chem. Soc. 119:656-673 (1997), which is hereby incorporated by reference in its entirety).

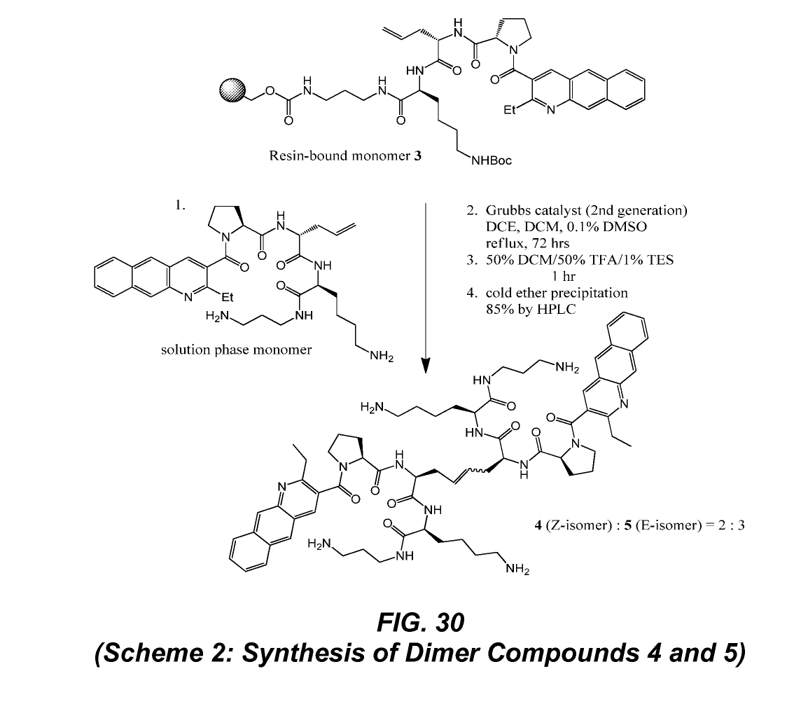

For compounds 3, 4 and 5, replacement of the disulfide in lead compound 1 with a non-labile olefin (C═C) bioisotere was performed according to procedures similar to those described in a recent report (Palde et al., “Strategies for Recognition of Stem-loop RNA Structures by Synthetic Ligands: Application to the HIV-1 Frameshift Stimulatory Sequence,”J. Med. Chem. 53:6018-6027 (2010), which is hereby incorporated by reference in its entirety). Briefly, resin bound monomer 3 (Scheme 2, FIG. 30) was synthesized using standard Fmoc methodology for solid phase peptide synthesis (“SPPS”). Wang resin (1.0 g, 100-200 n mesh) was activated with 1,1′-carbonyldiimidazole (DIC, 1.62 g, 10 mmol) in 12 mL of DMF for 12 h on a LabQuake rotator. The resin was then washed three times each with DMF, CH₂Cl₂ and again with DMF, followed by reaction with 1,3-diaminopropane (0.83 mL, 10 mmol) in DMF for another 12 h. After repeating the wash cycle, the first amino acid (Fmoc-lys(boc)-OH, 3 mmol) was coupled to the resin using HBTU (1.14 g, 3 mmol) and DIPEA (0.85 mL, 5 mmol) in 12 mL DMF and rotating the mixture for 2 h. Following the wash cycle, Fmoc deprotection was accomplished using 12 mL of 20% piperidine in DMF for 1 hour, followed again by the wash cycle. The remaining amino acids (Fmoc-L-allylglycine and Fmoc-L-proline) were similarly coupled to the growing peptide on the resin. 2-ethyl benzo[g]quinoline carboxylic acid (FIG. 1, 2) (0.502 g, 2 mmol) was coupled to the rest of the peptide using same SPPS conditions to synthesize resin-bound monomeric compound 3 (Scheme 2, FIG. 30).

Next, the resin was split into two equal parts of 0.50 g. One part was cleaved with 50% TFA/1% TES in 10 mL of CH₂Cl₂ for 1 h to obtain 3 (Scheme 2) as a yellow solid material (0.20 g) after removal of solvent. This cleaved product was used without purification as the solution component for the metathesis reaction. Both resin-bound and cleaved 3 (Scheme 2) were dried under vacuum overnight. The resin was allowed to swell in 10 mL dry CH₂Cl₂ for 10 min, washed (3×10 mL) with CH₂Cl₂, and then subjected to three 10 min washes with 0.8 M LiCl in DMF. Finally, the resin was washed (2×10 mL) with dry, degassed 1,2-dichloroethane and suspended in 5 mL of the same solvent in a 25 mL two-neck round bottom flask equipped with a reflux condenser and an N₂ inlet. The cleaved monomer 3 (Scheme 2) (0.20 g, 0.32 mmol) dissolved in 10 mL of a 1:4 mixture of CH₂Cl₂ and 1,2-dichloroethane (with two drops of DMSO to increase solubility) was added to the resin in the flask. The flask was maintained under a constant positive pressure of N₂ gas and a solution of Grubbs' second-generation metathesis catalyst (0.04 g, 0.05 mmol) dissolved in 1,2-dichloromethane (2 mL) was added. The reaction mixture was refluxed for 24 h, after which the catalyst was replenished with another 0.04 g portion and refluxed again for another 24 h. After repeating this cycle a second time, the reaction was cooled to room temperature and transferred into a standard solid-phase reaction vessel with filtering. The resin was washed with CH₂Cl₂ (3×10 mL), methanol (1×15 mL), and DMF (3×10 mL) and then suspended in 10 mL of DMF with 0.2 mL DMSO and rotated for 12 hours. Finally, the resin was washed with CH₂Cl₂ (3×10 mL) and subjected to a cleavage cocktail of 50% TFA/50% CH₂Cl₂/1% TES for 1 h to obtain crude mixture 4 and 5 (Scheme 2, FIG. 30) in a 3:2 isomer ratio.

The isomers were separated using preparative reversed-phase-HPLC on a C18 column (Waters, XBridge™ Prep C18 5 μm OBD™, 19×250 mm) using a water-acetonitrile gradient with 0.1% TFA. While E and Z olefin geometries cannot be assigned definitively in the absent an X-ray crystal structure, assignments have been proposed based on three lines of evidence: chemical shifts of the olefin protons in the ¹H NMR spectra, analysis of the IR spectra, and the known selectivity of olefin cross-metathesis reactions. In ¹H NMR, the chemical shift of the olefin proton in the E-isomer is further downfield compared to the Z-isomer (Knothe et al., “Fatty Alcohols through Hydroxylation of Symmetrical Alkenes with Welenium Dioxide/tert.-butylhydroperoxide,” J. Am. Oil Chem. Soc 72:1021-1026 (1995); Kremminger, “Asymmetric Synthesis of Unsaturated and Bis-hydroxylated (S,S)-2,7-Diaminosuberic Acid Cerivatives,” Tetrahedron 53(20):6925-6936 (1997), which are hereby incorporated by reference in their entirety). The infrared C═C stretch for the E-isomer shows absorbance at a higher frequency (1665 cm⁻¹) compared to the Z-isomer C═C stretch (1662 cm⁻¹). Also, the weak absorption at 971 cm⁻¹ for the E-isomer is consistent with spectra for trans 1,2-disubstituted alkenes (McNaughton et al., “Self-Selection in Olefin Cross Metathesis The Effect of Remote Functionality” Org. Lett. 7:733-736 (2005); Silverstein, Spectrometric Identification of Organic Compounds, Seventh Edition: pp. 85-120 (Wiley, 2005), which are hereby incorporated by reference in their entirety). The 3:2 isomer ratio is not surprising, since although the trans isomer is thermodynamically more favored in olefin metathesis reactions, this often provides only modest selectivity (Silverstein, Spectrometric Identification of Organic Compounds, Seventh Edition: pp. 85-120 (Wiley, 2005), which is hereby incorporated by reference in its entirety).

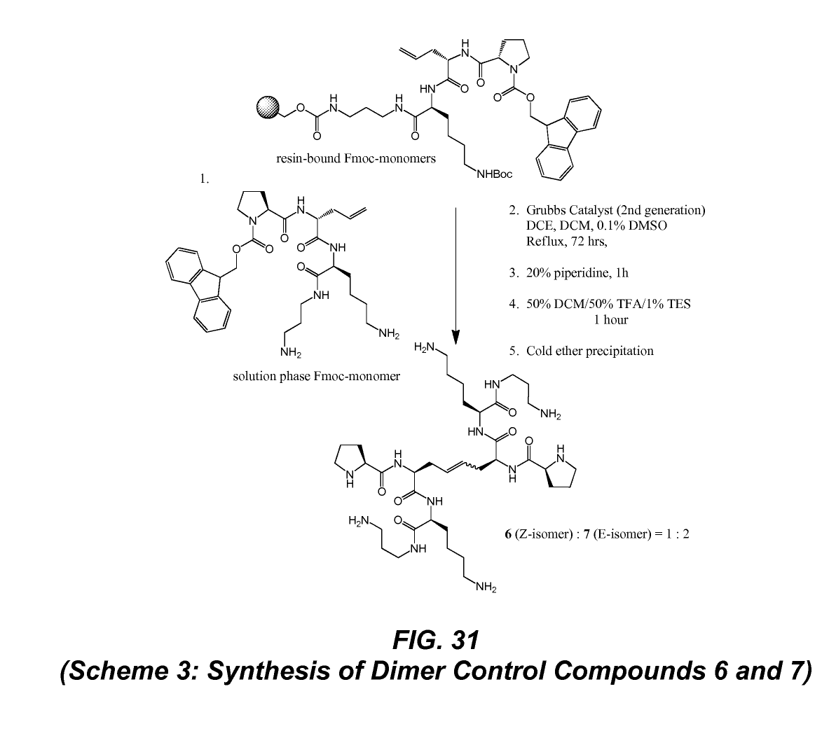

Control compounds 6 and 7 were synthesized using the SPPS procedure described above to assemble the resin-bound monomer shown in Scheme 3 (FIG. 31). However, the ethyl benzo[g]quinoline carboxylic acid heterocycle coupling step was eliminated. The resin-bound Fmoc protected monomer was then subjected to the olefin metathesis reaction in the presence of the Fmoc-monomer solution component as describe supra. After the reaction was completed, the Fmoc group was removed with 20% piperidine in DMF before the peptidic product was cleaved from the resin with 50% TFA/50% CH₂Cl₂. The solvent was removed and the product was precipitate twice from cold diethyl ether. The crude mixture of compounds 6 and 7 (Scheme 3, FIG. 31) was purified and separated by reverse-phase preparative HPLC to obtain compounds 6 and 7 (Scheme 3, FIG. 31) in a Z:E geometrical isomer ratio of 1:2. The products were confirmed by mass spectrometry and olefin geometries were assigned as described above.

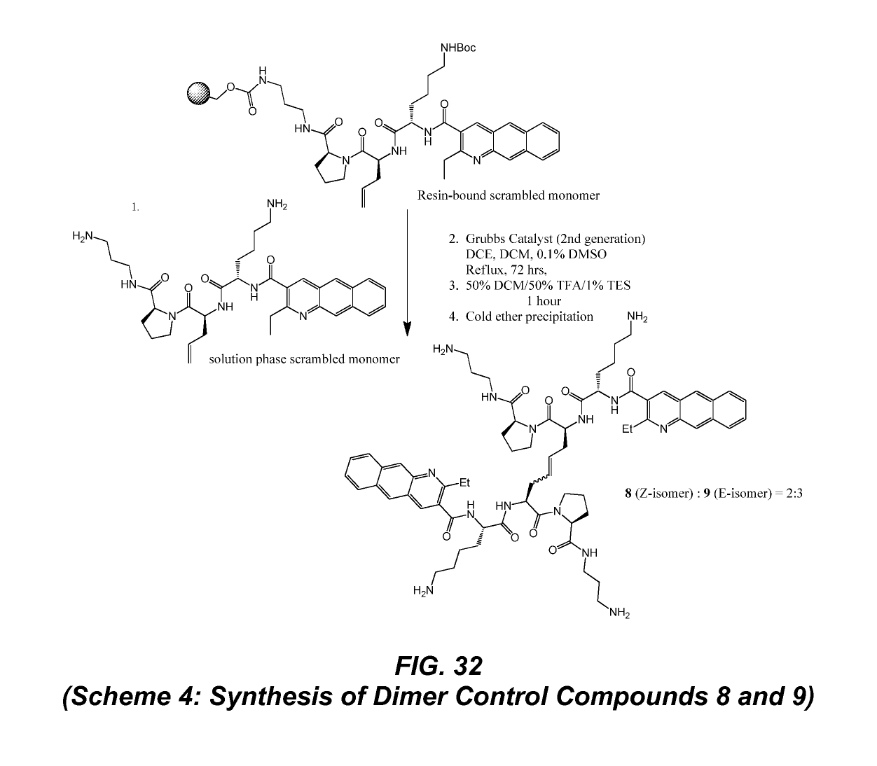

Control compounds 8 and 9 (Scheme 4, FIG. 32) are constitutional isomers of compounds 4 and 5 (Scheme 2, FIG. 30) in which the amino acid sequence Lys-Algly-Pro-Benzo[g]quinoline was re-ordered as Pro-Algly-Lys-Benzo[g]quinoline. This was done to confirm that binding to a specific RNA sequence depends both on the amino acids incorporated into the peptide and their relative order. The peptide coupling procedure describe above was used to synthesize the resin bound scrambled monomer. The resin-bound scrambled monomer was split into two equal parts of 0.5 g each. One part was cleaved and used as solution phase component for olefin metathesis reaction employing Grubbs' second-generation catalyst (Scheme 4, FIG. 32). The products were isolated and purified by reversed phase preparative HPLC, to yield compounds 8 and 9 (Scheme 4, FIG. 32) as a 2:3 ratio of Z- and E isomers respectively. Olefin geometries were assigned as described above.

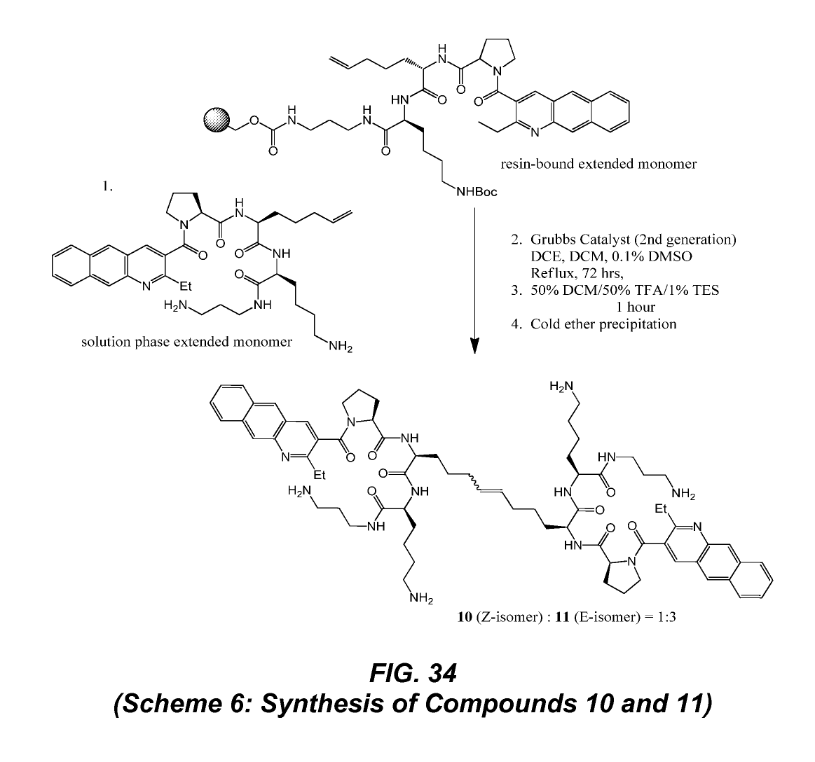

Compounds 10 and 11 (Scheme 6, FIG. 34) were synthesized by slight modification to the procedures for the synthesis of compounds 4-9. To obtain the required 8-carbon linker spacing, the (L)-Fmoc-Allylglycine was substituted with the unnatural amino acid (L)-Fmoc-pentenylglycine which served as the olefin for the dimerization of the monomer by self metathesis reaction employing Grubbs' second generation catalyst.

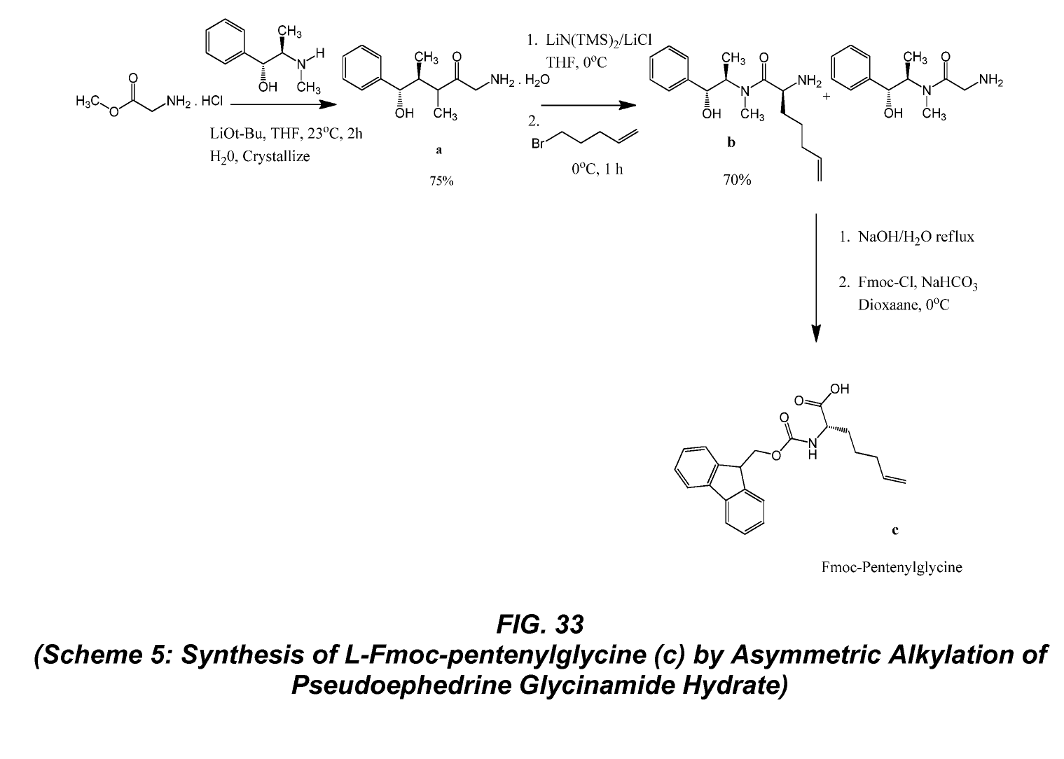

L-N-Fmoc-pentenylglycine was synthesized using a procedure reported by Myers et al., “Highly Practical Methodology for the Synthesis of d- and l-α-Amino Acids, N-Protected α-Amino Acids, and N-Methyl-α-amino Acids,” J. Am. Chem. Soc. 119:656-673 (1997), which is hereby incorporated by reference in its entirety, in which (R,R)-(−)-pseudoephedrine is used as a chiral auxiliary for asymmetric alkylation. This is illustrated in Scheme 5 (FIG. 33). Briefly, (R,R)-(−)-pseudoephedrine was converted to pseudoephedrine glycinamide hydrate (Scheme 5, a) by reaction with glycine methyl ester hydrochloride using methods described in the literature.

(S)-2-amino-N-((1R,2R)-1-hydroxy-1-phenylpropan-2-yl)-N-methylhept-6-enamide (Scheme 5, b): Anhydrous lithium chloride (5.29 g, 125.2 mmol, 4.00 equiv) was taken in an oven-dried, 3-necked round-bottom flask equipped with two glass stoppers and an inlet adapter connected to a source of vacuum. Vacuum was applied to the flask and the solid lithium chloride was dried using a low flame. The flask was then cooled to 23° C. and flushed with nitrogen.

Tetrahydrofuran (90 mL) was added to the flask through a pressure equalizing addition funnel and the resulting suspension was stirred at 23° C. for 20 minutes. (R,R)-(−)-pseudoephedrine glycinamide hydrate (Scheme 5, a) (7.5 g, 31.3 mmol, 1 equiv) was added to the reaction mixture in portions for 5 minutes resulting in a cloudy solution. The solution was cooled to 0° C. in an ice bath and a 1 M solution of lithium hexamethyldisilazide in tetrahydrofuran (100 mL, 100 mmol, 3.20 equiv) was added dropwise via addition funnel. The speed of the addition was regulated such that the temperature of the reaction did not exceed 3° C. as monitored by a thermometer inserted in the reaction flask via an adapter. After the addition of base was complete, the reaction mixture was stirred at 0° C. for 20 minutes, and 5-bromopentene (4.9 g, 32.8 mmol, 1.05 equiv) was added slowly by syringe. The reaction mixture was stirred at 0° C. for 1 h. Water (75 mL) was added and the resulting biphasic mixture was acidified to pH 0 by the addition of aqueous hydrochloric acid (6 M, 45 mL). The acidified aqueous solution was then extracted with ethyl acetate (100 mL). The ethyl acetate layer was separated and extracted sequentially with single 50 mL portions of 3 M and 1 M aqueous hydrochloric acid solution, respectively. The aqueous layers were combined and cooled to 5° C. by stirring in an ice-water bath. The cold solution was basified to pH 14 by the addition of 50% aqueous sodium hydroxide solution (30 mL). The basified solution was then extracted sequentially with one 120-mL portion and three 40-mL portions of dichloromethane. The combined organic extracts were dried over anhydrous solid potassium carbonate, filtered and concentrated in vacuo resulting in a yellow oily product. Attempts to recrystallize the crude product by analogy to Myers et al., “Highly Practical Methodology for the Synthesis of d- and l-α-Amino Acids, N-Protected α-Amino Acids, and N-Methyl-α-amino Acids,” J. Am. Chem. Soc. 119:656-673 (1997), which is hereby incorporated by reference in its entirety, were not successful. However, unreacted £R, R)-(−)-pseudoephedrine glycinamide hydrate did crystallize, allowing its separation from the desired (S)-2-amino-N-((1R,2R)-1-hydroxy-1-phenylpropan-2-yl)-N-methylhept-6-enamide (Scheme 5, b) (70%, HPLC). This crude product was used for the next stage of the reaction without further purification.

Conversion of (Scheme 5, b) to Fmoc-L-pentenylglycine: To impure (Scheme 5, b) (8.5 g, 29 3 mmol, 1 equiv) in a 100 mL round bottom flask was added aqueous 1 M sodium hydroxide (58.6 mmol, 2 equiv.) and water (40 mL). The resulting solution was heated to reflux for 2 h and then allowed to cool to room temperature. Pseudoephedrine was observed to crystallize from the solution upon cooling. The solids were filtered via vacuum filtration and the aqueous solution was extracted with dichloromethane (50 mL and 30 mL) to remove residual pseudoephedrine. The organic layers were individually back-extracted with water (30 mL); the aqueous extracts were then combined with the original solution and concentrated in vacuo to a volume of 45 mL. To this concentrated solution dioxane (45 mL) and sodium bicarbonate (4.92 g, 58.6 mmol, 2 eq) were added, followed by cooling in an ice bath for 20 minutes. 9-flourenylmethoxychloride (8.3 g, 32.2 mmol, 1.1 eq) was added to the cooled solution and the reaction stirred for 3 h (the first 1 h in an ice bath). Water (250 mL) was added, and the solution was washed with 1:1 ethyl acetate:ether (400 mL). The organic layer was washed with a 2% sodium bicarbonate solution (100 mL). The aqueous layers were combined and acidified to pH 1 with 1 N HCl and then extracted with ethyl acetate (2×100 mL). The organic layers were dried over sodium sulfate and concentrated in vacuo. The residue was dissolved in toluene (20 mL) and concentrated in vacuo to remove residual dioxane. The residue was again dissolved in chloroform (2×20 mL) and concentrated in vacuo yielding an amorphous solid (6.9 g, 67% yield) of impure Fmoc-pentenylglycine. Part of this impure product was purified by preparative HPLC for spectral characterization purposes, and the other part was used in the synthesis of compounds 10 and 11 (Scheme 6, FIG. 34) without purification.

First the Fmoc-pentenyglycine was incorporated into the synthesis of the extended monomer, which was subjected to olefin self-metathesis in the presence of solution phase extended monomer. The HPLC trace for the monomer showed two major peaks: one resulting from Fmoc-pentenylglycine and the other from a non-alkylated glycine impurity. The final crude reaction product showed the disappearance of the extended monomer, as well as the formation of compounds 10 and 11 (Scheme 6, FIG. 34) as a mixture of isomers. These were separated by prep-HPLC and assigned as described above.

### Example 3

### Analysis of Dimer Binding Affinity via Surface Plasmon Resonance

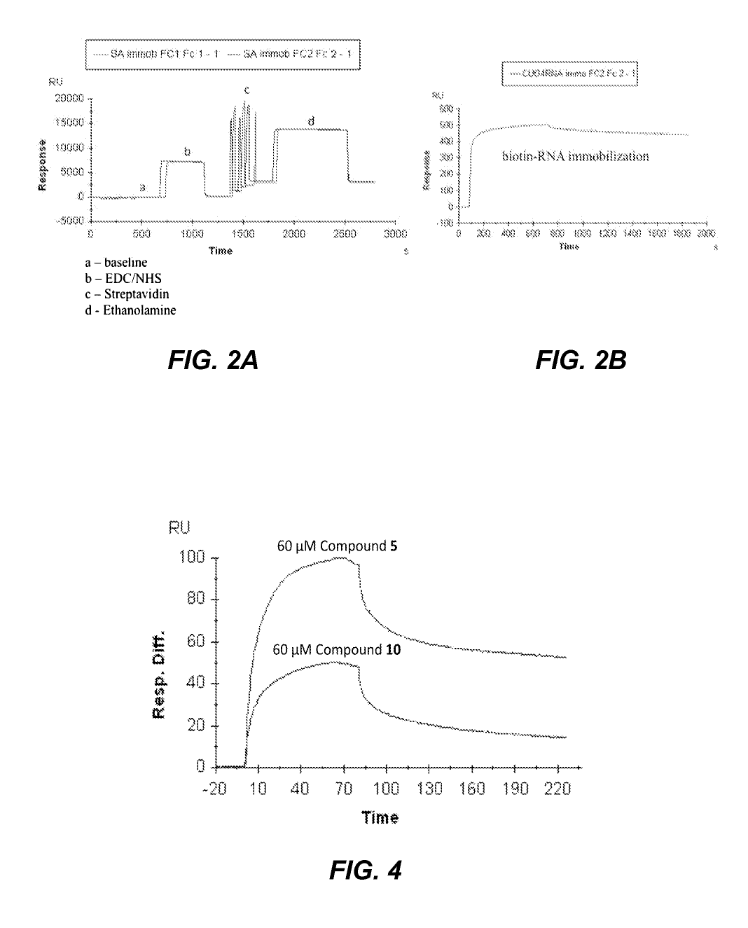

SPR binding measurements were performed on a Biacore-X instrument (Biacore, Inc., Uppsala, Sweden) with two flow channels (FC1 and FC2). 5′-Biotinylated-RNA sequences, with a C₆ linker separating the biotin label from the RNA (Integrated DNA Technologies Inc.) were immobilized on streptavidin (Rockland Immunochemicals) functionalized carboxylmethyl dextran coated sensor chips (CM5, G.E. Healthcare) using EDC/NHS (Advanced ChemTech) coupling chemistry. Filtered (0.2μ), degassed and autoclaved HBS-N buffer (0.01M Hepes, pH=7.4, 0.15 M NaCl) was employed as sample and as running buffer for all SPR experiments. A typical protocol for an experiment is as follows: A CM5 sensor chip was allowed to equilibrate to room temperature and then docked into the instrument. Following priming with running buffer, FC1 and FC2 were conditioned by manual injection of 20 μL aqueous NaOH (50 mM) at a flow rate of 30 μL/min. This was repeated 3 times followed by a wash command. Next, the carboxyl groups on the sensor chip surface in both flow cells were activated separately by injecting 60 μL of freshly prepared 1:1 mixture of EDC (0.4 M) and NHS (0.1 M) at a 5 μL/min flow rate followed by a wash. Streptavidin (100 μg/mL in 10 mM sodium acetate buffer, pH=5.0) was immediately injected in pulses until approximately between 2500-3000 RU was achieved in both flow cells. After washing, the surface was deactivated with 60 μL injection of ethanolamine (1M, pH=8.5) at 5 μL/min. The flow cells were primed, and the streptavidin surface in FC1 alone was blocked with 30 μL of biotin (20 μM in HBS-N) at 5 μL/min. The RNA (200 nM in running buffer, HBS-N) to be immobilized was unfolded by heating above its predicted melting temperature in a heated block for 2 min and then allowed to refold by cooling gradually to room temperature. The RNA was then immobilized in FC2 alone to response units ranging from 200-1000 RU using a 5 μL/min flow rate. Representative sensorgrams for the immobilization procedure is shown in FIGS. 2A-B. Two 20 μL aliquots of NaCl (0.5 M) were injected at a 30 μL/min flow rate to remove non-specifically bound RNA. The level of RNA immobilized was noted when the baseline was stable, usually after repeated buffer injection followed by a “prime” command. Binding measurements were performed by flowing various concentrations of the compounds to be analyzed over the immobilized RNA and recording the reference-subtracted (FC2−FC1) sensorgrams. For kinetic analysis, at least five sensorgrams corresponding to different concentrations of compound (usually ranging from 0.05-1 μM in HBS-N) were obtained using flow rates of either 60 μL/min or 30 μL/min. The experimental sensorgrams were globally fitted to a 1:1 binding equation (Biaevaluation software) to obtain association rates (kₐ) and dissociation rates (kₐ) as well as equilibrium binding constants (K_{D}). To measure the binding stoichiometry (n), the relations in equations (1-3) below were used:

$$\begin{matrix}{RU}_{max} = \frac{{MW}_{compound}}{{MW}_{RNA}} \times {RU}_{RNA} \times Stocichometry ( n ) & ( 1 ) \\ {RU}_{( max ) predict} = n \times {RU}_{( max ) observed} & ( 2 ) \\ r = \frac{{RU}_{eq}}{{RU}_{max}} & ( 3 )\end{matrix}$$

where RUₘₐₓ is the maximum resonance response unit at saturation, RU_{RNA} is the amount RU of RNA immobilized, n is the stoichiometry, MW_{compound} and MW_{RNA} are molecular weights of compound and RNA respectively, and RU_{eq} represents the resonance response at steady-state (equilibrium). The response units at equilibrium were subjected to Scatchard binding plots of (r/C_{free} vs. r).

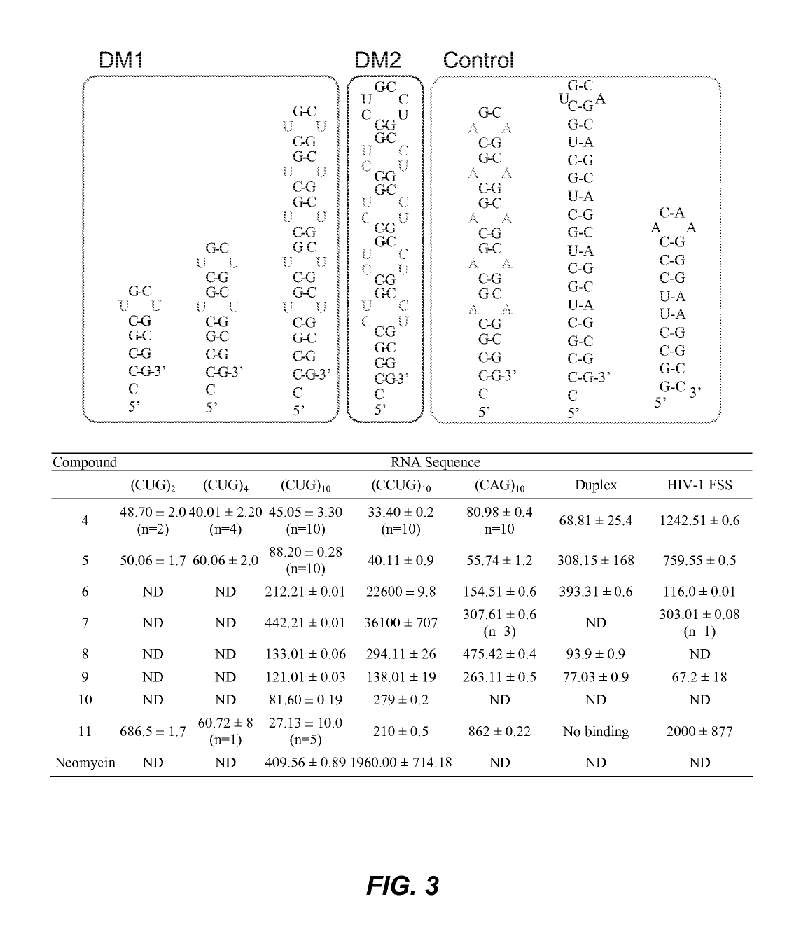

The binding characteristics of compounds 3-11 were evaluated against pathogenic CUG^{exp} RNA and other RNA sequences by SPR. This technique allows the association and dissociation rates (kₐ and k_{d}), equilibrium binding constants (K_{D}), and the binding stoichiometry (n) to be measured in a label-free format (Liu and Wilson, “Quantitative Analysis of Small Molecule-nucleic Acid Interactions with a Biosensor Surface and Surface Plasmon Resonance Detection,” Methods Mol. Biol. 613:1-23 (2010), which is hereby incorporated by reference in its entirety). RNA sequences, shown in FIG. 3 along with their respective abbreviated names, were designed to test the effect of increasing numbers of CUG repeats (“(CUG)₂” (SEQ ID NO: 1), “(CUG)₄” (SEQ ID NO: 2), and “(CUG)₁₀” (SEQ ID NO: 3)), differences between recognition of DM1 and DM2 RNA (“(CCUG)₁₀” (SEQ ID NO: 4)), U-U vs. A-A mismatches (“(CAG)₁₀” (SEQ ID NO: 5)), and mismatched RNA vs. a CUG-CAG duplex (“Duplex”) (SEQ ID NO: 6). An RNA sequence derived from the gag/pol frameshift-stimulating region of HIV-1 (“HIV-1 FSS”) (SEQ ID NO: 7) (Staple and Butcher, “Solution Structure of the HIV-1 Frameshift Inducing Stem-loop RNA,” Nucl. Acids Res. 31, 4326-4331 (2003), which is hereby incorporated by reference in its entirety) was employed as an off-target control.

Compound 3 showed no measurable affinity for (CUG)₁₀. In contrast, compounds 4 and 5 bound (CUG)₁₀ with apparent K_{D} values of 45.05±3.30 nM and 88.20±0.28 nM, respectively, in both cases representing a roughly 50-fold improvement in affinity relative to compound 1 (6.7±0.2 μM, as measured by filter binding (Gareiss et al., “Dynamic Combinatorial Selection of Molecules Capable of Inhibiting the (CUG) Repeat RNA—MBNL1 Interaction In Vitro: Discovery of Lead Compounds Targeting Myotonic Dystrophy (DM1),” J. Am. Chem. Soc. 130:16254-16261 (2008), which is hereby incorporated by reference in its entirety)). Scatchard analysis of the steady-state SPR response units (RU) for the binding between compounds 4 or 5 and (CUG)₁₀ yielded a binding stoichiometry of 10:1 for each isomer. This implies that one molecule of compound binds one (CUG) trinucleotide in the (CUG)₁₀ RNA hairpin stem and loop. The Scatchard plot for these data displays a strong convex curvature (Table 3), and Hill coefficients of 1.9 (compound 4) or 1.5 (compound 5) were calculated, all consistent with positively cooperative binding (Munde et al., “DNA Minor Groove Induced Dimerization of Heterocyclic Cations: Compound Structure, Binding Affinity, and Specificity for a TTAA Site,” J. Mol. Biol. 402:847-864 (2010); Byers, “Probe-dependent Cooperativity Patterns in Hill-plots,” J. Chem. Educ. 54:352 (1977), which are hereby incorporated by reference in their entirety). Both the apparent affinity and binding stoichiometry (the latter as a function of number of CUG repeats) remained consistent for compound 4 binding to (CUG)₄ and (CUG)₂, confirming that one molecule of compound 4 binds to each CUG. In contrast, the affinity of compound 5 actually appeared to strengthen slightly for (CUG)₄ and (CUG)₂, although the amount is not likely to be significant (FIG. 3). Reducing the number of CUG repeats in the target RNA caused the curvature in Scatchard plots for compound 4 to become less convex, approaching linearity at a repeat length of 2. This indicates loss of cooperativity as the number of CUG repeats decreases. Likewise, calculated Hill coefficients were consistent with noncooperative binding to short repeats. One possible explanation for this is that the longer (CUG)₁₀ repeat RNA has greater flexibility, and ligand binding induces a conformational change which then affects the mode of binding of subsequent ligand molecules. While compounds 4 and 5 are strongly selective for binding CUG repeat RNAs relative to unrelated hairpins such as the HIV-1 FSS (apparent K_{D} 1242.51±0.6 nM and 759.55±0.5 nM, respectively), both (CCUG)₁₀ and (CAG)₁₀ were bound with similar affinities to the CUG repeat sequences. It is not surprising that differentiation among these sequences is particularly challenging. MBNL1 itself has been reported to bind CUG and CCUG repeats with only a two-fold difference in affinity as measured by gel-shift assay (Warf et al., “MBNL Binds Similar RNA Structures in the CUG Repeats of Myotonic Dystrophy and its Pre-mRNA Substrate Cardiac Troponin T,” RNA 13:2238-2251 (2007), which is hereby incorporated by reference in its entirety). Likewise, MBNL1 was found to bind (CUG) and (CAG) repeats with similar affinity in a filter binding assay (Yuan et al., “Muscleblind-like 1 Interacts with RNA Hairpins in Splicing Target and Pathogenic RNAs,” Nucl. Acids Res. 35:5474-5486 (2007), which is hereby incorporated by reference in its entirety). The X-ray crystal structures of a (CUG)₆ repeat duplex and a (CAG) repeat containing RNA duplex showed that both adopt A-form RNA conformation with alternating stripes of positive and negative potential due to the G-C pairs (Mooers et al., “The Structural Basis of Myotonic Dystrophy from the Crystal Structure of CUG Repeats,” Proc. Natl. Acad. Sci. U.S.A. 102:16626-16631 (2005); Kiliszek et al., “Atomic Resolution Structure of CAG RNA Repeats: Structural Insights and Implications for the Trinucleotide Repeat Expansion Diseases,” Nucl. Acids Res. 38:8370-8376 (2010); Kiliszek et al., “Structural Insights into CUG Repeats Containing the ‘Stretched U-U Wobble’: Implications for Myotonic Dystrophy,” Nucl. Acids Res. 37:4149-4156 (2009), which are hereby incorporated by reference in their entirety). It has been predicted that the (CCUG) repeat RNA may also adopt a similar conformation (Kiliszek et al., “Structural Insights into CUG Repeats Containing the ‘Stretched U-U Wobble’: Implications for Myotonic Dystrophy,” Nucl. Acids Res. 37:4149-4156 (2009), which is hereby incorporated by reference in its entirety). Binding of compound 4 to duplex CUG-CAG appears to be approximately 5.7-fold stronger than the E isomer, compound 5.

To ascertain the importance of the benzo[g]quinoline moiety, cis (6) and trans (7) peptides lacking the heterocycle were examined. While both compounds bound (CUG)₁₀ RNA with modest affinity, neither displayed any selectivity. Likewise, benzo[g]quinoline-bearing compounds with a “scrambled” peptide sequence (compounds 8 and 9) showed reduced affinity to (CUG)₁₀, and no selectivity for CUG or CCUG repeats relative to duplex CUG-CAG or HIV-1 FSS RNAs. Both observations indicate that a substantial fraction of the functionality in compounds 4 and 5 must be present in the correct relative spatial orientation in order for high affinity recognition to be achieved.

A key aspect of the original RBDCC library design was that the disulfide was incorporated only to reversibly link RNA-binding modules together, rather than participating in RNA binding itself. Such a reversible linkage is a useful feature of any dynamic combinatorial library, but depending on the exchange reaction used may require subsequent re-engineering of the compound in a form not subject to exchange under physiological conditions. In this case, replacement of the labile disulfide bridge with an olefin permitted not only improvement of the biostability of the compound, but also testing of the effect of varying the spacing between modules on binding. This was also motivated by the complete lack of binding by monomer 3 and benzo[g]quinoline 2, indicating that a bidentate mode is needed to stabilize binding by the two heterocyclic handles. If this hypothesis holds, then changing the separation between the two halves of the ligand should alter selectivity and affinity. Thus, “extended” linker compounds 10 and 11 were synthesized. These compounds were found to bind (CUG)₁₀ with an affinity similar to that displayed by compounds 4 and 5. However, affinity decreased as hairpin length decreased (from an apparent K_{D} of 27.93±10 nM for (CUG)₁₀ to 686.5±1.7 nM for (CUG)₂). Binding stoichiometry also changed, with 5:1 binding observed for (CUG)₁₀ and 1:1 binding observed for (CUG)₄. This more stringent structural requirement also manifested as a dramatic increase in sequence selectivity: Compound 11 has a significant preference for (CUG)₁₀ over (CCUG)₁₀, and a 32-fold preference vs. (CAG)₁₀. No measurable binding was observed by SPR to the duplex CUG-CAG sequence, while binding to the HIV hairpin was reduced 75-fold. As compound 10 was isolated as only a very minor product of the metathesis reaction, only a limited number of experiments were possible with this compound. However, SPR data indicates it is also somewhat selective for (CUG)₁₀ over (CCUG)₁₀. Likewise, compound 10 binds (CUG)₁₀ with a 5:1 stoichiometry. Binding of compound 11 to (CUG)₁₀ is cooperative (Hill coefficient of 1.9). The two-fold difference in stoichiometry for “extended” compounds 10 and 11 relative to compound 4 and 5 is readily observable in the SPR trace. For example, an injection of excess compound 10 produces roughly half the steady-state response of the injection of an equivalent concentration of compound 5 (see FIG. 4). These data are consistent with the requirement of a more distributed binding site. Neomycin, a well-studied aminoglycoside antibiotic with relatively low sequence selectivity, has been reported to bind (CUG) repeats (Warf et al., “Pentamidine Reverses the Splicing Defects Associated with Myotonic Dystrophy,” Proc. Natl. Acad. Sci. U.S.A. 106:18551-18556 (2009), which is hereby incorporated by reference in its entirety), and represents a useful positive control from another structural class. It was found that neomycin binds (CUG)₁₀ and (CCUG)₁₀ with much weaker affinity (apparent K_{D} of 409.56±0.89 and 1960.00±714.18 nM, respectively) than the best compounds described above.

For SPR-derived kinetic constants, it was observed that on-rates generally do not vary substantially for individual compounds across a series of RNAs. In contrast, off rates strongly reflect differences in selectivity, consistent with the above hypothesis (Table 3 and Table 4).

TABLE 3
On-rates (kₒₙ, M⁻¹s⁻¹)

| Compound | (CUG)₂ | (CUG)₄ | (CUG)₁₀ | (CCUG)₁₀ | (CAG)₁₀ | Duplex | HIV-1 FSS |
|---|---|---|---|---|---|---|---|
| 4 | 2.45E+04 | 4.24E+04 | 2.78E+04 | 6.01E+04 | 3.56E+04 | 2.95E+04 | 1.13E+04 |
| 5 | 2.01E+04 | 3.01E+04 | 2.84E+04 | 4.97E+04 | 5.02E+04 | 1.50E+04 | 5.19E+04 |
| 6 | | | 1.32E+05 | 1.36E+04 | 1.25E+05 | | 2.21E+05 |
| 7 | | | 1.08E+05 | 1.31E+04 | 1.08E+05 | 1.68E+05 | 6.56E+04 |
| 8 | | | 2.46E+04 | 1.54E+04 | 1.26E+04 | 2.90E+04 | |
| 9 | | | 2.42E+04 | 1.99E+04 | 1.57E+04 | 2.43E+04 | 3.23E+04 |
| 10 | | | | 1.92E+04 | | | |
| 11 | 9.93E+04 | 2.51E+04 | 3.18E+04 | 9.10E+04 | 1.42E+04 | No Binding | 1.47E+04 |
| Neomycin | | | 1.25E+05 | 1.09E+05 | | | |

TABLE 4
Off-rates (k_{off}, s⁻¹)

| Compound | (CUG)₂ | (CUG)₄ | (CUG)₁₀ | (CCUG)₁₀ | (CAG)₁₀ | Duplex | HIV-1 FSS |
|---|---|---|---|---|---|---|---|
| 4 | 8.83E−04 | 1.63E−03 | 1.18E−03 | 1.84E−03 | 2.89E−03 | 1.53E−03 | 1.53E−02 |
| 5 | 7.69E−04 | 1.39E−03 | 2.47E−03 | 9.87E−04 | 2.73E−03 | 3.84E−03 | 3.93E−03 |
| 6 | | | 3.48E−02 | 2.99E−01 | 1.92E−02 | | 2.57E−02 |
| 7 | | | 4.58E−02 | 4.72E−01 | 4.58E−02 | 5.67E−02 | 1.98E−02 |
| 8 | | | 3.03E−03 | 4.53E−03 | 5.93E−03 | 2.71E−03 | |
| 9 | | | 2.85E−03 | 2.71E−03 | 3.44E−03 | 1.86E−03 | 2.00E−03 |
| 10 | | | | 5.34E−03 | | | |
| 11 | 6.82E−03 | 1.52E−03 | 6.65E−04 | 2.10E−03 | 1.23E−02 | No Binding | 2.62E−02 |
| Neomycin | | | 5.07E−02 | 1.95E−01 | | | |

The data in Table 3 and Table 4 are perhaps easier to conceptualize in terms of dissociative half-life, or t_{1/2}, for the interaction with target sequences (Table 5). Comparisons among compounds and target sequences based on dissociative half-life are a useful addition to commonly employed metrics such as the selectivity index, and may have some advantages since the former is an absolute measurement while the latter is defined based on a changeable set of RNAs. For the compounds studied, compound 11 displayed the largest differences in t_{1/2}, consistent with its highest selectivity for CUG repeats: an approximately 3-fold difference between (CUG)₁₀ and (CCUG)₁₀, and an approximately 40-fold difference between (CUG)₁₀ and the HIV-1 FSS.

TABLE 5
Residence time (t_{1/2}, seconds) for compound binding to RNA

| Compound | (CUG)₂ | (CUG)₄ | (CUG)₁₀ | (CCUG)₁₀ | (CAG)₁₀ | Duplex | HIV-1 FSS |
|---|---|---|---|---|---|---|---|
| 4 | 784.82 | 425.15 | 587.29 | 376.63 | 239.79 | 452.94 | 45.29 |
| 5 | 901.17 | 498.56 | 280.57 | 702.13 | 253.85 | 180.47 | 176.34 |
| 6 | | | 19.91 | 2.32 | 36.09 | | 26.96 |
| 7 | | | 15.13 | 1.47 | 15.13 | 12.22 | 35.00 |
| 8 | | | 228.71 | 152.98 | 116.86 | 255.72 | |
| 9 | | | 243.16 | 255.72 | 201.45 | 372.58 | 346.50 |
| 10 | | | 192.5 | 129.78 | | | |
| 11 | 101.61 | 455.92 | 1042.11 | 330.00 | 56.34 | NB | 26.45 |
| Neomycin | | | 13.70 | 3.55 | | | |

“NB” = no binding.

### Example 4

### Analysis of Dimer Equilibrium Affinities via Fluorescence Titration

The benzo[g]quinoline moiety is fluorescent, with excitation and emission maxima at 362 nm and 439 nm respectively in methanol. Fluorescence titration binding measurements were performed on a Cary Eclipse fluorescence spectrophotometer using a 10 mm path-length semimicro quartz fluorescence cell with 400 μL sample holding capacity. Absorbance measurements were carried out on a UV-Visible spectrophotometer (Shimadzu, UV-1601PC) using a 1 mL (1 cm path-length) quartz cell. Spectroscopic grade anhydrous methanol used in the measurement of relative quantum yield of the 2-ethyl benzo[g]quinoline carboxylic acid was purchased from Alfa Aesar and was used without further purification. For these measurements, known procedures were used (Lee and Grissom, “Design, Synthesis, and Characterization of Fluorescent Cobalamin Analogues with High Quantum Efficiencies,” Org. Lett. 11:2499-2502 (2009); Joseph R. Lakowicz, Principles of Fluorescence Spectroscopy, 2^{nd} edition (Kluwer Academic/Plenum Publishers, 1999), which are hereby incorporated by reference in their entirety). Briefly, RNA (10 μM stock) in HBS-N buffer was titrated into a solution of 1 μM compound in HBS-N. After each addition of RNA, ten minutes were allowed for equilibration before recording the change in fluorescence at 468.5 nm. Fluorescence units (“FU”) were then corrected for dilution and the FU after each addition was subtracted from the FU at zero RNA concentration to give AFU. Following saturation, AFU was plotted against RNA concentration using Origin 7 (OriginLab, Inc.), and fitted to a 1:1 Langmuir binding model to obtain apparent dissociation constants (Kₙ). The reported K_{D} are averaged from at least two repetitions.

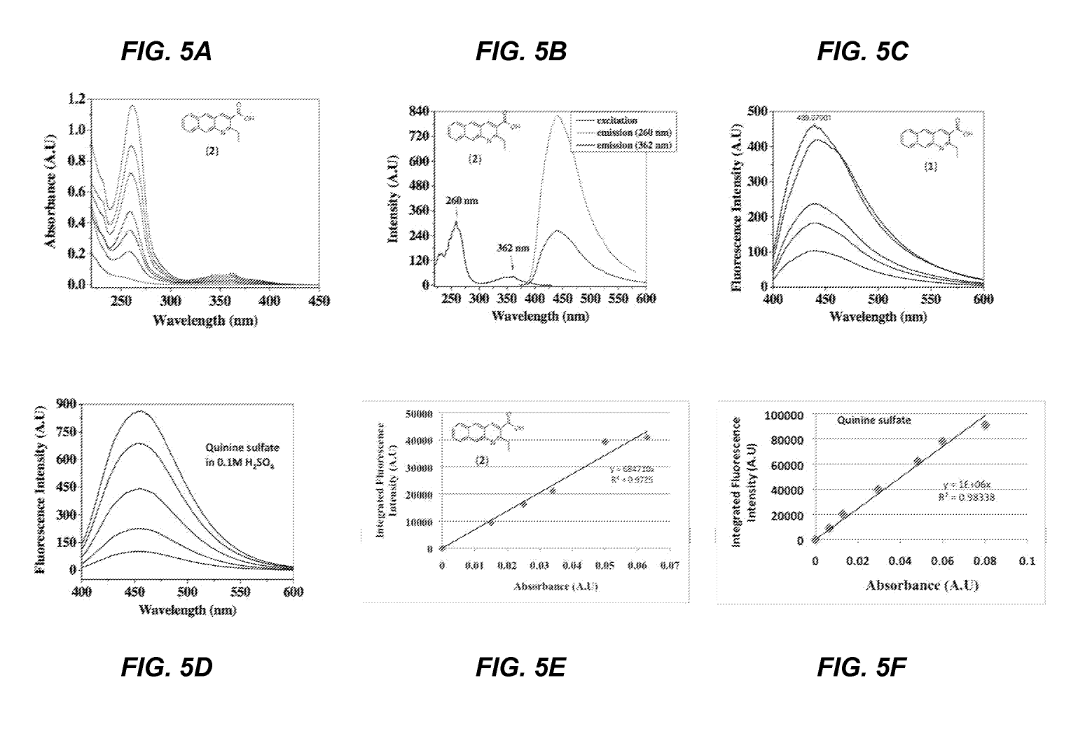

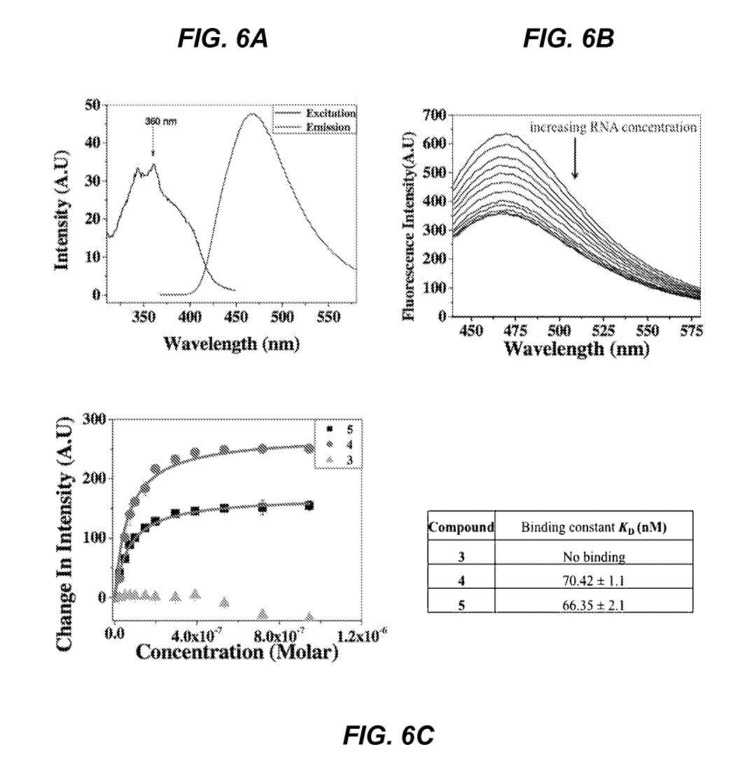

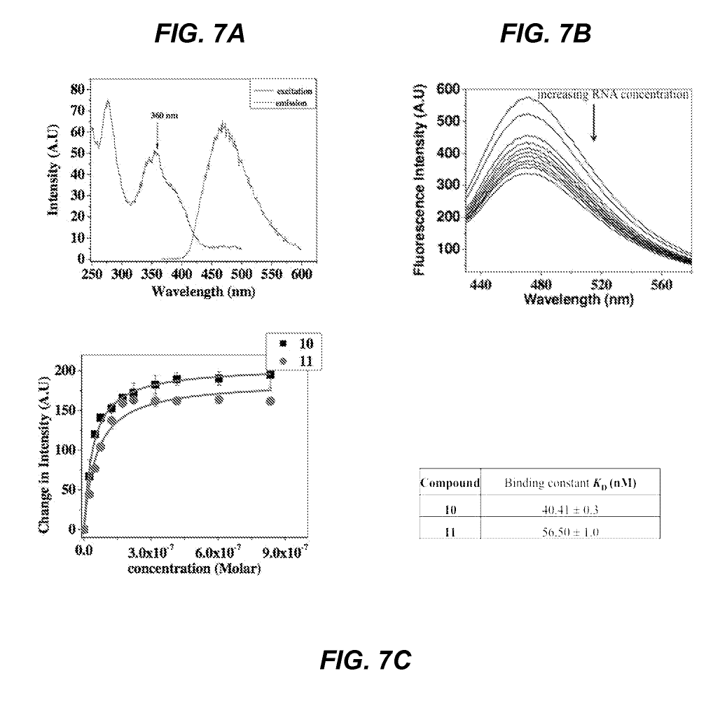

Selected equilibrium affinities for (CUG)₁₀ were confirmed by fluorescence titration. The quantum yield of the ethylbenzo[g]quinoline carboxylic acid (compound 2), relative to quinine sulfate, was determined to be 0.64 (FIGS. 5A-F). This unique feature allowed the direct monitoring of RNA binding, and mammalian cell penetration and localization without the need for additional labeling. In all cases, compound binding resulted in saturable quenching of fluorescence (FIGS. 6A-C and FIGS. 7A-C). Apparent K_{D} values measured in this manner are consistent with those obtained by SPR (Table 6); that they are not identical to SPR-measured values likely results from the differing formats of the two methods. As a control, yeast tRNA was titrated into a mixture of compounds 4 and 5. No binding was observed.

TABLE 6
Binding constants as measured by fluorescence titration

| | | Apparent |
|---|---|---|
| Compound | Sequence | K_{D} (nM) |
| 3 | (CUG)₁₀ | No binding |
| 4 | (CUG)₁₀ | 66.35 ± 2.1 |
| 5 | (CUG)₁₀ | 70.42 ± 1.1 |
| 10 | (CUG)₁₀ | 40.4 ± 0.3 |
| 11 | (CUG)₁₀ | 56.5 ± 1.0 |
| 11 | (CCUG)₁₀ | 73.6 ± 0.8 |

The titration of 11 into (CCUG)₁₀, shown in FIGS. 9A and 9B, is representative. Increasing amounts of RNA cause a concentration-dependent decrease in the fluorescence of the benzo[g]quinoline moiety, reaching saturation at a concentration consistent with SPR results. As may be observed in Table 6, the fluorescence assay does not show the same selectivity for 11 binding to (CUG) repeats relative to (CCUG) repeats that was observed in the SPR measurements; this may be due to the differing formats of the two assays.

### Example 5

### In Vitro Cellular Studies with Dimers

Prior to analyzing the ability of selected compounds to interfere with CUG^{exp} mediated effects in model cell lines, it was crucial to first establish that they are capable of crossing the cell membrane, and are non-toxic at experimentally relevant concentrations. Cell permeability was assessed in human fibroblasts and in mouse C2C12 myoblasts.

For cell permeability studies, human fibroblasts or mouse myoblasts grown to 80% confluence were exposed to compounds for 12 hours in a 96 well tissue culture plate. After removal of the culture media (DMEM, with 10% FBS, 1% pen-strep (GIBCO)) the cells were washed twice with PBS to remove excess compounds. Cells were then imaged while in buffer under a fluorescence microscope (Olympus IX70) in the 96-well plate using a 460 nm emission filter.

For toxicity studies, mouse myoblasts or human fibroblasts were plated in a 96-well tissue culture plate in DMEM (10% fetal bovine serum, 100 units/ml penicillin, 100 mg/ml streptomycin) and allowed to grow to approximately 80% confluence at 37° C. under CO₂. Varying compound concentrations (up to 1 mM) were incubated with cells (48 hours, 37° C.). Media was then removed, and 3-(4,5-dimethylthiazol-2-yl)-2,5-diphenyltetrazolium bromide (“MTT”) in media was added to each well and incubated at 37° C. for 4 h. After removal of the MTT media, isopropanol (100 pp was added, and absorbance was measured at 600 nm on a Modulus microplate reader (Turner Biosystems).

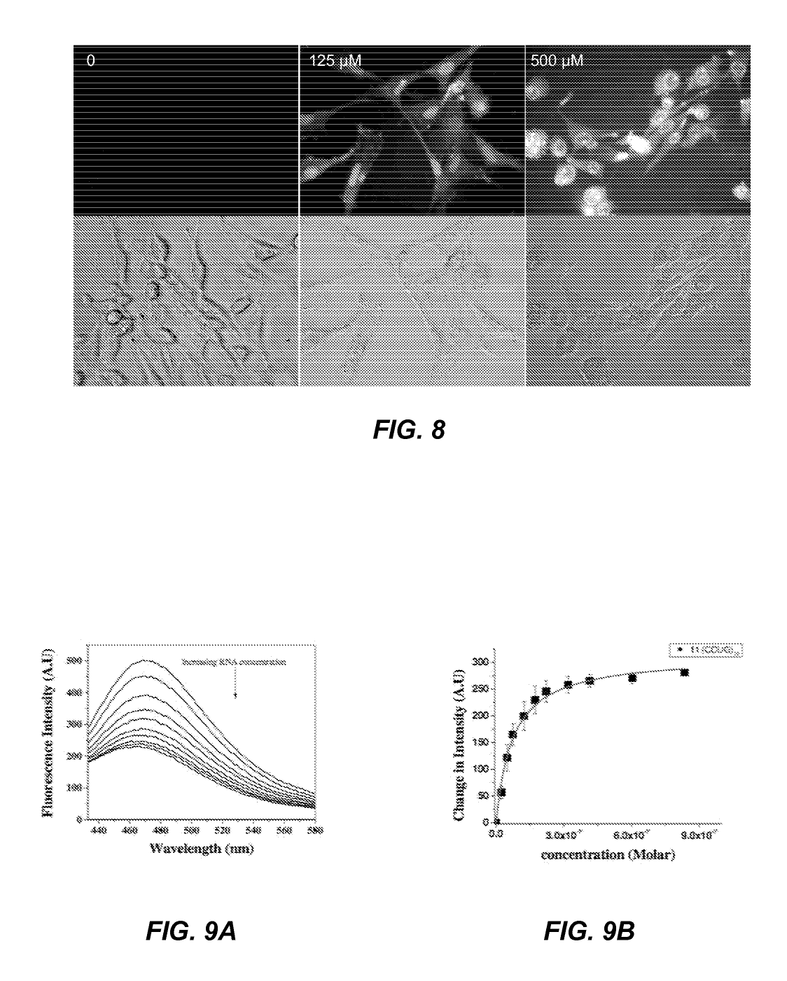

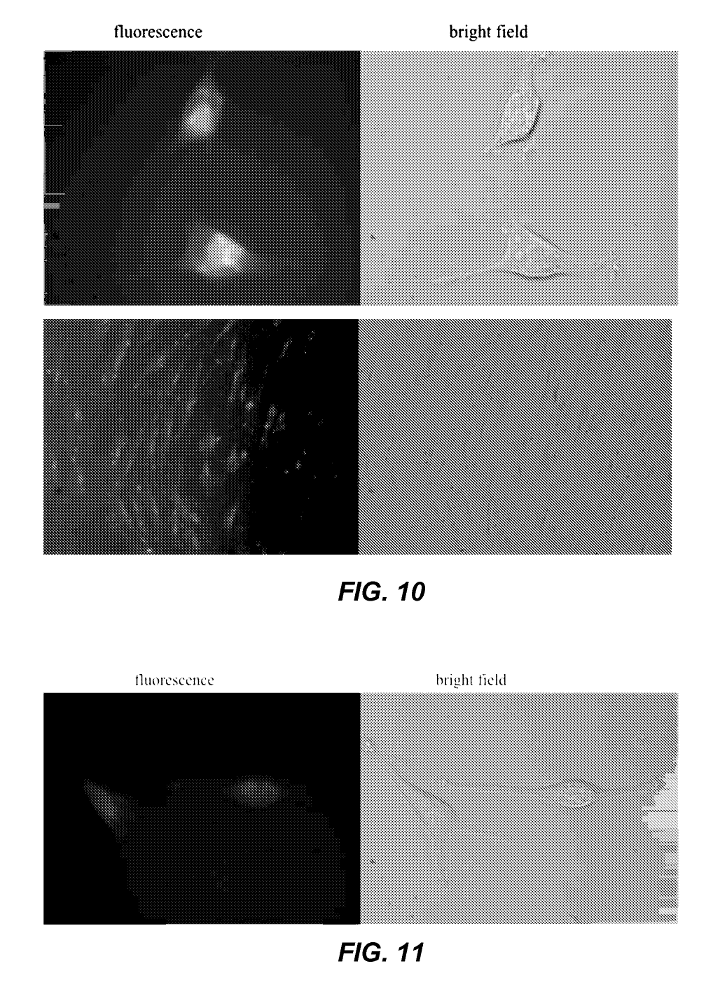

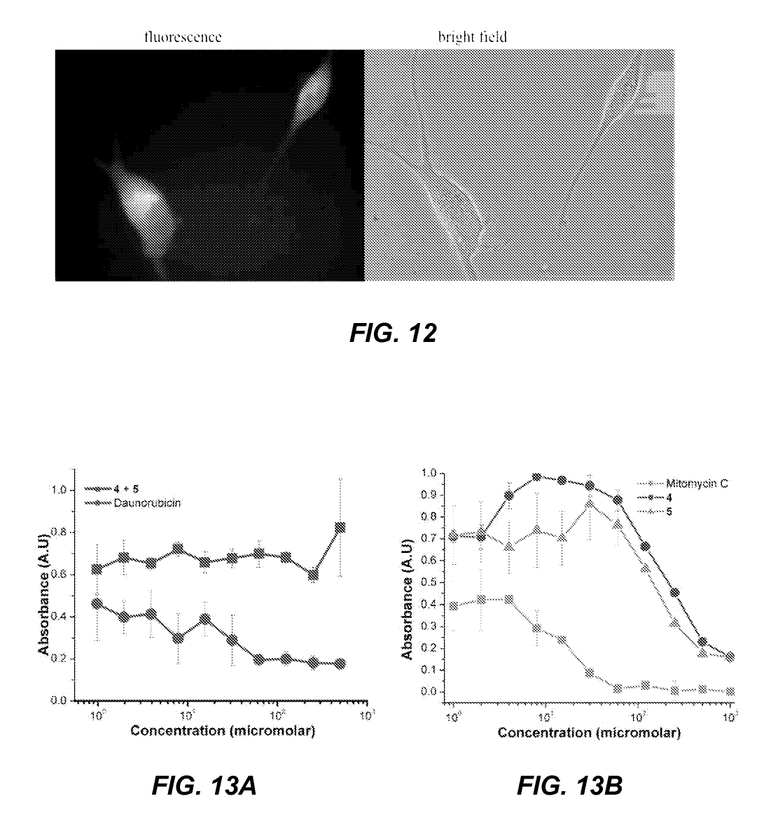

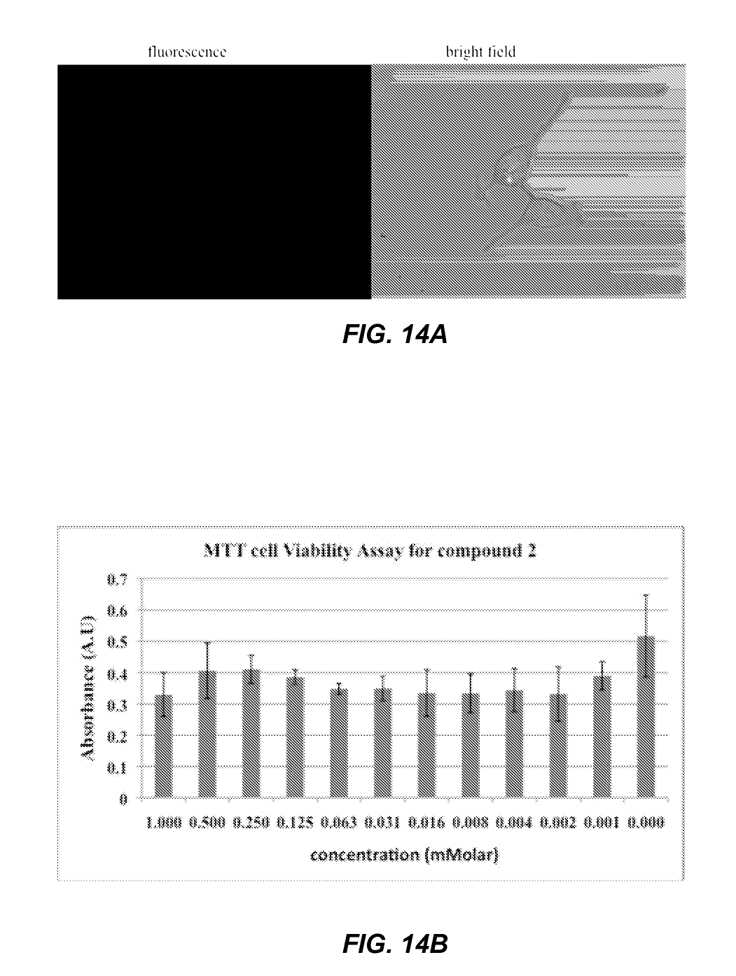

In both cases, penetration of the cell membrane was readily observed because of the fluorescence of the benzo[g]quinoline chromophore; compounds also appear to localize preferentially in the nucleus (FIG. 8). Since the sequestration of MBNL 1 by pathogenic CUG^{exp} RNA occurs entirely in the nucleus, this apparent selective localization even in non CUG^{exp}-containing cell lines is particularly notable. The mechanism of compound entry is not revealed by this analysis. Interestingly, benzo[g]quinoline compound 2 showed only a modest ability to enter cells, and “scrambled” compound 9 was significantly less able to enter cells than compounds 4, 5, or 11 (FIGS. 10-12). Toxicity of compounds 4 and 5 to human fibroblasts (measured as a mixture) and mouse myoblasts was measured by MTT assay (Mosmann, “Rapid Colorimetric Assay for Cellular Growth and Survival: Application to Proliferation and Cytotoxicity Assays,” J. Immunol. Methods 65:55-63 (1983), which is hereby incorporated by reference in its entirety). In fibroblasts, no significant toxicity was observed at concentrations up to 500 μM; increasing toxicity was observed in mouse myoblasts above 100 μM. In contrast, mitomycin C and daunorubicin, commonly employed DNA-targeted cancer chemotherapeutic agents, showed significant toxicity at all concentrations tested (FIGS. 13A-B). Benzo[g]quinoline compound 2 was also tested, and found to have no toxicity in fibroblasts at concentrations up to 500 μM (FIGS. 14A-B), an expected result given its limited ability to cross the cell membrane.

The ability of selected compounds to inhibit the formation of CUG^{exp} RNA-MBNL1 complexes in mouse myoblasts was then evaluated. For this study, C2C12 mouse myoblasts engineered to stably express a firefly luciferase transcript with or without ˜800 uninterrupted CUG repeats in an hDMPK 3′UTR (clones C5-14 and C1-S, respectively) were used. Cells were grown in 100 μl growth media in 96-well plates to ˜10% confluency. Compound was added at various concentrations in triplicate to both C1-S and C₅₋₁₄ cultures, and incubated for 3 days. Culture media was replaced with fresh media containing 1% WST-1 reagent (Roche). After ˜15-20 minutes of incubation, the WST-1 containing media was transferred to a clear 96-well plate, and the absorbances at 450 nm and 690 nm measured with a PerkinElmer EnVision Plate Reader. Cells were gently rinsed (1×PBS) before incubation at −20° C. in 100 μl 1X Passive Lysis Buffer (Promega) for 10 minutes. Immediately before luminescence detection, 20 μl of each lysate was mixed with 50 μl of Luciferase Assay Reagent (Promega) in a fresh opaque-white 96-well plate. Luminescence values were normalized for well-to-well variations in viable cell numbers by dividing by the corresponding (A450 nm-A690 nm) values of the WST-1 containing media.

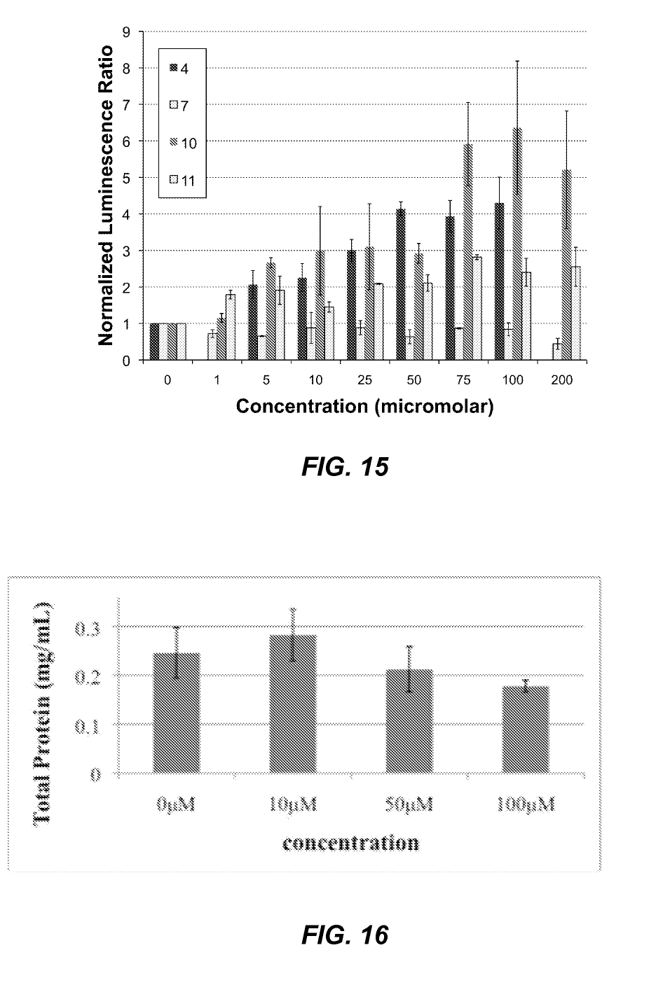

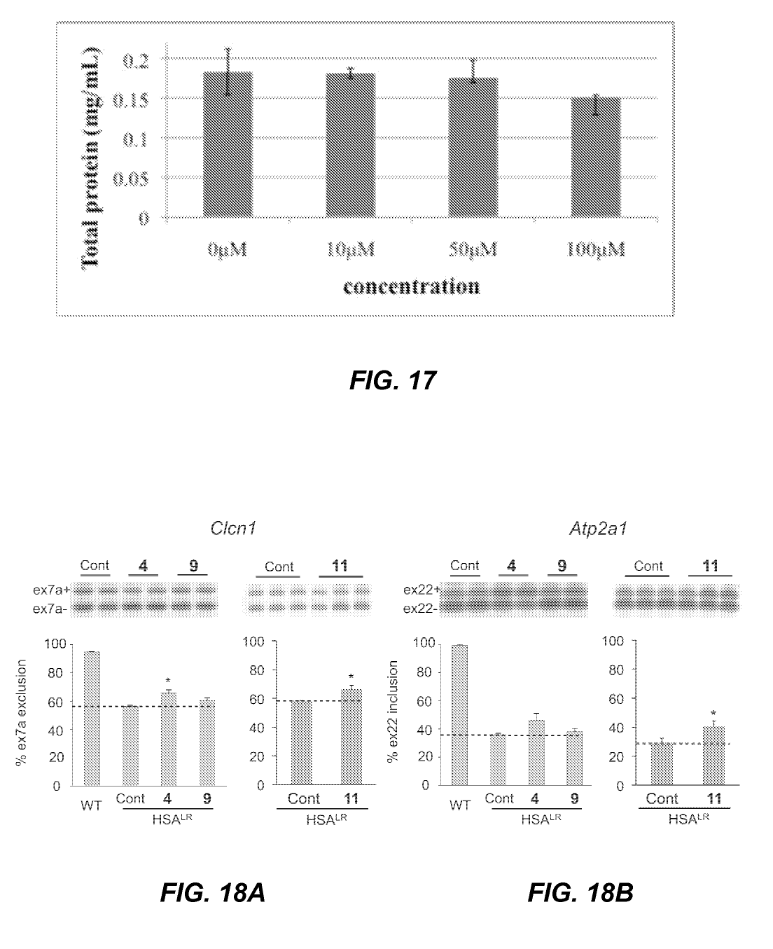

Formation of (CUG)₈₀₀ RNA-MBNL1 complexes in the C₅₋₁₄ cell line suppresses translation of this mRNA and, therefore, suppresses the cellular level of luciferase Inhibition of protein complexation to the (CUG)₈₀₀ RNA allows translation to occur, restoring luciferase expression. A second cell line (C1-S) carrying an analogous luciferase construct lacking the CUG repeats in the 3′-UTR was used as a positive control for luciferase expression. After treatment with a morpholino antisense oligonucleotide (CAG-25) complementary to the CUG repeat RNA, C₅₋₁₄ cells showed an increase in luciferase expression consistent with direct interaction with the (CUG)₈₀₀ mRNA in the nucleus. The increase in luciferase activity is accompanied by a disruption of CUG foci, as seen by fluorescence in-situ hybridization (FISH). It was hypothesized that binding of the compounds of the present invention to the (CUG)₈₀₀ repeat in C₅₋₁₄ cells would likewise promote release of the luciferase transcripts from the nucleus, resulting in increased luciferase expression and activity. In contrast, luciferase activity in positive control (C1-S) cells should not be affected by the presence of CUG RNA binding compounds. Indeed, incubation of various concentrations of compound 4 (0 μM-100 μM) or compound 11 (0 μM-200 μM) with C₅₋₁₄ myoblasts resulted in concentration-dependent increases in luciferase activity, consistent with our hypothesis (FIG. 15). Compound 7 (lacking selectivity to CUG^{exp} RNA) had no effect on luciferase activity when incubated with C₅₋₁₄ cells under the same conditions. No statistically significant change in luciferase signal was observed for C1-S cells (which lack CUG repeats) treated with compounds 10 or 11. However, a concentration-dependent decrease in luciferase activity was observed when these cells were incubated with compound 4. Since the WST-1 viability results showed no observable toxicity of compound 4 to the C1-S cells at the assay concentrations, it was speculated that this effect could result from an off-target effect of compound 4 causing a global decrease in protein synthesis. The concentration of total protein in cell lysates of both C₅₋₁₄ and C1-S cells, as measured by Bradford assay (FIGS. 16-17) indeed showed a decreasing level of protein expression with increasing concentration of compound 4. The fact that this effect is not observed for compounds 10 and 11 likely results from their greater selectivity for CUG repeats. Thus, CUG^{exp} binding compounds are capable of releasing the sequestered mRNA transcripts into the cytoplasm for translation.

### Example 6

### In vivo Efficacy of Dimers in Mouse Myotonic Dystrophy Model

Mouse handling and experimental procedures were conducted in accordance with the Association for Assessment and Accreditation of Laboratory Animal Care. HSA^{LR} transgenic mice in line 20b expressing human skeletal actin RNA with 250 CUG repeats in the 3′ UTR were previously described (Mankodi, “Myotonic Dystrophy in Transgenic Mice Expressing an Expanded CUG Repeat,” Science 289:1769-1772 (2000), which is hereby incorporated by reference in its entirety). Age- and gender-matched HSA^{LR} mice (12-16 weeks old) were injected intraperitoneally with 40 mg/kg of compounds or saline alone once per day for 5 days. Mice were sacrificed one day after the last injection, and vastus muscle was obtained for splicing analysis. RNA extraction, cDNA preparation, and RT-PCR were performed as described previously. The PCR products were electrophoresed on agarose gels for separation, and scanned with a laser fluorimager (Typhoon, GE Healthcare). Quantitative analysis of amplified products was performed by ImageQuant software (Molecular Dynamics). Differences between two groups were evaluated by unpaired Student's t-test.

A critical test of a compound's performance is its ability to function in vivo. DM1 has several well-studied mouse models (Gomes-Pereira et al., “Myotonic Dystrophy Mouse Models: Towards Rational Therapy Development,” Trends Mol. Med. 17:506-517 (2011), which is hereby incorporated by reference in its entirety). Compound activity in the HSA^{LR} mouse model, in which transgenic mice carry a long CTG repeat inserted into the human skeletal actin (HSA) gene in skeletal muscle (Mankodi, “Myotonic Dystrophy in Transgenic Mice Expressing an Expanded CUG Repeat,” Science 289:1769-1772 (2000), which is hereby incorporated by reference in its entirety) was examined. These mice exhibit several DM1-like phenotypic characteristics. They also display aberrant splicing of genes, including Clcn1 and Atp2a1, which are hallmarks of nuclear transcript sequestration.

A modest but statistically significant (P=0.0115) improvement of Clcn1 splicing following treatment with compound 4, but not compound 9 was observed (FIG. 18). Compound 4 also qualitatively appeared to restore splicing in the Atp2a1 gene, but the presence of one nonresponder prevented this from being statistically significant. Statistically significant improvements in both Clcn1 and Atp2a1 (P=0.0113 and P=0.0230, respectively) were observed following treatment with compound 11 (as experiments were done separately, separate control mice were required). The observation that activity of compounds in vivo correlated with their ability to bind (CUG^{exp})RNA is not only gratifying, but it also confirms the predictive value of in vitro (CUG^{exp}) binding models for predicting in vivo activity.

It was also observed that mice treated with compound 4 exhibited significant acute toxicity effects while those administered with compound 11 did not. It is tempting to relate these differences in toxicity to the higher (CUG^{exp}) selectivity of compound 11; however, the complexity of factors contributing to selectivity is sufficiently high so as to make this an unwise extrapolation. The amount of splicing activity restored is similar to that produced by pentamidine (Warf et al., “Pentamidine Reverses the Splicing Defects Associated with Myotonic Dystrophy,” Proc. Natl. Acad. Sci. U.S.A. 10618551-18556 (2009), which is hereby incorporated by reference in its entirety) or intramuscular injection of CUG-disrupting peptides (Garcia-Lopez et al., “In Vivo Discovery of a Peptide that Prevents CUG-RNA Hairpin Formation and Reverses RNA Toxicity in Myotonic Dystrophy Models,” Proc. Natl. Acad. Sci. U.S.A. 108:11866-11871 (2011), which is hereby incorporated by reference in its entirety), but it is anticipated the greater selectivity of the compounds described (particularly compound 11) may provide a more favorable pathway for future development.

### Discussion of Examples 1-6

Using a moderate-affinity ligand identified from a resin-bound dynamic combinatorial library as a starting point, next-generation lead compounds able to selectively bind DM1 and DM2 RNA with high affinity have been developed. These compounds represent the first use of the benzo[g]quinoline moiety in an RNA-binding context, an important advance in that this substructure allows for direct visualization of compounds in cells via fluorescence. The selectivity of compound 11 for CUG repeats over CCUG repeats is particularly notable, as is its enhanced affinity for longer CUG repeat sequences. Selectivity for longer repeats is potentially highly advantageous. Since isolated CUG trinucleotides and short repeat sequences are found throughout the transcriptome, binders must differentiate between these and longer repeats. Several of the compounds synthesized show very low toxicity in human fibroblast and mouse myoblast cell lines, suggesting the high sequence selectivity displayed by these structures is transferable to a biological context. Compounds examined in this work are able to release the nuclear retention of a CUG repeat-containing transcript in a concentration-dependent manner in mouse myoblasts, and improve splicing in a mouse model of DM1. Thus, Dynamic Combinatorial Chemistry can serve as a starting point from which to develop high-affinity sequence-selective RNA binding compounds with desirable biological activity ex vivo (i.e., in cell culture) and in vivo (in mice).

### Example 7

### Synthesis Compound 17 (Disulfide) and Analog Compounds 18, 19 (Olefin)

Compound 17 (FIG. 20A) was synthesized using the solid phase peptide synthesis procedure described in Example 2 except using Phe, Pro, and Cys(Trt)-OH to form resin-bound monomer. After washing, the resin-bound monomer was cleaved by the addition of 50% TFA/1% TES/DCM for 1 hour, and ether precipitated. The cleaved monomer was dried overnight under vacuum followed by HPLC purification. About 10 mg of the pure monomer was weighed into a glass vial equipped with a magnetic stirrer and 2 ml of doubly distilled H₂O was added to dissolve it. Next, two drops of DMSO was added and the mixture was stirred for 72 hours at room temperature. Disulfide-linked dimer formation was monitored by reverse phase analytical HPLC. The final dimer product was isolate and purified by reversed phase preparative HPLC described supra.

Compounds 18 (E isomer) and 19 (Z isomer) (FIG. 21A) were synthesized using the solid phase peptide synthesis procedure described in Example 2 except using Phe, Pro, and Allylglycine to form resin-bound monomer. Dimerization was achieved using the process illustrated in Scheme 2 (Example 2), and the isomers (E and Z ratio of 3:2 respectively) were separated using preparative reversed-phase-HPLC on a C18 column (Waters, XBridge™ Prep C18 5 μm OBD™, 19×250 mm) using a water-acetonitrile gradient with 0.1% TFA.

### Example 8

### Analysis of Dimer Binding Affinity to HIV-1 FSS and In Vitro Studies

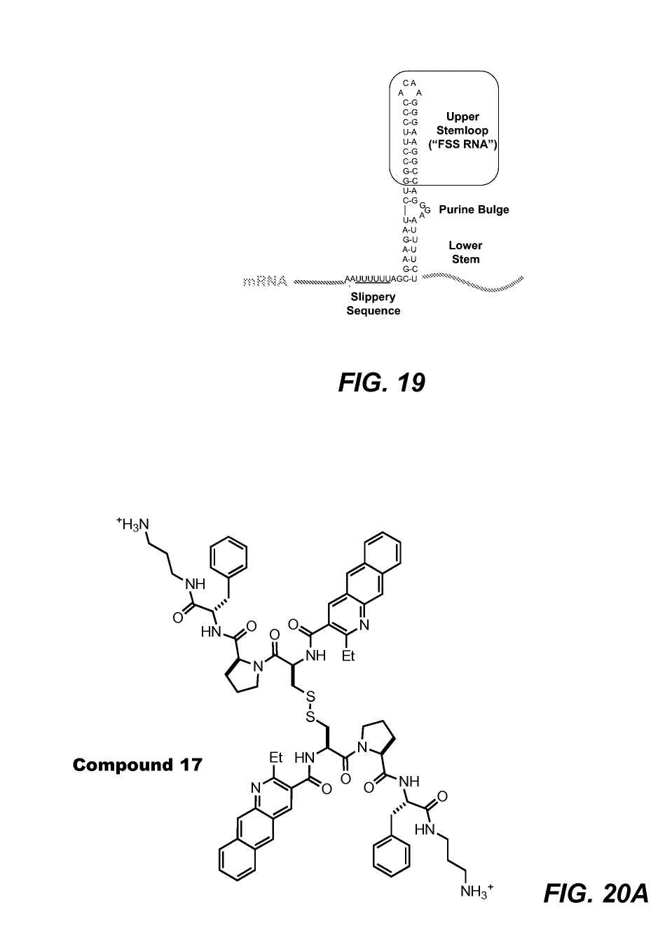

SPR and fluorescence titration experiments, performed using the protocol described in Examples 3 and 4 above, except using as the target a highly conserved RNA sequence responsible for regulating the production of the Gag-Pol polyprotein via a −1 ribosomal frameshift, which occurs along a “slippery sequence” in gag-pol mRNA (SEQ ID NO: 7). This “frameshift stimulatory sequence” (or “HIV-1 FSS”) consists of an upper stemloop and a lower stem, separated by a purine bulge in the most common types of HIV (FIG. 19). For the fluorescent titration experiments, the upper stemloop of SEQ ID NO: 7 was employed. Cell uptake and toxicity studies were carried out using human fibroblasts according to Example 5 above.

Compound 17 molecule does indeed have enhanced affinity for the HIV-1 FSS (K_{D}=741±0.54 nM, as measured by SPR). Moreover, by virtue of the benzo[g]quinoline moiety, the structure has strong visible fluorescence (FIGS. 20B-C). Compound 17 retains selectivity for the HIV-1 FSS, in that it cannot be competed off by excess yeast tRNA or total yeast RNA.

Compound 18 molecule binds the HIV-1 FSS with high affinity (K_{D}=89.0±0.005 nM and 102.0±0.021 nM for E and Z isomers respectively, as measured by SPR). Moreover, compound 18 (E-isomer) readily penetrates cell membranes (FIGS. 21B-C; note that this is direct imaging of the intrinsic fluorescence of compound 18), and as measured by MTT assay (Mosmann, “Rapid Colorimetric Assay for Cellular Growth and Survival: Application to Proliferation and Cytotoxicity Assays,” J. Immunol. Meth. 65:55-63 (1983), which is hereby incorporated by reference in its entirety) is essentially non-toxic to human fibroblasts at concentrations up to 500 micromolar (FIG. 21C).

### Example 9

### Assessment of Compound 18 (E-Isomer) Against Pseudotyped HIV

Initial experiments were conducted to ascertain if compound 14 (designated as compound 5-5 Z in PCT Publ. No. WO 2009/015384 to Miller et al., which is hereby incorporated by reference in its entirety) and compound 18 (Example 7 above) have any effect on the infectivity and replicative ability of pseudotyped HIV. This assay utilizes HEK293T cells co-transfected with VSV-G and viral DNA vector pDHIV3-GFP as the viral producer cell line (Andersen et al., “HIV-1 Vpr-induced Apoptosis is Cell Cycle Dependent and Requires Bax but not ANT,” PLoS Pathogens 2:1106-1118 (2006), which is hereby incorporated by reference in its entirety). This vector is derived from HIV-1_{NL4-3}, lacking only the env and nef genes (gag and pol, and the frameshift required for production of the Gag-Pol polyprotein, are preserved). After transfection, cells were incubated with compound for 20 hours. Viruses produced in untreated and treated cells were recovered from the media and viral titers were assessed using a p24 ELISA assay as described previously by Miller et al., “The Dimerization Domain of HIV-1 Viral Infectivity Factor Vif is Required to Block Virion Incorporation of APOBEC3G,” Retrovirology 4:81 (2007), which is hereby incorporated by reference in its entirety. The same number of virus from control and compound-treated cells were used to infect a HeLa cell line expressing luciferase under the control of an LTR promoter (Platt et al., “Effects of CCRS and CD4 Cell Surface Concentrations on Infections by Macrophagetropic Isolates of Human Immunodeficiency Virus Type 1,” J. Virol. 72:2855-2864 (1998), which is hereby incorporated by reference in its entirety). Viral infectivity was quantified in microtiter well format based on the luciferase signals. As shown in FIG. 22, both compound 14 and compound 18 produce a significant (24%-29%) and reproducible decrease in viral titer vs. control at a concentration of 500 nM. In contrast, 2-ethyl benzo[g]quinoline carboxylic acid (compound 2) has no effect at 500 nM.

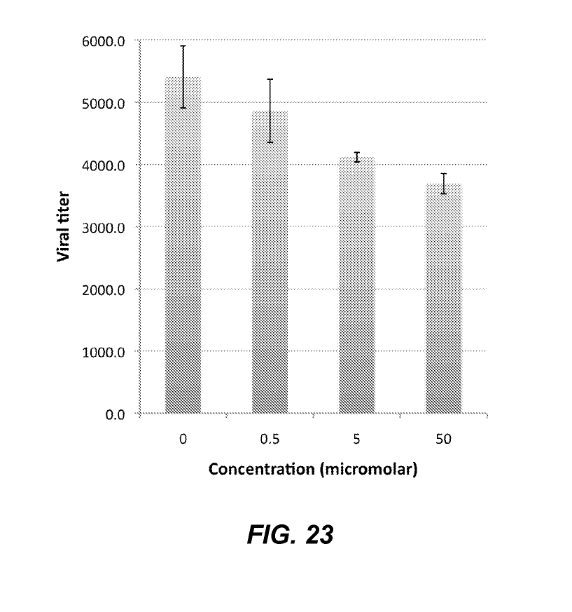

This experiment was repeated using a dose-response study to assess the efficacy of compound 18. Viral counts used for infection were normalized to p24 by ELISA (confirmed by Western blots of virus spun through a sucrose column). Cells were plated at 10,000 cells per well, and dosed with compound 18 at 4 hours and 20 hours post-transfection. Virus was harvested 25 hours post-transfection. Actin concentration (as measured by Western blot) was used to correct viral titers for any subtle variation in cell number, and to confirm that the observed effect is specific to virus production and not to off-target effects. Small differences in absolute viral titer for the control (no compound) relative to previously described data are the result of a slightly different media volume used in this assay. Importantly, the clear dose-response relationship observed (FIG. 23) provides additional strong support for the further development of compound 18 and the next-generation analogs. It is particularly noted that since the pseudotyped HIV assay only allows for one round of replication, it is likely that compound 18 will show even stronger activity in viral spreading assays with “native” HIV.

### Example 10

### Synthesis and In Vitro Testing N-Methyl Analogs of Compounds 4, 5

Peptide N-methylation is a strategy that has been successfully employed for some time as a means to enhance peptide-receptor affinity via conformational restriction (Holladay et al., “Tetrapeptide CCK-a Agonists: Effect of Backbone N-Methylations on in-vitro and in-vivo CCK Activity,” J. Med. Chem. 37:630-635 (1994), which is hereby incorporated by reference in its entirety), and more recently has found favor as a method for enhancing the bioavailability and biostability of peptides. The latter has been driven particularly by the Kessler group, who observed that some naturally occurring N-methylated peptide drugs such as cyclosporine are orally bioavailable, despite violating the entire set of Lipinski guidelines for “drug like” compounds (Chatterjee et al., “N-methylation of Peptides: A New Perspective in Medicinal Chemistry,” Acc. Chem. Res. 41:113-1342 (2008), which is hereby incorporated by reference in its entirety).

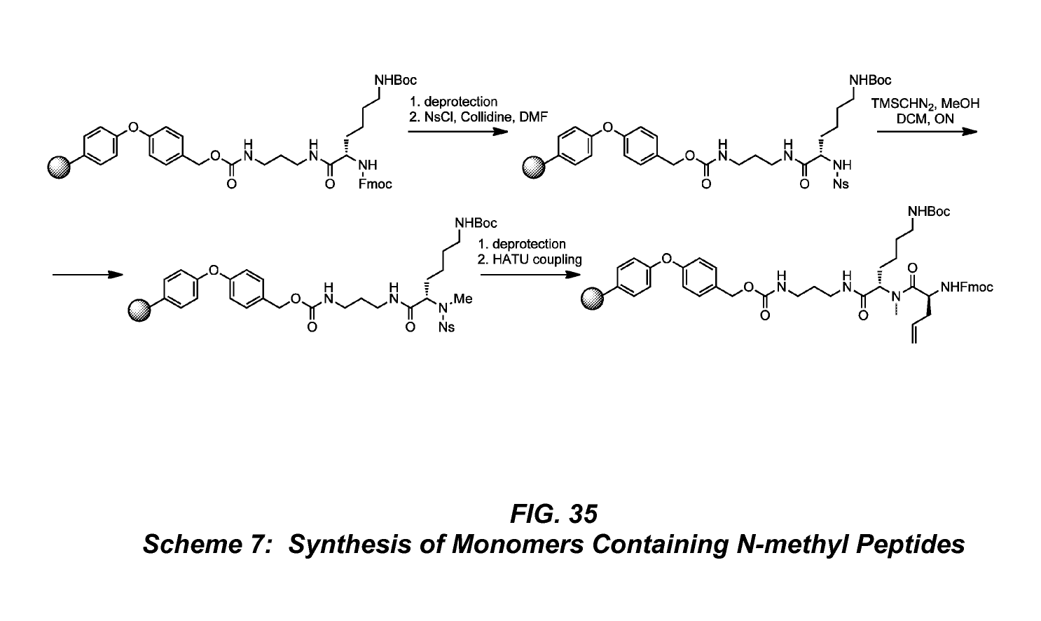

Compounds 20-22, containing one or more N-methyl amino acid residues, were synthesized using a protocol similar to that shown in Scheme 7 (FIG. 35).

Compound 20: Wang resin (1.0 g, 100-200μ mesh) was activated with 1,1′-carbonyldiimidazole (DIC, 1.62 g, 10 mmol) in 15 mL of DMF for 12 h on a LabQuake rotator. The resin was then washed three times each with DMF, CH₂Cl₂ and again with DMF, followed by reaction with 1,3-diaminopropane (832 μL, 10 mmol) in DMF for another 12 h. After repeating the wash cycle, the first amino acid (Fmoc-Lys(Boc)-OH, 3 mmol) was coupled to the resin using HBTU (1.14 g, 3 mmol) and DIPEA (0.85 mL, 5 mmol) in 15 mL DMF and rotating the mixture for 1 h. Following the wash cycle, Fmoc deprotection was accomplished using 15 mL of 20% piperidine in DMF for 1 hour, followed again by the wash cycle. Nosyl chloride (0.7 g, 3.0 mmol) was then added, followed by the addition of collidine (666 μL, 5.0 mmol). Reaction was allowed to proceed for 2 h. The resin was then washed three times each with DCM, DMF, dry DCM and then transferred into 50 mL round bottom flask, fitted with rubber septa and a venting needle. Resin was resuspended in dry DCM. (Trimethylsilyl)diazomethane (2.0 M in ether, 4.6 mL, 9.0 mmol) was added followed by MeOH (0.4 mL, 13.0 mmol). Reaction was allowed to proceed overnight (flask was agitated on an orbital shaker during the reaction). Resin was transferred into a standard solid-phase reaction vessel with filtering and washed with DCM, MeOH, DCM, DMF and resuspended in DMF. (Small portion of the peptide was cleaved from the resin to monitor the progress of the reaction. If reaction was not complete, reaction with (trimethylsilyl)diazomethane was repeated). DBU (747 μL, 5.0 mmol) was then added, followed by the addition of β-mercaptoethanol (708 μL, 10.0 mmol). The deprotection reaction was allowed to proceed on the LabQuake rotator for 1 h, washed with DMF (×6) and then resuspended in DMF. Fmoc-L-allylglycine (0.67 g, 2.0 mmol) was then added, followed by the addition of HATU (0.78 g, 2.0 mmol), DIPEA (697 μL, 4.0 mmol). The coupling reaction was allowed to proceed on the LabQuake rotator for 2 h, washed with DCM (×3), DMF (×3), and then deprotected for 1 h using 20% piperidine/DMF solution (15 mL). After repeating the wash cycle, Fmoc-L-proline (3 mmol) was coupled to the resin using HBTU (1.14 g, 3 mmol) and DIPEA (0.87 mL, 5 mmol) in 15 mL DMF and rotating the mixture for 1 h. Following the wash cycle, Fmoc deprotection was accomplished using 15 mL of 20% piperidine in DMF for 1 hour, followed again by the wash cycle. Lastly, 2-ethyl benzo[g]quinoline carboxylic acid hydrochloride (1.5 mmol) was coupled to the resin using HBTU (0.57 g, 1.5 mmol) and DIPEA (0.7 mL, 4 mmol) in 15 mL DMF and rotating the mixture for 12 h, then washed with DMF (×3) and DCM (×3). Next, the resin was split into two equal parts of 0.50 g. One part was cleaved with 30% TFA/1% TES in 10 mL of CH₂Cl₂ for 1 h. Compounds 20E and 20Z (3:1 isomer ratio) were prepared using metathesis reaction and separated using preparative reversed-phase-HPLC, using the same protocol as for the synthesis of compounds 4 and 5.

Compound 21: Wang resin (1.0 g, 100-200μ mesh) was activated with 1,1′-carbonyldiimidazole (DIC, 1.62 g, 10 mmol) in 15 mL of DMF for 12 h on a LabQuake rotator. The resin was then washed three times each with DMF, CH₂Cl₂ and again with DMF, followed by reaction with 1,3-diaminopropane (832 μL, 10 mmol) in DMF for another 12 h. After repeating the wash cycle, the first amino acid (Fmoc-Lys(Boc)-OH, 3 mmol) was coupled to the resin using HBTU (0.76 g, 2 mmol) and DIPEA (0.7 mL, 4 mmol) in 15 mL DMF and rotating the mixture for 1 h. Following the wash cycle, Fmoc deprotection was accomplished using 15 mL of 20% piperidine in DMF for 1 hour, followed again by the wash cycle. Fmoc-L-allylglycine (0.67 g, 2.0 mmol) was coupled to the resin using HBTU (1.14 g, 3 mmol) and DIPEA (0.85 mL, 5 mmol) in 15 mL DMF and rotating the mixture for 1 h. Following the wash cycle, Fmoc deprotection was accomplished using 15 mL of 20% piperidine in DMF for 1 hour, followed again by the wash cycle. Nosyl chloride (0.7 g, 3.0 mmol) was then added, followed by the addition of collidine (666 μL, 5.0 mmol). Reaction was allowed to proceed for 2 h. The resin was then washed three times each with DCM, DMF, dry DCM and then transferred into 50 mL round bottom flask, fitted with rubber septa and a venting needle. Resin was resuspended in dry DCM. (Trimethylsilyl)diazomethane (2.0 M in ether, 4.6 mL, 9.0 mmol) was added followed by MeOH (0.4 mL, 13.0 mmol). Reaction was allowed to proceed overnight (flask was agitated on an orbital shaker during the reaction). Resin was transferred into a standard solid-phase reaction vessel with filtering and washed with DCM, MeOH, DCM, and DMF and resuspended in DMF. (Small portion of the peptide was cleaved from the resin to monitor the progress of the reaction. If reaction was not complete, reaction with (trimethylsilyl)diazomethane was repeated). DBU (747 μL, 5.0 mmol) was then added, followed by the addition of β-mercaptoethanol (708 μL, 10.0 mmol). The deprotection reaction was allowed to proceed on the LabQuake rotator for 1 h, washed with DMF (×6) and then resuspended in DMF. Fmoc-L-proline (3 mmol) was coupled to the resin using HATU (1.16 g, 3 mmol) and DIPEA (0.87 mL, 5 mmol) in 15 mL DMF and rotating the mixture for 2 h. Following the wash cycle, Fmoc deprotection was accomplished using 15 mL of 20% piperidine in DMF for 1 hour, followed again by the wash cycle. Lastly, 2-ethyl benzo[g]quinoline carboxylic acid hydrochloride (1.5 mmol) was coupled to the resin using HBTU (0.57 g, 1.5 mmol) and DIPEA (0.7 mL, 4 mmol) in 15 mL DMF and rotating the mixture for 12 h, then washed with DMF (×3) and DCM (×3). Next, the resin was split into two equal parts of 0.50 g. One part was cleaved with 30% TFA/1% TES in 10 mL of CH₂Cl₂ for 1 h. Compounds 21E and 21Z (2:1 isomer ratio) were prepared using metathesis reaction and separated using preparative reversed-phase-HPLC, using the same protocol as for the synthesis of compounds 4 and 5.

Compound 22: Wang resin (1.0 g, 100-200μ mesh) was activated with 1,1′-carbonyldiimidazole (DIC, 1.62 g, 10 mmol) in 15 mL of DMF for 12 h on a LabQuake rotator. The resin was then washed three times each with DMF, CH₂Cl₂ and again with DMF, followed by reaction with 1,3-diaminopropane (832 μL, 10 mmol) in DMF for another 12 h. After repeating the wash cycle, the first amino acid (Fmoc-Lys(Boc)-OH, 3 mmol) was coupled to the resin using HBTU (1.14 g, 3 mmol) and DIPEA (0.85 mL, 5 mmol) in 15 mL DMF and rotating the mixture for 1 h. Following the wash cycle, Fmoc deprotection was accomplished using 15 mL of 20% piperidine in DMF for 1 hour, followed again by the wash cycle. Nosyl chloride (0.7 g, 3.0 mmol) was then added, followed by the addition of collidine (666 μL, 5.0 mmol). Reaction was allowed to proceed for 2 h. The resin was then washed three times each with DCM, DMF, and dry DCM and then transferred into 50 mL round bottom flask, fitted with rubber septa and a venting needle. Resin was resuspended in dry DCM. (Trimethylsilyl)diazomethane (2.0 M in ether, 4.6 mL, 9.0 mmol) was added followed by MeOH (0.4 mL, 13.0 mmol). Reaction was allowed to proceed overnight (flask was agitated on an orbital shaker during the reaction). Resin was transferred into a standard solid-phase reaction vessel with filtering and washed with DCM, MeOH, DCM, and DMF and resuspended in DMF. (Small portion of the peptide was cleaved from the resin to monitor the progress of the reaction. If reaction was not complete, reaction with (trimethylsilyl)diazomethane was repeated). DBU (747 μL, 5.0 mmol) was then added, followed by the addition of β-mercaptoethanol (708 μL, 10.0 mmol). The deprotection reaction was allowed to proceed on the LabQuake rotator for 1 h, washed with DMF (×6), and then resuspended in DMF. Fmoc-L-allylglycine (0.67 g, 2.0 mmol) was then added, followed by the addition of HATU (0.78 g, 2.0 mmol, 2 eq), DIPEA (697 μL, 4.0 mmol, 4 eq). The coupling reaction was allowed to proceed on the LabQuake rotator for 2 h, washed with DCM (×3), DMF (×3), and resuspended in DMF. Nosyl chloride (0.7 g, 3.0 mmol) was then added, followed by the addition of collidine (666 μL, 5.0 mmol). Reaction was allowed to proceed for 2 h. The resin was then washed three times each with DCM, DMF, and dry DCM and then transferred into 50 mL round bottom flask, fitted with rubber septa and a venting needle. Resin was resuspended in dry DCM. (Trimethylsilyl)diazomethane (2.0 M in ether, 4.6 mL, 9.0 mmol) was added followed by MeOH (0.4 mL, 13.0 mmol). Reaction was allowed to proceed overnight (flask was agitated on an orbital shaker during the reaction). Resin was transferred into a standard solid-phase reaction vessel with filtering and washed with DCM, MeOH, DCM, and DMF and resuspended in DMF. (Small portion of the peptide was cleaved from the resin to monitor the progress of the reaction. If reaction was not complete, reaction with (trimethylsilyl)diazomethane was repeated). DBU (747 mL, 5.0 mmol) was then added, followed by the addition of β-mercaptoethanol (708 mL, 10.0 mmol). The deprotection reaction was allowed to proceed on the LabQuake rotator for 1 h, washed with DMF (×6) and then resuspended in DMF. Fmoc-L-proline (3 mmol) was coupled to the resin using HATU (1.16 g, 3 mmol) and DIPEA (0.87 mL, 5 mmol) in 15 mL DMF and rotating the mixture for 2 h. Following the wash cycle, Fmoc deprotection was accomplished using 15 mL of 20% piperidine in DMF for 1 hour, followed again by the wash cycle. Lastly, 2-ethyl benzo[g]quinoline carboxylic acid hydrochloride (1.5 mmol) was coupled to the resin using HBTU (0.57 g, 1.5 mmol) and DIPEA (0.7 mL, 4 mmol) in 15 mL DMF and rotating the mixture for 12 h, then washed with DMF (×3) and DCM (×3). Next, the resin was split into two equal parts of 0.50 g. One part was cleaved with 30% TFA/1% TES in 10 mL of CH₂Cl₂ for 1 h. Compounds 22E and 22Z (3:2 isomer ratio) were prepared using metathesis reaction and separated using preparative reversed-phase-HPLC, using the same protocol as for the synthesis of compounds 4 and 5.

Binding affinities of N-methyl compounds 20 (E), 21(Z), 22(E), and 22(Z) were performed as described in Example 3 except that only (CUG)₂ and (CUG)₁₀ were used as targets. The binding affinities are shown in Table 7 below. Binding affinities of compounds 4 and 5 are reproduced for comparison.

TABLE 7
Binding Constant Data for N-methyl Compounds

| RNA | Compound | | | |
|---|---|---|---|---|
| Target | 4 (Z) | 5 (E) | 20 (E) | 20 (Z) |
| (CUG)₁₀ | K_{D} = 45 ± 3 nM | K_{D} = 88 ± 1 nM | K_{D} = 83 ± 9 nM | ND |
| | kₐ = 2.78E+4/M * s | kₐ = 2.84E+4/M * s | kₐ = 1.8 ± 0.9E+4/M * s | |
| | k_{d} = 1.18E−3/s | k_{d} = 2.47E−3/s | k_{d} = 1.4 ± 0.6E−3/s | |
| (CUG)₂ | k_{d} = 49 ± 2 nM | K_{d} = 50 ± 2 nM | ND | ND |
| | kₐ = 2.45E+4/M * s | kₐ = 2.01E+4/M * s | | |
| | k_{d} = 8.83E−4/s | k_{d} = 7.69E−4/s | | |

| RNA | Compound | | | |
|---|---|---|---|---|
| Target | 21 (E) | 21 (Z) | 22 (E) | 22 (Z) |
| (CUG)₁₀ | ND | K_{D} = 102 ± 10 nM | K_{D} = 72 ± 13 nM | K_{D} = 378 ± 180 nM |
| | | kₐ = 1.6 ± 0.3E+4/M * s | bkₐ = 1.2 ± 0.3E+4/M * s | kₐ = 9.2 ± 0.4E+3/M * s |
| | | k_{d} = 1.6 ± 0.1E−3/s | k_{d} = 8.7 ± 0.3E−4/s | k_{d} = 3 ± 2E−3/s |
| (CUG)₂ | ND | ND | k_{d} = 42 ± 8 nM | ND |
| | | | kₐ = 1.9 ± 0.4E+4/M * s | |
| | | | k_{d} = 8 ± 4E−4/s | |

ND = Not Determined

All tested N-methylated analogs showed similar affinity for (CUG)₁₀ and (CUG)₂ RNA as their parental compounds 4 and 5, proving that introduction of N-methylated amino acids into the peptide structure do not significantly alter the binding constants. Affinity of the compound 22E for (CUG)₂ RNA appeared to strengthen slightly, showing the same trend as was observed for compound 5E.

Other combinations of the singly and doubly N-methylated monomers will be preapared, purified, and screened for their binding affinities for HIV-1 FSS. Dimers with high affinity will be tested for their efficacy inhibiting the infectivity and replicative ability of pseudotyped HIV as described in Example 9 above.

### Example 11

### Synthesis of Hetero-Dimers Containing Single Monomer from Compound 18

Experiments thus far demonstrate that affinity for the HIV-1 FSS largely arises from the planar heterocyclic (quinoline or benzo[g]quinoline) moiety, while specificity derives primarily from the peptide. To determine whether removal of one of the putative intercalators will yield a compound with similar affinity and selectivity, or potentially better, i.e., if binding by the two is anticooperative, two pairs of isomers will be synthesized by replacing one benzo[g]quinoline with either a pyridine (Compounds 30 (E) and 31 (Z), incapable of intercalating but still potentially able to engage in other binding interactions) and Compounds 32 (E) and 33 (Z), which delete the heterocycle entirely. In addition to providing information about the requirements for binding, these compounds would also have the advantage of a modest reduction in molecular weight.

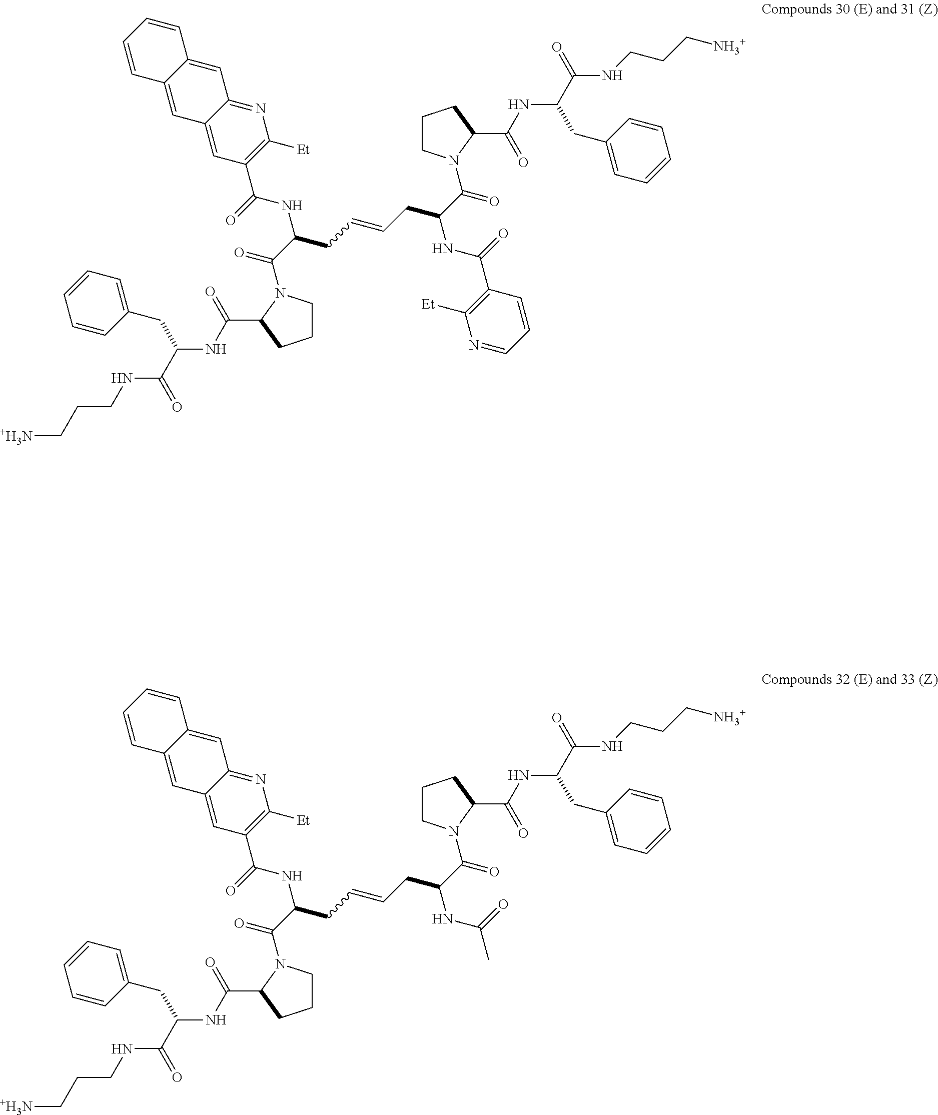

These four new hetero-dimers will be prepared, purified, and screened for their binding affinities for HIV-1 FSS. Dimers with high affinity will be tested for their efficacy inhibiting the infectivity and replicative ability of pseudotyped HIV as described in Example 9 above.

### Example 12

### Synthesis of Reduced Amide Analogs of Compound 18

Another approach for increasing the biostability of lead compounds is the conversion of one or more peptide bonds to a reduced analog, termed a “pseudopeptide” (Zivec et al., “Recent Advances in the Synthesis and Applications of Reduced Amide Pseudopeptides,” Curr. Med. Chem. 16:2289-2304 (2009), which is hereby incorporated by reference in its entirety). A small subset of peptides to which this method has been successfully applied includes ligands for MHC-II (Cotton et al., “Pseudopeptide Ligands for MHC II-restricted T Cells,” Int. Immunol. 10:159-166 (1998), which is hereby incorporated by reference in its entirety), bactericidal peptides (Oh and Lee, “Characterization of the Unique Function of a Reduced Amide Bond in a Cytolytic Peptide that Acts on Phospholipid Membranes,” Biochem. J. 352:659-666 (2000), which is hereby incorporated by reference in its entirety), and neurotensin 8-13 mimetics (Wustrow et al., “Reduced Amide Bond Neurotensin 8-13 Mimetics with Potent in vivo Activity,” Bioorg. Med. Chem. Lett. 5:997-1002 (1995), which is hereby incorporated by reference in its entirety.). Like the peptide N-methylation strategy, conversion of peptide bonds to pseudopeptides changes both conformational dynamics (loss of peptide bond conformational restriction) and charge (conversion of neutral amide NH to protonated amine) To test this strategy in the context of our HIV-1 FSS binding compounds, we will synthesize 34 and 35 (symmetrical incorporation of pseudopeptide phenylalanine analogs), and 36 and 37 (symmetrical incorporation of Pro-Phe pseudopeptides). In all four cases, the diaminopropane moiety will be deleted, as it is believed this moiety provides only a charge-based contribution to binding rather than specific interactions.

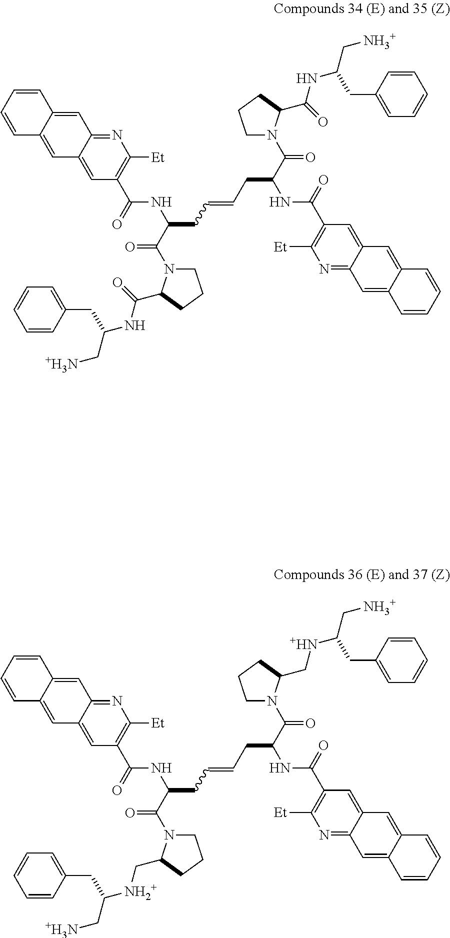

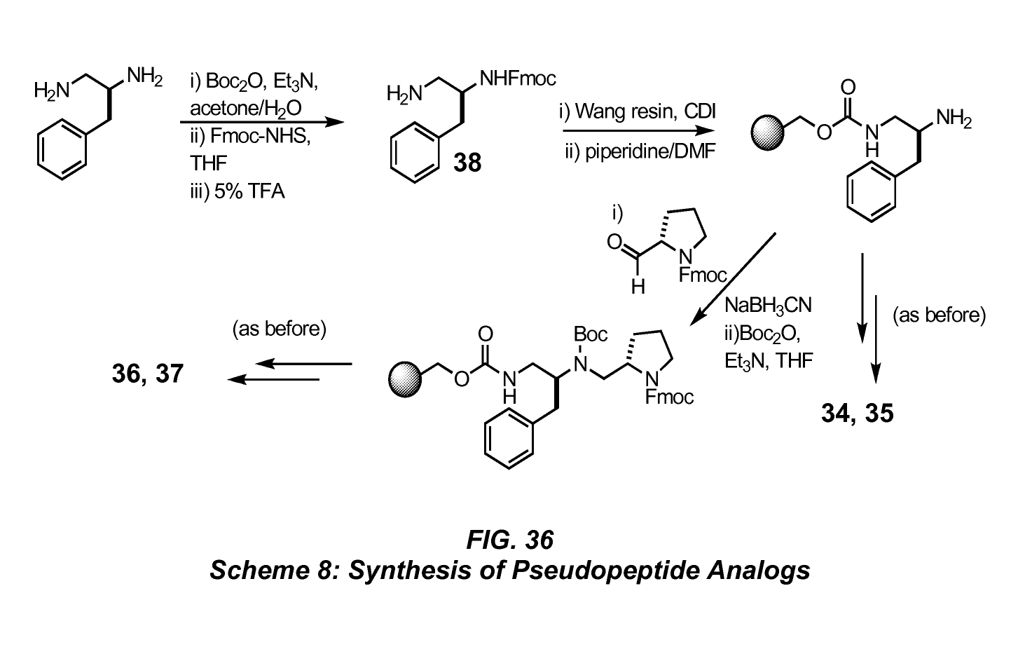

Synthesis of 34-36 will begin with commercially available S-3-phenyl 1,2,-propane diamine; selective Boc protection of the primary amine followed by Fmoc protection of the secondary amine and removal of Boc will provide 28, suitable for CDI-mediated coupling to Wang resin as shown in Scheme 8 (FIG. 36). Alternatively, if the selective protection/deprotection scheme to produce 38 proves problematic, it may also be prepared via a 5-step literature procedure from Fmoc-Phenylalanine (Sureshbabu et al., “N-Urethane-protected Amino Alkyl Isothiocyanates: Synthesis, Isolation, Characterization, and Application to the Synthesis of Thioureidopeptides,” J. Org. Chem. 74:5260-5266 (2009), which is hereby incorporated by reference in its entirety). The remainder of the synthesis of 34 and 35 will follow our previously developed procedures. For the synthesis of 36 and 37, Fmoc-deprotection of resin-immobilized S-3-phenyl 1,2-propane diamine will be followed by reductive amination with Fmoc-(S)-pyrrolidine-2-carbaldehyde following established procedures. The remainder of the synthesis will proceed as described in the preceding Examples.

Of course, the on-bead cross-metathesis strategy described in PCT Publ. No. WO 2009/015384 to Miller et al., which is hereby incorporated by reference in its entirety, will also permit synthesis of non-symmetrical pseudopeptides (for example, compounds incorporating one amide-linked Phe and one pseudopeptide Phe).

These reduced amide analogs will be prepared, purified, and screened for their binding affinities for HIV-1 FSS. Dimers with high affinity will be tested for their efficacy inhibiting the infectivity and replicative ability of pseudotyped HIV as described in Example 9 above.

### Example 13

### Ternary Resin-Bound Dynamic Combinatorial Chemistry

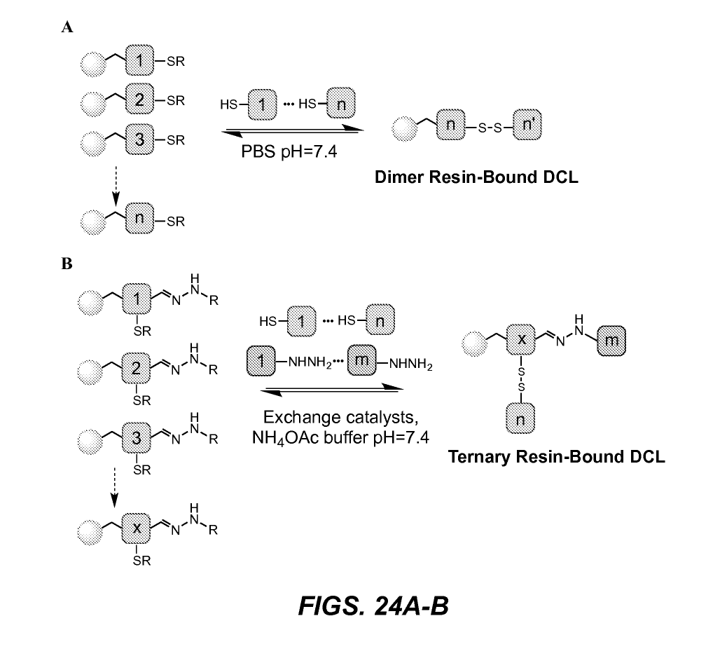

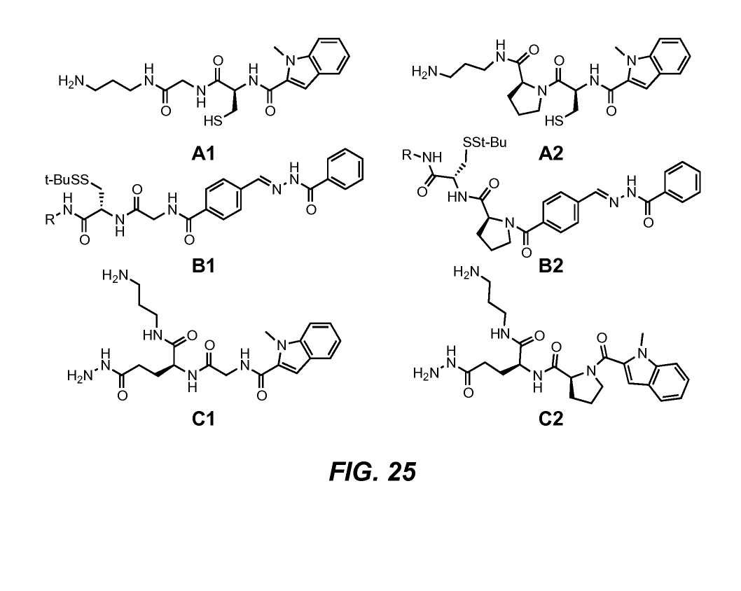

It would be advantageous to extend the RBDCC technique to larger, nondimeric libraries. In many of the earliest DCL experiments, library monomers were synthesized carrying both partners of a chemically reactive pair (i.e., alcohol/ester, thiol/thiol, amine/aldehyde, etc.) allowing stoichiometric production of higher-order oligomers. In the context of expanding the range of RBDCC, this would render identification of selected compounds difficult. An alternative and increasingly popular strategy involves the use of two orthogonally addressable exchange chemistries. In principle, this reduces the potential for entropic bias favoring dimers over trimers, since one can run the DCL selection as two successive processes, one for each type of exchange chemistry, as well as in a simultaneous exchange mode. The success of aniline catalysis as a method for accelerating acyl hydrazone exchange is particularly notable, as this potentially facilitates the use of simultaneous hydrazone and disulfide exchange at biopolymer-friendly pH values. To test the viability of this concept in a resin-bound format, the generic nucleic acid-targeted library design as shown in FIG. 24B was modified. An initial set of monomers was designed (FIG. 25) containing a thiol (A1, A2), an acyl hydrazine (C1, C2), or both a thiol and acyl hydrazone (B1, B2). It was expected that use of glycine and proline as the two “variable” amino acids would provide sufficient structural differences to allow separation of library components by HPLC, without introducing the reactivity and protecting group complications that might occur through the use of amino acids with heteroatom-containing side chains. Monofunctional monomers A1, A2, C1, and C2 incorporated an aromatic moiety (here N-methyl indole carboxylic acid) to provide a readily observable chromophore for HPLC analysis and by analogy to the heterocyclic groups included in previous RNA- and DNA-targeted libraries. Library components were synthesized on Wang resin using standard solid-phase Fmoc peptide synthesis protocols. Following cleavage, the structures of solution-phase monomers were confirmed by mass spectrometry.

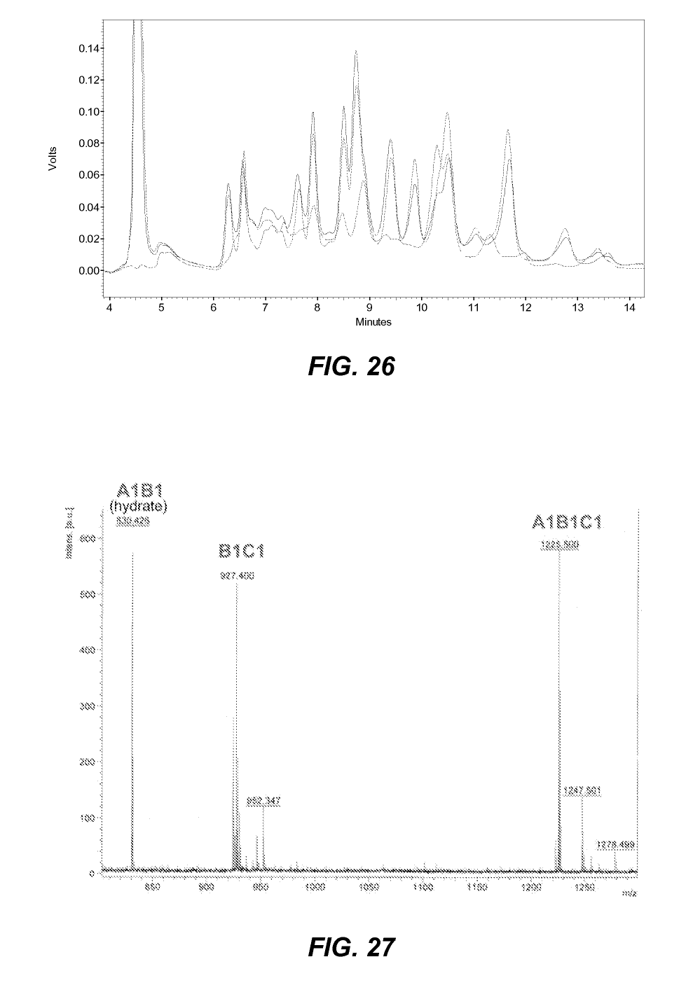

Before attempting experiments with immobilized components, the behavior of the library was studied in a fully solution-phase system. Both thiopropanol and glutathione were tested as facilitators of disulfide exchange, while aniline was employed as a catalyst for hydrazone exchange. Equilibration of A1 and A2 with B1 and B2 (0.13 mM each, 1:1:1:1 ratio) in the presence of one equivalent of thiopropanol in 50 mM ammonium acetate buffer (pH 7.4), containing 5.5% DMSO, for 3 days yielded several new product peaks in the HPLC trace (FIG. 26, green trace). MALDI data confirmed formation of several desired disulfides AB as well as AA, BB, and thiopropanol adducts. Similarly, reaction of B1 and B2 with C1 and C2 (0.13 mM each, 1:1:1:1 ratio) in a pH 7.4 solution yielded the exchanged hydrazones in the presence of 37.5 equivalents of 35 aniline (FIG. 26, red trace). This reaction occurred much slower in the absence of aniline, consistent with the expected lack of reactivity of the uncatalyzed system at this pH.

Simultaneous disulfide and acyl hydrazone exchange was examined by mixing monomers A1 (thiol exchange), C1 (acyl hydrazone exchange), and B1 (bidirectional exchange) with thiopropanol and aniline and allowing them to equilibrate for 3 days. Formation of dimer B1C1 and trimer A1B1C1 were observed by MALDI. This complexity was increased further through the inclusion of all four monofunctional monomers (A1, A2, C1, C2), the two bifunctional monomers B1 and B2, and exchange catalysts thiopropanol and aniline. Following equilibration, two different peaks corresponding to mixed ABC structures were observed by MALDI-MS. A1B1C1 and A2B2C2, the ABC trimers expected to be in lowest concentration based on a statistical distribution of products, were not detected. Incorporating a 10-fold higher concentration of A and C monomers (building blocks A1, A2, B1, B2, C1, C2 in a 10:10:1:1:10:10 ratio) provided an overall increase in the formation of exchange products as measured by HPLC, although MALDI-MS did not change significantly.

With a successful demonstration of simultaneous bidirectional disulfide and hydrazone exchange in the model solution library, evaluation of the system was carried out in a solid-phase context. Building blocks B1 and B2 were synthesized on TentaGel Macrobead resin, linked via aminohexanoic acid (R) to a photocleavable linker (2-nitrophenyl glycine) using standard solid-phase Fmoc peptide synthesis protocols. Incorporation of the aminohexanoic acid and photocleavable linker attachment was done in order to structure the test compounds as closely as possible to what would be used in a more diverse library, which would require this sort of attachment to allow for acid-mediated deprotection of amino acid sidechains without cleavage from the resin. After cleaving portions of the resin for each monomer and verifying identity by mass spectrometry, several exchange experiments were set up similar to the solution phase experiments described above.

First, control experiments were carried out in which only either hydrazone or disulfide exchange was allowed to occur. For hydrazone exchange, resin-bound building block B1 was allowed to equilibrate with the solution phase building blocks C1 and C2 in the presence of aniline for 6 days. For disulfide exchange, resin-bound building block B1 was first pretreated with 20 equivalents of thiopropanol for 24 h, then allowed to equilibrate with solution phase building block A1 for 6 days. Following equilibration, solution phase library components were drained from the resin, which was then washed with water (×3), DMF (×3), THF (×3), and CH3CN (×3) and resuspended in a 4:1 mixture of acetonitrile:methanol. Photocleavage using a compact UV lamp was allowed to proceed for 6-8 hours, after which samples were analyzed by HPLC and MALDI-MS. In both cases formation of either AB or BC dimer products was observed.

Next, a simultaneous exchange experiment was attempted using 3 building blocks, one of each kind (Al and Cl in solution, B1 on resin) in a 1:1:1 ratio. However, only dimers A1B1 and B1C1 were observed following as much as 6 days of equilibration. This result can be attributed to slower kinetics for the resin-bound exchange. In order to facilitate the exchange reactions, the concentration of solution phase building blocks was increased 10 fold (10:1:10 ratio). In this case, after 6 days of equilibration formation of the desired trimer A1B1C1 was observed (FIG. 27).

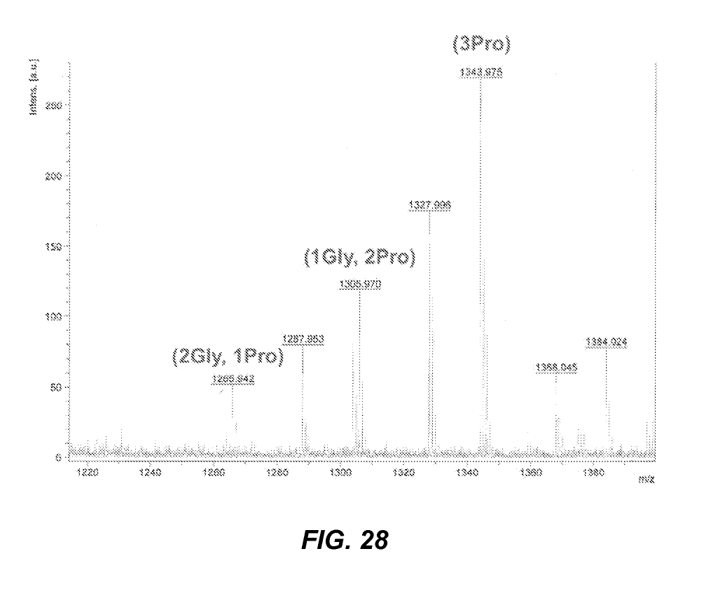

Finally, a full library equilibration experiment was set up in quadruplicate (2 experiments for 1 week of equilibration, and 2 experiments for 2 weeks). Two resin-bound, thiopropanol modified, bi-functional building blocks B1 and B2 were allowed to equilibrate for 1 or 2 weeks with solution phase thiol building blocks A1 and A2 and hydrazone building blocks C1 and C2 (1:1:10:10:10:10) in ammonium acetate buffer (pH=7.4) in the presence of aniline. After equilibration, resin beads were washed, and products were cleaved from the resin. After 1 week of equilibration, 3 different ABC products were detected as well as several dimers AB and BC (FIG. 28). Trimer A1B1C1 (M+H=1225.5, all 3 varied amino acids were glycine), could not be detected. Comparison of HPLC traces along with MALDI data for duplicate experiments equilibrated for 1 week with those equilibrated for 2 weeks showed that additional reaction time does not significantly alter library composition.

Successful proof-of-concept experiments for orthogonal exchange chemistries operating individually and simultaneously have been shown across a three-component phase-segregated dynamic combinatorial library, or “ternary RBDCC”. Use of resin-bound monomers was found to require increases in the concentration of solution monomers in order to observe the formation of trimers. As library equilibration was conducted near neutral pH in buffered solution, it is expected that ternary RBDCC will prove useful for screening libraries intended to yield novel protein and nucleic acid binding molecules.

Although the invention has been described in detail for the purposes of illustration, it is understood that such detail is solely for that purpose, and variations can be made therein by those skilled in the art without departing from the spirit and scope of the invention, which is defined by the following claims.

1. A homo- or hetero-dimer compound comprising a disulfide bond, sulfinyl thio linkage, olefin bond, or hydrocarbon bond linking two monomers having a structure 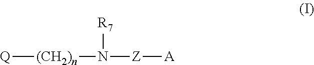
wherein, for each monomer (I)
Q is independently selected from H, NH₂, and 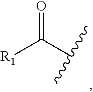
wherein R₁ is selected from a straight or branched chain C₁ to C₆ hydrocarbon and an aromatic or heteroaromatic group;
n is independently an integer from 0 to about 5;
R₇ is selected from hydrogen and an aromatic or heteroaromatic group;
Z is a peptide containing at least two and up to about ten amino acids, where one of the amino acids is linked to an amino acid in the other monomer by a disulfide bond, sulfinyl thio linkage, olefin bond, or hydrocarbon bond; and
A is independently hydrogen or an aromatic or heteroaromatic group connected to Z via a carbonyl linkage, provided that in at least one of the monomers A is not hydrogen and is selected from a substituted or unsubstituted benzo[g]quinoline, benzo[b][1,8]naphthyridine, and anthyridine.

2. The dimer compound according to claim 1, wherein when A is not a substituted or unsubstituted benzo[g]quinoline, benzo[b][1,8]naphthyridine, or anthyridine, A is selected from hydrogen, 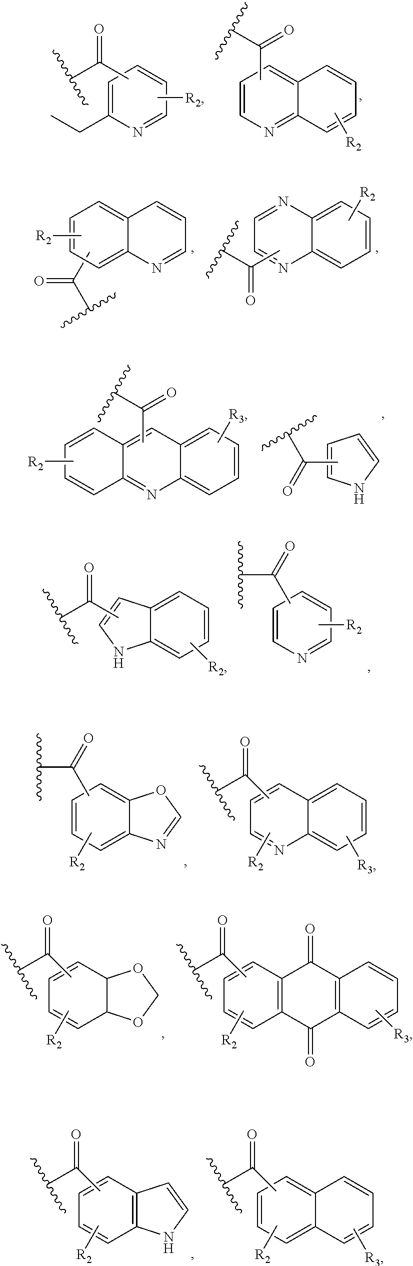 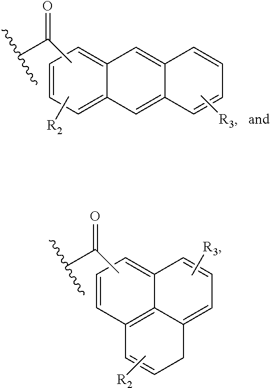
wherein
R₂ and R₃ are independently selected from the group consisting of hydrogen, methyl, ethyl, propyl, amino, methylamine, ethylamine, dimethylamine, diethylamine, methoxy, ethoxy, propoxy, hydroxyl, cyano, and thiocyanato.

3. The dimer compound according to claim 1, wherein Z is a dipeptide, a tripeptide, or a tetrapeptide.

4. The dimer compound according to claim 3, wherein peptides of the dipeptide, tripeptide, or tetrapeptide are independently selected from a peptide, N-methylated peptide, or reduced peptide.

5-6. (canceled)

7. The dimer compound according to claim 1, wherein the amino acids are selected from Cys, His, Lys, Phe, Ala, Ser, Asp, Asn, Val, Pro, Thr, Met, Gly, and D amino acids, N-methyl amino acids, and pseudo amino acids thereof.

8. The dimer compound according to claim 1, wherein the two monomers are linked together by a disulfide bond or sulfinyl thio linkage.

9. The dimer compound according to claim 1, wherein the two monomers are linked together by an olefin bond.

10. (canceled)

11. The dimer compound according to claim 1, wherein the dimer compound is a homo- or hetero-dimer comprising an olefin bond linking together two monomers having a structure 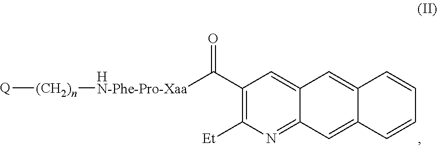
wherein, for each monomer (II)
Xaa is an α-amino acid comprising an unsaturated hydrocarbon sidechain;
Q is independently selected from H and NH₂; and
n is independently an integer from 0 to about 5.

12. The dimer compound according to claim 1, wherein the dimer compound is a homo- or hetero-dimer comprising an olefin bond linking together two monomers having a structure 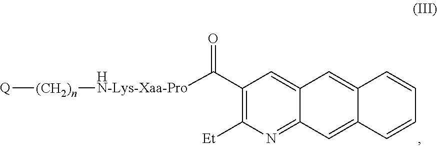
wherein, for each monomer (III)
Xaa is an α-amino acid comprising an unsaturated hydrocarbon sidechain;
Q is independently selected from H and NH₂; and
n is independently an integer from 0 to about 5.

13. The dimer compound according to claim 1, wherein the dimer compound is a homo- or hetero-dimer comprising a disulfide bond linking together two monomers having a structure 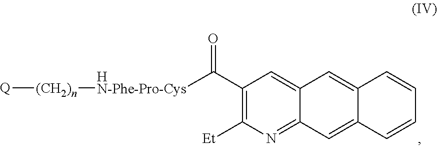
wherein, for each monomer (IV)
Q is independently selected from H and NH₂ and
n is independently an integer from 0 to about 5.

14. A method of inhibiting HIV-1 proliferation, said method comprising:
providing a dimer compound according to claim 1 and
contacting an HIV-1 mRNA that encodes Pol polyprotein with the dimer compound under conditions effective to alter normal expression of the Pol polyprotein and thereby inhibit HIV-1 proliferation.

15. The method according to claim 14, wherein the dimer compound selectively binds an HIV-1 frameshift regulatory sequence.

16. The method according to claim 14, wherein said contacting is carried out in vitro.

17. The method according to claim 14, wherein said contacting is carried out in vivo.

18. A method of treating HIV-1 in a human patient, said method comprising:
administering to a human patient a dimer compound according to claim 1 under conditions effective to alter normal expression of HIV-1 Pol polyprotein, thereby disrupting HIV-1 proliferation to treat the human patient for HIV-1.

19. The method according to claim 18, wherein the dimer compound selectively binds an HIV-1 mRNA frameshift regulatory sequence.

20-42. (canceled)

43. A composition comprising a homo- or hetero-dimer compound according to claim 1 and a carrier.

44. The composition according to claim 43, wherein the carrier is a pharmaceutically-acceptable carrier.

45. The composition according to claim 43, wherein the carrier is selected from a polyethylene glycol conjugate, a liposome, oligoarginine, and a nanoparticle carrier.

46-73. (canceled)


SEQUENCE LISTING (9 sequences)

SEQ ID NO: 1 - RNA, 13 nt, artificial
  (CUG)2 DM1 RNA Repeat
cccgcugcugcgg

SEQ ID NO: 2 - RNA, 19 nt, artificial
  (CUG)4 DM1 RNA Repeat
cccgcugcugcugcugcgg

SEQ ID NO: 3 - RNA, 37 nt, artificial
  (CUG)10 DM1 RNA Repeat
cccgcugcugcugcugcugcugcugcugcugcugcgg

SEQ ID NO: 4 - RNA, 47 nt, artificial
  (CUG)10 DM2 RNA Repeat
cccgccugccugccugccugccugccugccugccugccugccugcgg

SEQ ID NO: 5 - DNA, 37 bp, artificial
  (CAG)10 Control Sequence
cccgcagcagcagcagcagcagcagcagcagcagcgg

SEQ ID NO: 6 - RNA, 37 nt, artificial
  CUG-CAG Duplex Control Sequence
cccgcugcugcugcugcugcagcagcagcagcagcgg

SEQ ID NO: 7 - RNA, 22 nt, artificial
  gag/pol frameshift-stimulating region control sequence
ggccuucccacaagggaaggcc

SEQ ID NO: 8 - DNA, 9 bp, artificial
  Noncoding expanded repeat sequence in diabetes mellitus
acaggggts

SEQ ID NO: 9 - DNA, 11 bp, artificial
  Noncoding expanded repeat seqence in myoclonus epilepsy
ccccgccccgc